(19) United States
(12) Patent Application Publication
(10) Pub. No.: US 2010/0105663 A1
(43) Pub. Date: Apr. 29, 2010

(54) 2-(HETEROARYL) ALKYL INDAZOLE 6-PHENYL AND THIENYL METHYL AMIDE AS THROMBIN INHIBITORS

(75) Inventors: Stephan Siegel, Wuppertal (DE); Dirk Schneider, Wuppertal (DE); Anja Buchmüller, Essen (DE); Elke Dittrich-Wengenroth, Wuppertal (DE); Chrisstoph Gerdes, Leverkusen (DE); Mark Jean Gnoth, Mettmann (DE); Stefan Heitmeier, Wülfrath (DE); Martin Hendrix, Berkeley (DE); Ulrich Rester, Wuppertal (DE); Uwe Saatmann, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, LEVERKUSEN (DE)

(21) Appl. No.: 12/373,677

(22) PCT Filed: Jul. 2, 2007
(86) PCT No.: PCT/EP07/05832

§ 371 Date: Dec. 2, 2009

(30) Foreign Application Priority Data
Jul. 14, 2006 (DE) 10 2006 032 824.8

(51) Int. Cl. A61K 31/55; C07D 231/56; A61K 31/416; C07D 277/20; A61K 31/426; C07D 401/02; A61K 31/4439; C07D 263/04; A61K 31/421; C07D 249/08; A61K 31/4196; C07D 413/02; A61K 31/5377; C07D 401/14; A61K 31/444; C07D 403/02; A61K 31/496; A61P 9/00

(52) U.S. Cl. 514/217.09; 548/361.1; 514/406; 548/204; 514/365; 546/275.7; 514/338; 548/229; 514/376; 548/266.4; 514/383; 544/140; 514/234.5; 546/256; 514/333; 544/371; 514/254.06; 540/603; 514/236.8

(57) ABSTRACT

The invention relates to substituted indazoles and methods for production thereof and use thereof for the production of medicinal products for the treatment and/or prophylaxis of diseases, especially of cardiovascular diseases, preferably of thromboembolic diseases.

The invention relates to substituted indazoles and methods of their production and their use for the production of medicinal products for the treatment and/or prophylaxis of diseases, in particular of cardiovascular diseases, preferably of thromboembolic diseases.

Coagulation (haemostasis) is a defense mechanism of the body, with the aid of which defects in the vessel wall can be “sealed up” quickly and reliably. In this way, in the intact organism, blood loss and organ damage are avoided or minimized after injury. After a vessel is injured, haemostasis takes place on the one hand through activation of thrombocytes, and on the other hand by means of the coagulation system, in which an enzymatic cascade of complex reactions of plasma proteins is initiated. Numerous coagulation factors take part in this, and each of them, once activated, transforms the respective next inactive precursor into its active form. In this series of reactions the activated serine protease factor Xa (FXa) or the FXa-containing prothrombinase complex finally cleaves prothrombin to thrombin, which in its turn cleaves the soluble fibrinogen and transforms it into the insoluble form of fibrin and so forms the actual blood clot.

Furthermore, through the proteolytic activation of platelet receptors, thrombin is a potent trigger of thrombocyte aggregation, which also makes an important contribution to haemostasis. Other functions of thrombin, which contribute to coagulation, are stabilization of the fibrin clot through activation of factor XIII, intensification of the coagulation reaction by activation of cofactors V and VIII, and inhibition of fibrinolysis through activation of procarboxypeptidase B (syn. TAFI). Finally, through proteolytic activation of protein C, thrombin can counteract excessive activity of the coagulation cascade and therefore excessive haemostasis (thrombosis).

In the course of many cardiovascular and metabolic diseases, however, because of systemic factors, e.g. hyperlipidaemia, diabetes or smoking, as a result of blood flow changes with stasis, e.g. in atrial fibrillation, or as a result of pathological changes in the vessel wall, e.g. endothelial dysfunctions or atherosclerosis, there is an increased tendency to coagulation and thrombocyte activation. This undesirable and excessive haemostasis can, through formation of thrombi rich in fibrin and platelets, lead to thromboembolic diseases and thrombotic complications with life-threatening states.

The anticoagulants, i.e. substances for inhibiting or preventing coagulation, that are known from the prior art have various, often serious disadvantages. An efficient method of treatment or prophylaxis of thromboembolic diseases therefore proves in practice to be very difficult and unsatisfactory (D. A. Lane, et al., Directing Thrombin. Blood 106, 2605-2612, 2005; D. Gustafsson, et al., Nature Reviews Drug Discovery, 3, 649-659, 2004; L. Wallentin, et al., The Lancet 362, 789-797, 2003).

For the therapy and prophylaxis of thromboembolic diseases, on the one hand heparins are used, which are administered parenterally or subcutaneously. Owing to more favourable pharmacokinetic properties, low-molecular heparin is now increasingly preferred, but even so, the known disadvantages described below, which occur during treatment with heparin, cannot be avoided. Thus, heparin is not effective orally, and only has a comparatively short half-life. As heparin inhibits several factors of the coagulation cascade simultaneously, the action is nonselective. Furthermore, there is a risk of haemorrhage, and in particular there may be cerebral haemorrhages and haemorrhages in the gastrointestinal tract, and there may be thrombocytopenia, alopecia medicamentosa or osteoporosis.

The vitamin K-antagonists represent a second class of anticoagulants. These include, for example, 1,3-indanediones, but mainly compounds such as warfarin, phenprocoumon, dicumarol and other coumarin derivatives, which nonselectively inhibit the synthesis of various products of certain vitamin K-dependent coagulation factors in the liver. Owing to the mechanism of action, the effects only develop very slowly (latent period to onset of action 36 to 48 hours). The compounds can indeed be administered orally, but because of the high risk of haemorrhage and the narrow therapeutic index, expensive individual adjustment and observation of the patient are required. Furthermore, other side effects such as gastrointestinal disturbances, hair loss and skin necroses have been described.

Newer approaches for oral anticoagulants are in various phases of clinical testing or in clinical use, but they have also shown disadvantages, e.g. highly variable bioavailability, liver damage and haemorrhagic complications.

EP-A 0 574 174 describes, among others, indazoles as angiotensin II antagonists for the treatment of hypertension.

One object of the present invention is therefore to provide novel compounds as thrombin inhibitors for the treatment of cardiovascular diseases, in particular thromboembolic diseases, in humans and animals, which have a large therapeutic spectrum.

The invention relates to compounds of formula

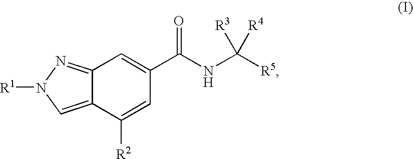

in which
R¹ stands for a compound of formula

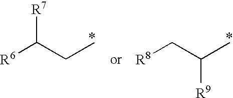

where * is the site of linkage to the indazole,
R⁶ stands for C₁-C₆-alkyl, C₃-C₈-cycloalkyl, phenyl, 5- to 7-membered heterocyclyl or 5- or 6-membered heteroaryl,
   in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxy-carbonyl and C₁-C₄-alkylaminocarbonyl,
R⁷ stands for hydrogen, C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
   in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
      in which cycloalkyl and heterocyclyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
      and
      in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which heterocyclyl and heterocyclylcarbonyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
   and
   in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxy-carbonyl,
R⁸ stands for C₁-C₆-alkyl, C₃-C₈-cycloalkyl, phenyl, 5- to 7-membered heterocyclyl or 5- or 6-membered heteroaryl,
   in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxy-carbonyl and C₁-C₄-alkylaminocarbonyl,
R⁹ stands for C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
   in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
      in which cycloalkyl and heterocyclyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
      and
      in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
and
in which heterocyclyl and heterocyclylcarbonyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
and
in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
R² stands for hydrogen, halogen, cyano, trifluoromethyl, trifluoromethoxy, trifluoromethylthio, C₁-C₃-alkyl, C₁-C₃-alkoxy, C₁-C₃-alkylthio or cyclopropyl,
   in which alkyl, alkoxy, alkylthio and cyclopropyl can be substituted with 1 to 3 substituents, the substituents being selected independently of one another from the group comprising halogen,
R³ stands for hydrogen or C₁-C₄-alkyl,
R⁴ stands for hydrogen or C₁-C₄-alkyl,
or
R³ and R⁴ form, together with the carbon atom to which they are bound, a cyclopropyl ring or a cyclobutyl ring,
R⁵ stands for phenyl, 2-thienyl or 3-thienyl,
   in which phenyl can be substituted with 1 to 3 substituents, the substituents being selected independently of one another from the group comprising halogen, methyl, ethinyl, methoxy and 1,2,4-triazol-1-yl,
   in which methoxy can be substituted with a substituent, the substituent being selected from the group comprising C₁-C₄-alkoxycarbonyl, C₁-C₄-alkylaminocarbonyl and C₃-C₆-cycloalkylaminocarbonyl,
   and

in which 2-thienyl and 3-thienyl can be substituted with 1 to 3 substituents, the substituents being selected independently of one another from the group comprising halogen, methyl, ethinyl and methoxy,

and their salts, their solvates and the solvates of their salts.

Compounds according to the invention are the compounds of formula (I) and their salts, solvates and solvates of the salts, and the compounds covered by formula (I), called example(s) of application below, and their salts, solvates and solvates of the salts, provided the compounds stated below, covered by formula (I), are not already salts, solvates and solvates of the salts.

The compounds according to the invention can, depending on their structure, exist in stereoisomeric forms (enantiomers, diastereomers). The invention therefore includes the enantiomers or diastereomers and mixtures thereof The stereoisomerically uniform constituents can be isolated in a known manner from such mixtures of enantiomers and/or diastereomers.

If the compounds according to the invention can occur in tautomeric forms, the present invention includes all tautomeric forms.

Physiologically harmless salts of the compounds according to the invention are preferred as salts within the scope of the present invention. However, salts which themselves are not suitable for pharmaceutical uses but for example can be used for the isolation or purification of the compounds according to the invention, are also included.

Physiologically harmless salts of the compounds according to the invention comprise salts of acid addition of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoracetic acid, propionic acid, lactic acid, tartaric acid, malic acid, citric acid, fumaric acid, malic acid and benzoic acid.

Physiologically harmless salts of the compounds according to the invention also include salts of the usual bases, for example and preferably alkali metal salts (e.g. sodium and potassium salts), alkaline-earth salts (e.g. calcium and magnesium salts) and ammonium salts, derived from ammonia or organic amines with 1 to 16 carbon atoms, for example and preferably ethylamine, diethylamine, triethylamine, ethyldiisopropyl-amine, monoethanolamine, diethanolamine, triethanolamine, dicyclohexylamine, dimethylaminoethanol, procaine, dibenzylamine, N-methylmorpholine, arginine, lysine, ethylene diamine, N-methylpiperidine and choline.

Solvates are, within the scope of the invention, those forms of the compounds according to the invention that form a complex in the solid or liquid state by coordination with solvent molecules. Hydrates are a special form of solvates, in which the coordination takes place with water.

In addition, the present invention also includes prodrugs of the compounds according to the invention. The term “prodrugs” comprises compounds which can themselves be biologically active or inactive, but during their residence time in the body they are converted (for example metabolically or hydrolytically) to compounds according to the invention.

Within the scope of the present invention, the substituents, unless specified otherwise, have the following meaning:

Alkyl per se and “alk” and “alkyl” in alkoxy, alkylamino, alkylthio, alkylcarbonyl, alkoxycarbonyl, alkylaminocarbonyl and alkylcarbonylamino stand for a linear or branched alkyl residue with 1 to 6, preferably with 1 to 4 carbon atoms, for example and preferably for methyl, ethyl, n-propyl, iso-propyl, n-butyl, tert-butyl, n-pentyl and n-hexyl.

Alkoxy stands, for example and preferably, for methoxy, ethoxy, n-propoxy, iso-propoxy, n-butoxy and tert-butoxy.

Alkylamino stands for an alkylamino residue with one or two (selected independently of one another) alkyl substituents, for example and preferably for methylamino, ethylamino, n-propylamino, iso-propylamino, tert-butylamino, n-pentylamino, n-hexylamino, N,N-dimethylamino, N,N-diethylamino, N-ethyl-N-methylamino, N-methyl-N-n-propylamino, N-iso-propyl-N-n-propylamino, N-tert-butyl-N-methylamino, N-ethyl-N-n-pentylamino and N-n-hexyl-N-methyl-amino. C₁-C₃-alkylamino stands for example for a monoalkylamino residue with 1 to 3 carbon atoms or for a dialkylamino residue with 1 to 3 carbon atoms per alkyl substituent.

Alkylthio stands for example and preferably for methylthio, ethylthio, n-propylthio, isopropylthio, tert.-butylthio, n-pentylthio and n-hexylthio.

Alkylcarbonyl stands for example and preferably for methylcarbonyl, ethylcarbonyl, n-propylcarbonyl, iso-propylcarbonyl, n-butylcarbonyl and tert-butylcarbonyl.

Alkoxycarbonyl stands for example and preferably for methoxycarbonyl, ethoxycarbonyl, n-propoxycarbonyl, iso-propoxycarbonyl, n-butoxycarbonyl, tert-butoxycarbonyl, n-pentoxycarbonyl and n-hexoxycarbonyl.

Alkylaminocarbonyl stands for an alkylaminocarbonyl residue with one or two (selected independently of one another) alkyl substituents, for example and preferably for methylaminocarbonyl, ethylaminocarbonyl, n-propylaminocarbonyl, iso-propylaminocarbonyl, tert-butylaminocarbonyl, n-pentylaminocarbonyl, n-hexylaminocarbonyl, N,N-dimethylaminocarbonyl, N,N-diethylamino-carbonyl, N-ethyl-N-methylaminocarbonyl, N-methyl-N-n-propylaminocarbonyl, N-iso-propyl-N-n-propylaminocarbonyl, N-tert-butyl-N-methylaminocarbonyl, N-ethyl-N-n-pentylaminocarbonyl and N-n-hexyl-N-methyl-aminocarbonyl. C₁-C₃-alkylaminocarbonyl stands for example for a monoalkylaminocarbonyl residue with 1 to 3 carbon atoms or for a dialkylaminocarbonyl residue with 1 to 3 carbon atoms per alkyl substituent.

Alkylcarbonylamino stands for example and preferably for methylcarbonylamino, ethylcarbonylamino, n-propylcarbonylamino, iso-propylcarbonylamino, n-butylcarbonylamino and tert-butylcarbonylamino.

Cycloalkyl stands for a mono- or bicyclic cycloalkyl group with as a rule 3 to 8, preferably 3, 5 or 6 carbon atoms, for example and preferably for cycloalkyl we may mention cyclopropyl, cyclobutyl, cyclopentyl, cyclohexyl and cycloheptyl.

Cycloalkylaminocarbonyl stands for example and preferably for cyclopropylaminocarbonyl, cyclobutylaminocarbonyl, cyclopentylaminocarbonyl and cyclohexylaminocarbonyl.

Heterocyclyl stands for a monocyclic, heterocyclic residue with as a rule 5 to 7 ring atoms and up to 3, preferably up to 2 heteroatoms and/or hetero groups from the series N, O, S, SO, SO₂, where a nitrogen atom can also form an N-oxide. The heterocyclyl residues can be saturated or partially unsaturated. 5- to 7-membered, monocyclic saturated heterocyclyl residues are preferred with up to two heteroatoms from the series O, N and S, for example and preferably for pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, per-hydroazepinyl, piperazin-1-yl, piperazin-2-yl.

Heteroaryl stands for an aromatic, monocyclic residue with as a rule 5 or 6 ring atoms and up to 4 heteroatoms from the series S, O and N, where a nitrogen atom can also form an N-oxide, for example and preferably for thienyl, furyl, pyrrolyl, thiazolyl, oxazolyl, oxadiazolyl, pyrazolyl, imidazolyl, pyridyl, pyrimidyl, pyridazinyl, pyrazinyl.

Halogen stands for fluorine, chlorine, bromine and iodine, preferably for fluorine and chlorine.

In the formulae of the group that can stand for R¹, the end point of the line next to which there is a *, does not stand for a carbon atom or a CH₂ group, but is a component of the bond to the atom to which R¹ is bound.

Compounds of formula (I) are preferred in which
R¹ stands for a compound of formula

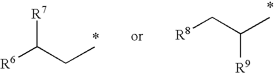

where * is the site of linkage to the indazole,
R⁶ stands for C₁-C₆-alkyl, C₃-C₈-cycloalkyl, phenyl, 5- to 7-membered heterocyclyl or 5- or 6-membered heteroaryl,
   in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxy-carbonyl and C₁-C₄-alkylaminocarbonyl,
R⁷ stands for hydrogen, C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
   in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
      in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
      and
   in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which heterocyclyl and heterocyclylcarbonyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
R⁸ stands for C₁-C₆-alkyl, C₃-C₈-cycloalkyl, phenyl, 5- to 7-membered heterocyclyl or 5- or 6-membered heteroaryl,
   in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxy-carbonyl and C₁-C₄-alkylaminocarbonyl,
R⁹ stands for C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
   in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
      in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
      and
   in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
   and
   in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
R² stands for hydrogen, halogen, cyano, trifluoromethyl, trifluoromethoxy, trifluoromethylthio, C₁-C₃-alkyl, C₁-C₃-alkoxy, C₁-C₃-alkylthio or cyclopropyl,
   in which alkyl, alkoxy, alkylthio and cyclopropyl can be substituted with 1 to 3 substituents, the substituents being selected independently of one another from the group comprising halogen,
R³ stands for hydrogen or C₁-C₄-alkyl,
R⁴ stands for hydrogen or C₁-C₄-alkyl,
or
R³ and R⁴ form, together with the carbon atom to which they are bound, a cyclopropyl ring or a cyclobutyl ring,
R⁵ stands for phenyl, 2-thienyl or 3-thienyl,
   in which phenyl, 2-thienyl and 3-thienyl can be substituted with 1 to 3 substituents, the substituents being selected independently of one another from the group comprising halogen, methyl, ethinyl and methoxy,
and their salts, their solvates and the solvates of their salts.

Compounds of formula (I) are also preferred in which
R¹ stands for a compound of formula

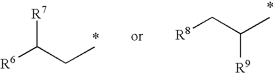

where * is the site of linkage to the indazole,
R⁶ stands for C₁-C₆-alkyl, phenyl, 5- to 7-membered heterocyclyl or 5- or 6-membered heteroaryl,
   in which heterocyclyl can be substituted with 1 to 3 substituents, the substituents being selected independently of one another from the group comprising halogen, oxo, C₁-C₄-alkyl, C₁-C₄-alkoxy and C₁-C₄-alkylamino,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylamino and C₁-C₄-alkylaminocarbonyl,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with 1 to 3 substituents, the substituents being selected independently of one another from the group comprising halogen, oxo, C₁-C₄-alkyl, C₁-C₄-alkoxy, C₁-C₄-alkylamino, C₁-C₄-alkylcarbonyl and C₁-C₄-alkoxycarbonyl,
      and
      in which phenyl and heteroaryl can be substituted with 1 to 3 substituents, the substituents being selected independently of one another from the group comprising halogen, cyano, trifluoromethyl, trifluoromethoxy, C₁-C₄-alkyl, C₁-C₄-alkoxy, C₁-C₄-alkylamino, C₁-C₄-alkylcarbonyl and C₁-C₄-alkoxycarbonyl,
   and
   in which heterocyclyl and heterocyclylcarbonyl can be substituted with 1 to 3 substituents, the substituents being selected independently of one another from the group comprising halogen, oxo, C₁-C₄-alkyl, C₁-C₄-alkoxy, C₁-C₄-alkylamino, C₁-C₄-alkylcarbonyl and C₁-C₄-alkoxycarbonyl,
R⁸ stands for C₁-C₆-alkyl, phenyl, 5- to 7-membered heterocyclyl or 5- or 6-membered heteroaryl,
   in which heterocyclyl can be substituted with 1 to 3 substituents, the substituents being selected independently of one another from the group comprising halogen, oxo, C₁-C₄-alkyl, C₁-C₄-alkoxy and C₁-C₄-alkylamino,
   and
   in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylamino and C₁-C₄-alkylaminocarbonyl,
R⁹ stands for C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with 1 to 3 substituents, the substituents being selected independently of one another from the group comprising halogen, oxo, C₁-C₄-alkyl, C₁-C₄-alkoxy, C₁-C₄-alkylamino, C₁-C₄-alkylcarbonyl and C₁-C₄-alkoxycarbonyl,
      and
   in which phenyl and heteroaryl can be substituted with 1 to 3 substituents, the substituents being selected independently of one another from the group comprising halogen, cyano, trifluoromethyl, trifluoromethoxy, C₁-C₄-alkyl, C₁-C₄-alkoxy, C₁-C₄-alkylamino, C₁-C₄-alkylcarbonyl and C₁-C₄-alkoxycarbonyl,
   and
   in which heterocyclyl and heterocyclylcarbonyl can be substituted with 1 to 3 substituents, the substituents being selected independently of one another from the group comprising halogen, oxo, C₁-C₄-alkyl, C₁-C₄-alkoxy, C₁-C₄-alkylamino, C₁-C₄-alkylcarbonyl and C₁-C₄-alkoxycarbonyl,
R² stands for hydrogen, halogen, cyano, trifluoromethyl, trifluoromethoxy, trifluoromethylthio, C₁-C₃-alkyl, C₁-C₃-alkoxy, C₁-C₃-alkylthio or cyclopropyl,
R³ stands for hydrogen or methyl,
R⁴ stands for hydrogen or methyl,
or
R³ and R⁴ form, together with the carbon atom to which they are bound, a cyclopropyl ring,
R⁵ stands for phenyl, 2-thienyl or 3-thienyl,
   in which phenyl, 2-thienyl and 3-thienyl can be substituted with 1 to 3 substituents, the substituents being selected independently of one another from the group comprising halogen, methyl, ethinyl and methoxy,
and their salts, their solvates and the solvates of their salts.

Compounds of formula (I) are also preferred in which
R¹ stands for a compound of formula

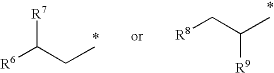

where * is the site of linkage to the indazole,
R⁶ stands for C₁-C₆-alkyl, phenyl, 5- to 7-membered heterocyclyl or 5- or 6-membered heteroaryl,
   in which heterocyclyl can be substituted with 1 to 3 substituents, the substituents being selected independently of one another from the group comprising oxo and C₁-C₄-alkyl,
   and
   in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with 1 to 3 substituents, the substituents being selected independently of one another from the group comprising oxo and C₁-C₄-alkyl,
      and
   in which phenyl and heteroaryl can be substituted with 1 to 3 substituents, the substituents being selected independently of one another from the group comprising halogen, C₁-C₄-alkyl and C₁-C₄-alkoxy,
   and
   in which heterocyclyl and heterocyclylcarbonyl can be substituted with 1 to 3 substituents, the substituents being selected independently of one another from the group comprising oxo and C₁-C₄-alkyl,
R⁸ stands for C₁-C₆-alkyl, phenyl, 5- to 7-membered heterocyclyl or 5- or 6-membered heteroaryl,
   in which heterocyclyl can be substituted with 1 to 3 substituents, the substituents being selected independently of one another from the group comprising oxo and C₁-C₄-alkyl,
   and
   in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁹ stands for C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with 1 to 3 substituents, the substituents being selected independently of one another from the group comprising oxo and C₁-C₄-alkyl,
      and
      in which phenyl and heteroaryl can be substituted with 1 to 3 substituents, the substituents being selected independently of one another from the group comprising halogen, C₁-C₄-alkyl and C₁-C₄-alkoxy,
   and
   in which heterocyclyl and heterocyclylcarbonyl can be substituted with 1 to 3 substituents, the substituents being selected independently of one another from the group comprising oxo and C₁-C₄-alkyl,
R² stands for hydrogen, chlorine, trifluoromethyl, methyl, ethyl or methoxy,
R³ stands for hydrogen or methyl,
R⁴ stands for hydrogen or methyl,
R⁵ stands for phenyl or 2-thienyl,
   in which phenyl and 2-thienyl are substituted with a substituent, the substituent being selected from the group comprising chlorine, fluorine, methyl, ethinyl and methoxy,
and their salts, their solvates and the solvates of their salts.

Compounds of formula (I) are also preferred in which
R¹ stands for a compound of formula

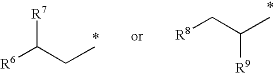

where * is the site of linkage to the indazole,
R⁶ stands for phenyl, 5- to 7-membered heterocyclyl or 5- or 6-membered heteroaryl,
   in which heterocyclyl can be substituted with an oxo substituent,
   and
   in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with an oxo substituent,
      and
      in which phenyl and heteroaryl can be substituted with 1 to 3 substituents, the substituents being selected independently of one another from the group comprising halogen, C₁-C₄-alkyl and C₁-C₄-alkoxy,
   and
   in which heterocyclyl and heterocyclylcarbonyl can be substituted with an oxo substituent,
R⁸ stands for phenyl, 5- to 7-membered heterocyclyl or 5- or 6-membered heteroaryl,
   in which heterocyclyl can be substituted with an oxo substituent,
   and
   in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁹ stands for C₁-C₆-alkyl, C₁-C₄-alkylamino, C₁-C₄-alkoxycarbonyl, 5- to 7-membered heterocyclyl or 5- to 7-membered heterocyclylcarbonyl,
   in which alkyl can be substituted with a substituent, the substituent being selected from the group comprising phenyl, 5- to 7-membered heterocyclyl and 5- to 6-membered heteroaryl,
      in which heterocyclyl can be substituted with an oxo substituent,
      and
      in which phenyl and heteroaryl can be substituted with 1 to 3 substituents, the substituents being selected independently of one another from the group comprising halogen, C₁-C₄-alkyl and C₁-C₄-alkoxy,
   and
   in which heterocyclyl and heterocyclylcarbonyl can be substituted with an oxo substituent,
R² stands for hydrogen or methoxy,
R³ stands for hydrogen,
R⁴ stands for hydrogen,
R⁵ stands for phenyl or 2-thienyl,
   in which phenyl and 2-thienyl are substituted with a substituent, the substituent being selected from the group comprising chlorine, fluorine and methyl,
and their salts, their solvates and the solvates of their salts.

Compounds of formula (I) are also preferred in which R⁷ stands for hydrogen.

Compounds of formula (I) are also preferred in which R² stands for hydrogen, chlorine, trifluoromethyl, methyl, ethyl or methoxy.

Compounds of formula (I) are also preferred in which R² stands for hydrogen, chlorine, methyl or methoxy.

Compounds of formula (I) are also preferred in which R² stands for chlorine, methyl or methoxy.

Compounds of formula (I) are also preferred in which R² stands for hydrogen.

Compounds of formula (I) are also preferred in which R³ stands for hydrogen or methyl.

Compounds of formula (I) are also preferred in which R³ stands for hydrogen.

Compounds of formula (I) are also preferred in which R⁴ stands for hydrogen.

Compounds of formula (I) are also preferred in which R³ and R⁴ stand for hydrogen.

Compounds of formula (I) are also preferred in which R⁵ stands for 3-chlorophenyl.

Compounds of formula (I) are also preferred in which R³ and R⁴ stand for hydrogen and R⁵ stands for 3-chlorophenyl.

The invention further relates to a method of production of the compounds of formula (I), in which according to method

[A] compounds of formula

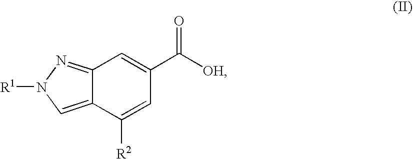

in which

R¹ and R² have the meaning given above,

are reacted with dehydrating reagents with compounds of formula

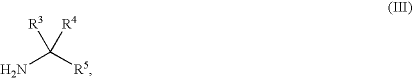

in which

R³, R⁴ and R⁵ have the meaning given above,

or

[B] compounds of formula

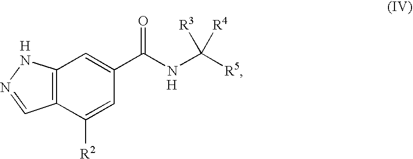

in which

R², R³, R⁴ and R⁵ have the meaning given above,

are reacted with compounds of formula

R¹—X (V),

in which

R¹ has the meaning given above, and
X stands for halogen, preferably bromine or chlorine,

in the presence of a base and then the regioisomers are separated chromatographically,

or

[C] compounds of formula

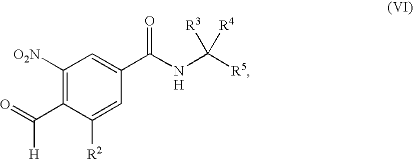

in which

R², R³, R⁴ and R⁵ have the meaning given above,

are reacted with compounds of formula

R¹—NH₂ (VII),

in which

R¹ has the meaning given above,

in a two-stage reaction first with dehydrating reagents with formation of the imine and then are cyclized under reducing conditions.

The reaction according to method [A] is generally carried out in inert solvents, optionally in the presence of a base, preferably in a temperature range from 0° C. to room temperature at normal pressure.

Suitable dehydrating reagents for this are for example carbodiimides e.g. N,N′-diethyl-, N,N′-dipropyl-, N,N′-diisopropyl-, N,N′-dicyclohexylcarbodiimide, N-(3-dimethylaminoisopropyl)-N′-ethylcarbodiimide-hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3-sulphate or 2-tert.-butyl-5-methyl-isoxazolium-perchlorate, or acylamino compounds such as 2-ethoxy-1-ethoxycarbonyl-1,2-dihydroquinoline, or propanephosphonic acid anhydride, or isobutyl-chloroformate, or bis-(2-oxo-3-oxazolidinyl)-phosphoryl chloride or benzotriazolyloxy-tri(dimethyl-amino)phosphonium hexafluorophosphate, or O-(benzotriazol-1-yl)-N,N,N′,N′-tetra-methyluronium-hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylamino-methyliumfluoroborate (TBTU) or O-(7-azabenzotriazol-1-yl)-N,N,N′,N′-tetramethyl-uronium hexafluorophosphate (HATU), or 1-hydroxybenzotriazole (HOBt), or benzotriazol-1-yloxy-Tris(dimethylamino)-phosphonium hexa-fluorophosphate (BOP), or mixtures thereof, with bases. Preferably the condensation is carried out with EDC and HOBt.

Bases are for example alkali carbonates, e.g. sodium or potassium carbonate or hydrogencarbonate, or organic bases such as trialkylamines, e.g. triethylamine, N-methylmorpholine, N-methylpiperidine, 4-dimethylaminopyridine or diisopropylethylamine. Preferably the condensation is carried out with diisopropylethylamine or 4-dimethylaminopyridine.

Inert solvents are for example halohydrocarbons such as dichloromethane or trichloromethane, hydrocarbons such as benzene, or other solvents such as nitromethane, dioxan, dimethylformamide, dimethylsulphoxide or acetonitrile. It is also possible to use mixtures of the solvents. Dichloromethane or dimethylformamide is especially preferred.

The reaction according to method [B] is generally carried out in inert solvents, optionally in the presence of a base, optionally in the presence of potassium iodide, preferably in a temperature range from room temperature up to reflux of the solvents at normal pressure.

Inert solvents are for example halohydrocarbons such as methylene chloride, trichloromethane or 1,2-dichloroethane, ethers such as dioxan, tetrahydrofuran or 1,2-dimethoxyethane, or other solvents such as acetone, dimethylformamide, dimethylacetamide, 2-butanone or acetonitrile, with tetrahydrofuran, methylene chloride, acetone, acetonitrile or dimethylformamide being preferred.

Bases are for example alkali carbonates such as caesium carbonate, sodium or potassium carbonate, or sodium or potassium methanolate, or sodium or potassium ethanolate or potassium tert.-butylate, or amides such as sodium amide, lithium bis-(trimethylsilyl)amide or lithium diisopropylamide, or organometallic compounds such as butyllithium or phenyllithium, or other bases such as sodium hydride, DBU, with potassium tert.-butylate, caesium carbonate, DBU, sodium hydride, potassium carbonate or sodium carbonate being preferred.

The chromatographic separation of the regioisomers is generally carried out by HPLC on a GROM-SIL ODS-4HE, 10 μM stationary phase with a mixture of acetonitrile and water as eluent.

The reaction of the first stage according to method [C] is generally carried out in pure dehydrating reagent without addition of inert solvents, preferably in a temperature range from room temperature to 50° C. at normal pressure.

Dehydrating reagents are for example trimethyl orthoformate or anhydrous alcohols such as ethanol or methanol.

The reaction of the second stage according to method [C] is generally carried out in pure phosphite, phosphonite or phosphorodiamidite, optionally with addition of an inert solvent, preferably in a temperature range from room temperature up to reflux of the solvents at normal pressure.

Phosphites, phosphonites and phosphorodiamidites are for example triethylphosphite, trimethylphosphite, triisopropylphosphite, diethylmethylphosphonite, ethyldiphenylphosphinite or ethyl-N-tetrathylphosphorodiamidite, and triethylphosphite is preferred.

Inert solvents are for example toluene, benzene or xylene.

The compounds of formulae (III), (V) and (VII) are known or can be synthesized by known methods from the corresponding starting compounds.

The compounds of formula (II) are known or can be protected by reacting compounds of formula

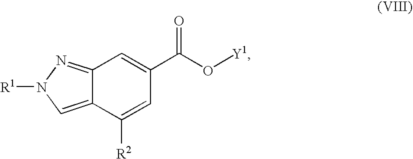

in which

R¹ and R² have the meaning given above, and
Y¹ stands for methyl or ethyl,

with a base.

The reaction is generally carried out in inert solvents, preferably in a temperature range from room temperature up to reflux of the solvents at normal pressure.

Bases are for example alkali hydroxides such as sodium, lithium or potassium hydroxide, or alkali carbonates such as caesium carbonate, sodium or potassium carbonate, and lithium hydroxide is preferred.

Inert solvents are for example halohydrocarbons such as methylene chloride, trichloromethane, tetrachloromethane, trichloroethane, tetrachloroethane, 1,2-dichloroethane or trichloroethylene, ethers such as diethyl ether, methyl-tert.-butyl ether, 1,2-dimethoxyethane, dioxan, tetrahydrofuran, glycol dimethylether or diethylene glycol dimethylether, alcohols such as methanol, ethanol, n-propanol, iso-propanol, n-butanol or tert.-butanol, hydrocarbons such as benzene, xylene, toluene, hexane, cyclohexane or petroleum fractions, or other solvents such as dimethylformamide, dimethylacetamide, dimethylsulphoxide, acetonitrile or pyridine, or mixtures of solvents, methanol or ethanol being preferred.

The compounds of formula (VIII) are known or can be produced by reacting compounds of formula

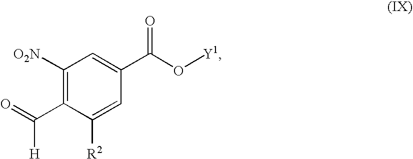

in which

R² and Y¹ have the meaning given above,

with compounds of formula (VII) in a two-stage reaction first with trimethyl orthoformate with formation of the imine and then with triethylphosphite.

The reaction is carried out according to method [C].

The compounds of formula (IX) are known or can be synthesized by known methods from the corresponding starting compounds.

The compounds of formula (IV) are known or can be produced by reacting compounds of formula

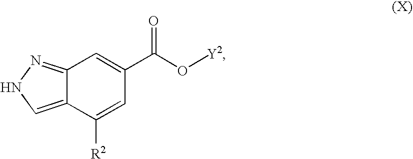

in which
R² has the meaning given above, and
Y² stands for methyl or ethyl,

with compounds of formula (III).

The reaction is generally carried out in inert solvents, in the presence of methylaluminoxane, preferably in a temperature range from room temperature up to reflux of the solvents at normal pressure.

Inert solvents are for example toluene, benzene, xylene or dichloromethane.

The compounds of formula (X) are known or can be synthesized by known methods from the corresponding starting compounds.

The compounds of formula (VI) are known or can be produced by reacting compounds of formula

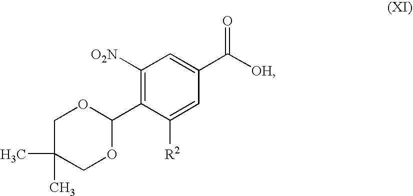

in which

R² has the meaning given above,

with compounds of formula (III) and then the acetal is cleaved with an acid.

The reaction with compounds of formula (III) is carried out according to method [A].

The cleavage of the acetal is generally carried out in the presence an acid, preferably in a temperature range from room temperature to 50° C. at normal pressure.

Acids are for example trifluoracetic acid, hydrochloric acid or sulphuric acid, with a mixture of sulphuric acid and trifluoracetic acid being preferred.

The compounds of formula (XI) are known or can be synthesized by known methods from the corresponding starting compounds.

The production of the starting compounds and of the compounds of formula (I) can be illustrated by the following synthesis schemes.

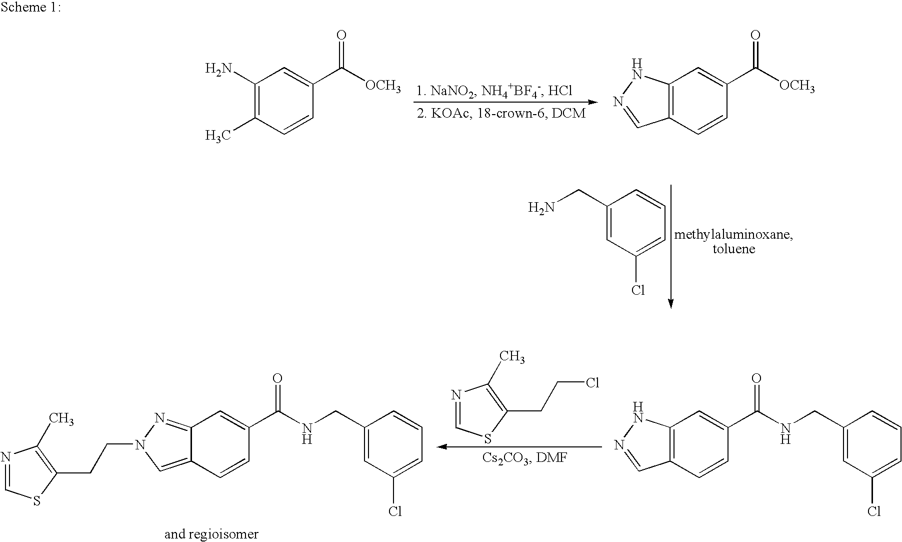

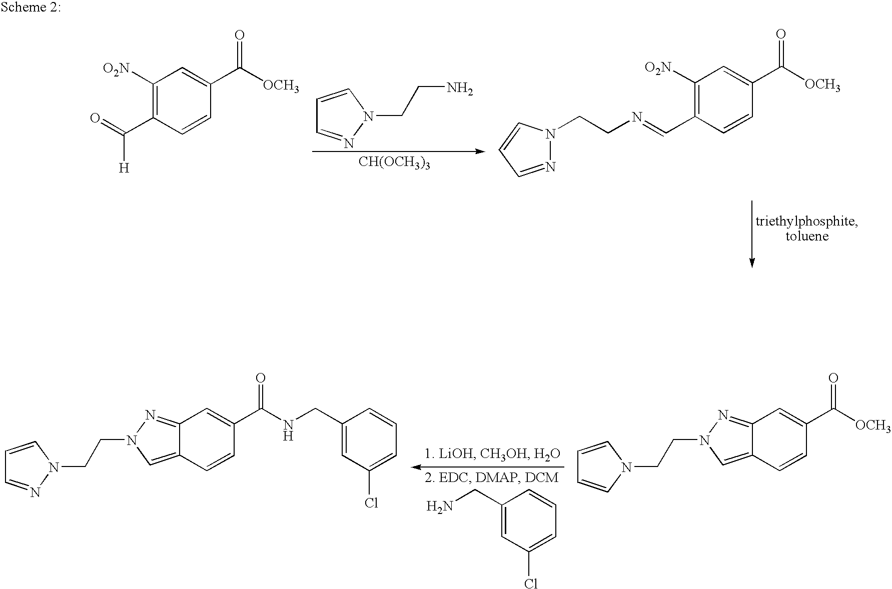

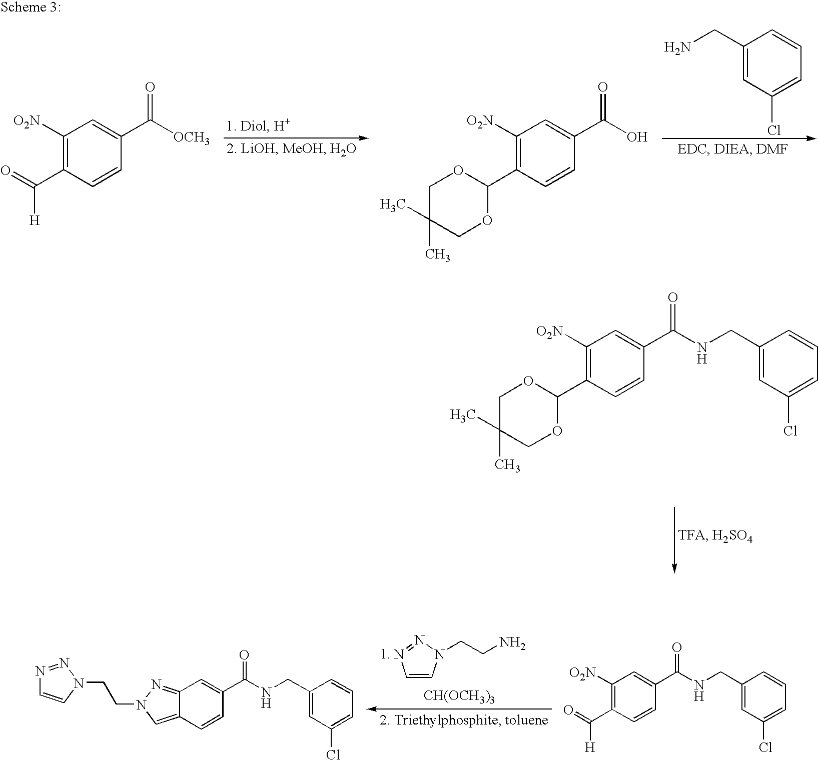

The compounds according to the invention display an unforeseeable, useful pharmacological and pharmacokinetic spectrum of action. They are compounds that exert an influence on the proteolytic activity of the serine protease thrombin. The compounds according to the invention inhibit the enzymatic cleavage of substrates that perform an essential role in the activation of coagulation and the aggregation of blood platelets.

They are therefore suitable for use as medicinal products for the treatment and/or prophylaxis of diseases in humans and animals.

The present invention also relates to the use of the compounds according to the invention for the treatment and/or prophylaxis of diseases, preferably of thromboembolic diseases and/or thromboembolic complications.

The “thromboembolic diseases” in the sense of the present invention include, in particular, diseases such as acute coronary syndrome (ACS), myocardial infarction with ST-segment elevation (STEMI) and without ST-segment elevation (non-STEMI), stable angina pectoris, unstable angina pectoris, reocclusions and restenoses after coronary interventions such as angioplasty, stent implantation or aortocoronary bypass, peripheral arterial occlusive diseases, pulmonary embolisms, venous thromboses, in particular in deep veins of the leg and renal veins, transient ischaemic attacks and thrombotic and thromboembolic stroke.

The compounds according to the invention are therefore also suitable for the prevention and treatment of cardiogenic thromboembolisms, such as cerebral ischaemias, stroke and systemic thromboembolisms and ischaemias, in patients with acute, intermittent or persistent cardiac arrhythmias, such as atrial fibrillation, and those that are subject to cardioversion, and moreover in patients with heart valve diseases or with artificial heart valves. Furthermore, the compounds according to the invention are suitable for the treatment of disseminated intravascular coagulation (DIC).

Thromboembolic complications also occur in microangiopathic haemolytic anaemias, extracorporeal blood circulation, such as haemodialysis, and heart valve prostheses.

Moreover, the compounds according to the invention can also be considered for exerting an influence on wound healing, for the prophylaxis and/or treatment of atherosclerotic vascular diseases and inflammatory diseases such as rheumatic diseases of the locomotor apparatus, coronary heart diseases, heart failure, hypertension, inflammatory diseases, e.g. asthma, inflammatory lung diseases, glomerulonephritis and inflammatory bowel diseases, as well as for the prophylaxis and/or treatment of Alzheimer's disease. In addition, the compounds according to the invention can be used for inhibition of tumour growth and formation of metastases, in microangiopathies, age-related macular degeneration, diabetic retinopathy, diabetic nephropathy and other microvascular diseases and for the prevention and treatment of thromboembolic complications, such as venous thromboembolisms, in tumour patients, in particular those undergoing major surgery or chemo- or radiotherapy.

The compounds according to the invention can furthermore also be used for the prevention of coagulation ex vivo, e.g. for the preservation of blood and plasma products, for the purification/pretreatment of catheters and other medical aids and equipment, for the coating of artificial surfaces of medical aids and equipment used in vivo or ex vivo or for biological samples containing blood platelets.

The present invention also relates to the use of the compounds according to the invention for the treatment and/or prophylaxis of diseases, in particular the aforementioned diseases.

The present invention also relates to the use of the compounds according to the invention for the production of a medicinal product for the treatment and/or prophylaxis of diseases, in particular the aforementioned diseases.

The present invention further relates to a method of treatment and/or prophylaxis of diseases, in particular the aforementioned diseases, using a therapeutically effective amount of a compound according to the invention.

The present invention further relates to medicinal products containing a compound according to the invention and one or more additional active substances.

The present invention further relates to a method of prevention of blood coagulation in vitro, in particular for banked blood or biological samples containing blood platelets, characterized in that an anticoagulation-effective amount of the compound according to the invention is added.

The present invention further relates to combinations of
A) compounds of formula (I) with
B) other pharmaceutical active substances, in particular with platelet inhibitors, anticoagulants, fibrinolytics, antilipaemics, coronary remedies and/or vasodilators.

“Combinations”, in the sense of the invention, mean not only dosage forms that contain all components (so-called fixed combinations), and combination packs that contain the components separated from one another, but also components that are applied simultaneously or with a time delay, provided they are used for the prophylaxis and/or treatment of the same disease. It is also possible to combine two or more active substances with one another, i.e. double or multiple combinations.

The individual actives of combinations are known from the literature and for the most part are commercially available.

Platelet inhibitors are for example acetylsalicylic acid (such as aspirin), ticlopidine (Ticlid) and clopidogrel (Plavix), or integrin antagonists such as glycoprotein-IIb/IIIa antagonists, for example abciximab, eptifibatide, tirofiban, lamifiban, lefradafiban and fradafiban.

Anticoagulation-effective substances (anticoagulants) are for example heparin (UFH), low-molecular heparins (LMH) such as tinzaparin, certoparin, parnaparin, nadroparin, ardeparin, enoxaparin, reviparin, dalteparin, danaparoid and factor Xa inhibitors.

Factor Xa inhibitors are for example:
Rivaroxaban (BAY 59-7939): 5-chloro-N-({(5S)-2-oxo-3-[4-(3-oxomorpholin-4-yl)phenyl]-1,3-oxazolidin-5-yl}methyl)thiophene-2-carboxamide [WO 2001/47919]

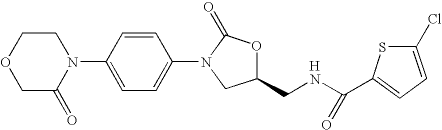

AX-1826 [S. Takehana et al. Japanese Journal of Pharmacology 2000, 82 (suppl. 1), 213P; T. Kayahara et al. Japanese Journal of Pharmacology 2000, 82 (suppl. 1), 213P]
Tanogitran (BIBT-986, prodrug: BIBT-1011): N-[(1R)-1-{2-[({4-amino(imino)methyl]-phenyl}amino)methyl]-1-methyl-1H-benzimidazol-5-yl}-1-methyl-2-oxo-2-pyrrolidin-1-ylethyl]glycine [American Chemical Society—226th National Meeting, New York City, N.Y., USA, 2003]

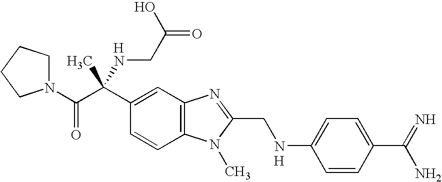

Compounds that were disclosed in WO 2004/056784.
YM-150 [Y. Iwatsuki et al. Blood 2006, 108, abstract 911 (ASH 2006)]
N-{4-Bromo-2-[(5-chloropyridin-2-yl)carbamoyl]-6-hydroxyphenyl}-1-isopropylpiperidine-4-carboxamide [JP 2005/179272]

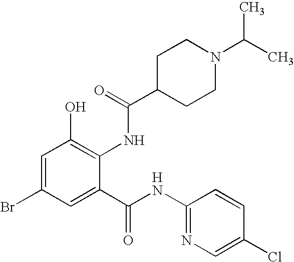

Compounds that were disclosed in WO 2000/242270.
AZ12300547: 6-[4-({2S)-4-[(3-Chloro-1H-indol-6-yl)sulphonyl]-2-methyl-6-oxopiperazin-1-yl}methyl)-phenyl]-2-methylpyridazin-3(2H)-one [K. L. Granberg et al. American Chemical Society—232nd National Meeting, San Francisco, USA, 2006, MEDI 391]

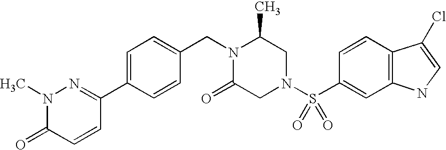

Compounds that were disclosed in WO 2007/008142.
Razaxaban (DPC-906): 1-(3-Amino-1,2-benzisoxazol-5-yl)-N-(4-{2-[(dimethylamino)-methyl]-1H-imidazol-1-yl}-2-fluorophenyl)-3-(trifluoromethyl)-1H-pyrazole-5-carboxamide [J. Med. Chem. 2005, 48, 1729-1744]

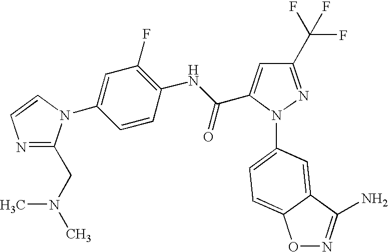

Apixaban (BMS-562247): 1-(4-Methoxyphenyl)-7-oxo-6-[4-(2-oxopiperidin-1-yl)phenyl]-4,5,6,7-tetrahydro-1H-pyrazolo[3,4-c]pyridine-3-carboxamide [WO 2003/026652, WO 2003/049681]

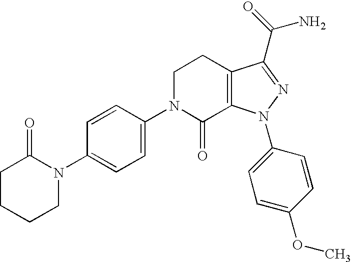

BMS-691648: 3-Chloro-N-[(3S.4R)-1-(methylsulphonyl)-4-{[4-(2-oxopyridin-1(2H)-yl)benzoyl]-amino}piperidin-3-yl]-1H-indole-6-carboxamide [T. Güngör et al. Drugs Fut. 2006, 31(Suppl A): abstract P118; WO 2004/082687]

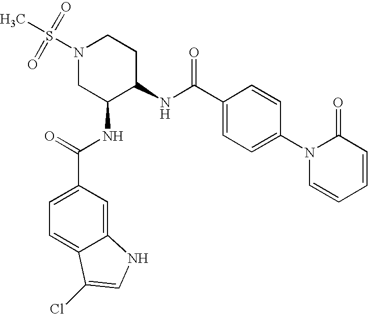

DX-9065a: (2S)-3-{7-[Amino(imino)methyl]-2-naphthyl}-2-(4-{[(3S)-1-ethanimidoyl-pyrrolidin-3-yl]oxy}phenyl)propionic acid [T. Nagahara et al. J. Med. Chem. 1994, 37, 1200-1207]

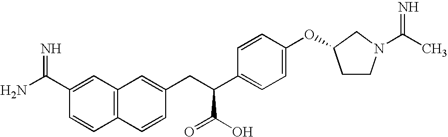

DU-176b [Y. Morishima et al. Blood 2004, 104, abstract 1862 (ASH 2004); T. Fukuda et al. Blood 2004, 104, abstract 1852 (ASH 2004); T. Furugohri et al. Blood 2004, 104, abstract 1851 (ASH 2004)]
N-(5-Chloropyridin-2-yl)-N′-[(1S,2R,4S)-4-(dimethylcarbamoyl)-2-{[(5-methyl-4,5,6,7-tetrahydro[1,3]thiazolo[5,4-c]pyridin-2-yl)carbonyl]amino}cyclohexyl]ethanediamide [US 2005/0020645, WO 2005/47296]

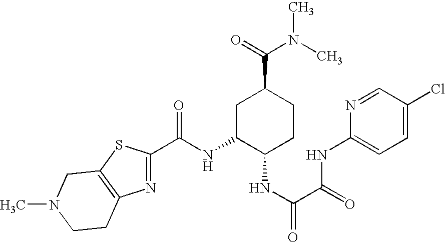

Compounds that were disclosed in US 2005/0020645.
LY517717: N-{(1R)-2-[4-(1-Methylpiperidin-4-yl)piperazin-1-yl]-2-oxo-1-phenylethyl}-1H-indole-6-carboxamide [WO 2000/76971, WO 2002/100847]

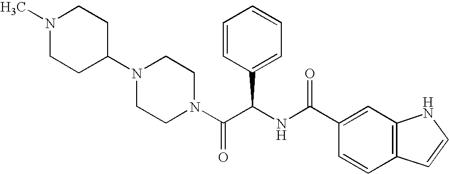

813893 [Proteinase Inhibitor Design—Fourth SCI-RSC Symposium, Proteinase 2004: Strategies for New Medicines (Part I), London]
6-Chloro-N-{(3S)-1-[(1S)-1-methyl-2-morpholin-4-yl-2-oxoethyl]-2-oxopyrrolidin-3-yl}naphthalene-2-sulphonamide [N. S. Watson et al. Bioorg. Med. Chem. Lett. 2006, 16, 3784; WO 2002/100830; WO 2002/100886]

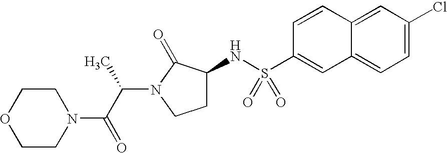

KFA-1982 (prodrug of KFA-1829) [T. Koizumi et al. Journal of Thrombosis and Hemostasis 2003, 1 Suppl 1, p 2022]
EMD-503982 [Merck KGaA Annual Report 2006, 48-49]
EMD-495235: 5-Chloro-N-[(1R)-1-(methoxymethyl)-2-{[3-methyl-4-(3-oxomorpholin-4-yl)-phenyl]amino}-2-oxoethyl]thiophene-2-carboxamide [Bioorg. Med. Chem. Lett. 2004, 14, 5817-5822]

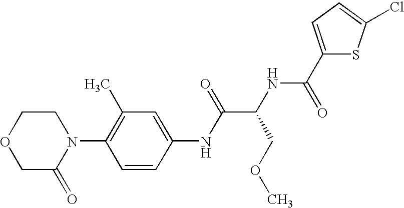

M-55113: 4-[(6-Chloro-2-naphthyl)sulphonyl]-1-[(1-pyridin-4-ylpiperidin-4-yl)methyl]piperazin-2-one [H. Nishida et al. Chem. Pharm. Bull. 2001, 49, 1237-1244]

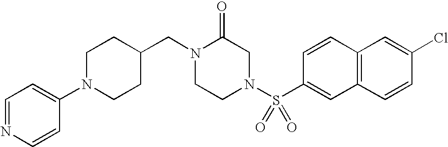

M-55551/M-55555: (2R)-4-[(6-Chloro-2-naphthyl)sulphonyl]-6-oxo-1-[(1-pyridin-4-ylpiperidin-4-yl)methyl]piperazine-2-carboxylic acid [H. Nishida et al. Chem. Pharm. Bull. 2002, 50, 1187-1194]

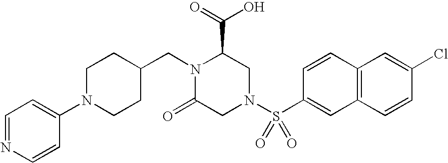

M-55190: (2R)-4-[(6-Chloro-2-naphthyl)sulphonyl]-6-oxo-1-[(1-pyridin-4-ylpiperidin-4-yl)-methyl]piperazine-2-carboxylic acid ethyl ester [H. Nishida et al. 16th Int Symp Med Chem, Bologna, 18-22 Sep. 2000, Abst PA-125]

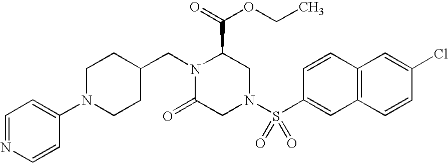

M-55532: 7-[(6-Chloro-2-naphthyl)sulphonyl]-8a-(methoxymethyl)-1′-pyridin-4-yltetrahydro-5H-spiro[1,3-oxazolo[3,2-a]pyrazine-2,4′-piperidin]-5-one [H. Nishida et al. 228th ACS National Meeting, Philadelphia, Aug. 22-26, 2004, MEDI-251; H. Nishida et al. Chem. Pharm. Bull. 2004, 52, 406-412; ditto 459-462]

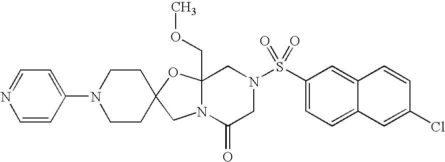

N-({7-[(5-Chloro-1H-indol-2-yl)sulphonyl]-5-oxo-1′-propionyltetrahydro-8aH-spiro[1,3-oxazolo-[3,2-a]pyrazine-2,4′-piperidin]-8a-yl}methyl)-N-methylglycine [WO 2006/106804]

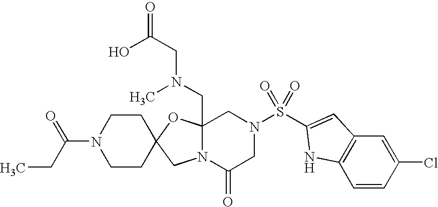

PRT54021 [U. Sinha et al. Blood 2006, 108, abstract 907 (ASH 2006); K. Abe et al. Blood 2006, 108, abstract 901 (ASH 2006)]
Compounds that were disclosed in WO 2006/002099.
Otamixaban (FXV-673, RPR-130673): (2R,3R)-2-{3-[Amino(imino)methyl]benzyl}-3-{[4-(1-oxidopyridin-4-yl)benzoyl]amino}butanoic acid methyl ester [V. Chu et al. Thrombosis Research 2001, 103, 309-324; K. R. Guertin et al. Bioorg Med. Chem. Lett. 2002, 12, 1671-1674]

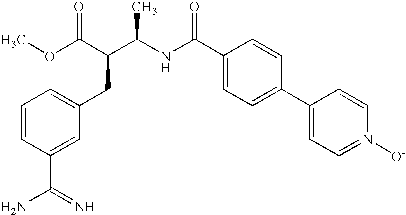

AVE3247 [Sanofi Aventis Company Presentation, Paris 2007, Feb. 13]
SAR377142 (SSR-7142) [Sanofi Aventis Company Presentation, Paris 2007, Feb. 13]
HMR-2906 [XVIIth Congress of the International Society for Thrombosis and Haemostasis, Washington D.C., USA, 14-21 Aug. 1999; Generating greater value from our products and pipeline. Aventis SA Company Presentation, 5 Feb. 2004]
Idraparinux [Harry R. Büller et al. Blood, 2006, 108, abstract 571 (ASH 2006)]
Fondaparinux

Plasminogen activators (thrombolytics/fibrinolytics) are for example tissue-plasminogen activator (t-PA), streptokinase, reteplase and urokinase.

Antilipaemics are in particular HMG-CoA-(3-hydroxy-3-methylglutaryl-coenzyme A)-reductase inhibitors such as lovastatin (Mevacor; U.S. Pat. No. 4,231,938), simvastatin (Zocor; U.S. Pat. No. 4,444,784), pravastatin (Pravachol; U.S. Pat. No. 4,346,227), fluvastatin (Lescol; U.S. Pat. No. 5,354,772) and atorvastatin (Lipitor; U.S. Pat. No. 5,273,995).

Coronary remedies/vasodilators are in particular ACE (angiotensin-converting-enzyme) inhibitors such as captopril, lisinopril, enalapril, ramipril, cilazapril, benazepril, fosinopril, quinapril and perindopril, or AII (angiotensin II) receptor antagonists such as embusartan (U.S. Pat. No. 5,863,930), losartan, valsartan, irbesartan, candesartan, eprosartan and temisartan, or β-adrenoceptor-antagonists such as carvedilol, alprenolol, bisoprolol, acebutolol, atenolol, betaxolol, carteolol, metoprolol, nadolol, penbutolol, pindolol, propanolol and timolol, or alpha-1-adrenoceptor-antagonists such as prazosin, bunazosin, doxazosin and terazosin, or diuretics such as hydrochlorothiazide, furosemide, bumetanide, piretanide, torsemide, amiloride and dihydralazine, or calcium channel blockers such as verapamil and diltiazem, or dihydropyridine derivatives such as nifedipine (Adalat) and nitrendipine (Bayotensin), or nitro preparations such as isosorbide-5-mononitrate, isosorbide-dinitrate and glyceroltrinitrate, or substances that produce an increase in cyclic guanosine monophosphate (cGMP), such as stimulators of soluble guanylate cyclase (WO 98/16223, WO 98/16507, WO 98/23619, WO 00/06567, WO 00/06568, WO 00/06569, WO 00/21954, WO 00/66582, WO 01/17998, WO 01/19776, WO 01/19355, WO 01/19780, WO 01/19778, WO 07/045366, WO 07/045367, WO 07/045369, WO 07/045370, WO 07/045433).

The compounds according to the invention can act systemically and/or locally. For this purpose they can be applied by a suitable route, e.g. oral, parenteral, pulmonary, nasal, sublingual, lingual, buccal, rectal, dermal, transdermal, conjunctival, otic or as implant or stent.

For these routes of administration, the compounds according to the invention can be administered in suitable dosage forms.

Dosage forms functioning according to the prior art and providing rapid and/or modified release of the compounds according to the invention, and containing the compounds according to the invention in crystalline and/or amorphized and/or dissolved form, e.g. tablets (uncoated or coated tablets, for example enteric-coated or with slow-dissolving or insoluble coatings, which control the release of the compound according to the invention), tablets that disintegrate quickly in the oral cavity or films/wafers, films/lyophilizates, capsules (for example hard or soft gelatin capsules), sugar-coated pills, granules, pellets, powders, emulsions, suspensions, aerosols or solutions, are suitable for oral application.

Parenteral application can take place with avoidance of an absorption step (e.g. by intravenous, intraarterial, intracardial, intraspinal or intralumbal application) or including absorption (e.g. intramuscular, subcutaneous, intracutaneous, percutaneous or intraperitoneal application). Suitable dosage forms for parenteral application are, among others, preparations for injection and infusion in the form of solutions, suspensions, emulsions, lyophilizates or sterile powders.

Oral application is preferred.

For example, inhaled pharmaceutical forms (including powder inhalers, nebulizers), nasal drops, solutions, sprays; tablets for lingual, sublingual or buccal application, films/wafers or capsules, suppositories, ear or eye preparations, vaginal capsules, aqueous suspensions (lotions, shaking mixtures), lipophilic suspensions, ointments, creams, transdermal therapeutic systems (such as patches), milk, pastes, foams, dusting powders, implants or stents, are suitable for the other routes of administration.

The compounds according to the invention can be converted to the aforementioned dosage forms. This can be carried out in an already known manner by mixing with inert, nontoxic, pharmaceutically suitable excipients. These excipients include, among others, carriers (for example microcrystalline cellulose, lactose, mannitol), solvents (e.g. liquid polyethylene glycols), emulsifiers and dispersants or wetting agents (for example sodium dodecylsulphate, polyoxysorbitanoleate), binders (for example polyvinylpyrrolidone), synthetic and natural polymers (for example albumin), stabilizers (e.g. antioxidants such as ascorbic acid), colourants (e.g. inorganic pigments such as iron oxides) and taste and/or odour correctives.

The present invention further relates to medicinal products that contain at least one compound according to the invention, preferably together with one or more inert nontoxic, pharmaceutically suitable excipients, and their use for the aforementioned purposes.

In general it has proved advantageous, in parenteral application, to administer amounts of about 5 to 250 mg per 24 hours for achieving effective results. In oral application, the amount is about 5 to 100 mg per 24 hours.

Nevertheless, it may possibly be necessary to deviate from the stated amounts, namely according to body weight, route of administration, individual response to the active substance, type of preparation and point of time or interval in which application is carried out.

The percentages in the following tests and examples are, unless stated otherwise, percentages by weight; parts are parts by weight. Proportions of solvents, dilutions and concentrations of liquid/liquid solutions relate in each case to the volume. The abbreviation “w/v” means “weight/volume”. For example, “10% w/v”: 100 ml of solution or suspension contains 10 g of substance.

### A) EXAMPLES

Abbreviations:

abs. absolute

Boc tert-butoxycarbonyl

CDCl₃ deuterochloroform

CO₂ carbon dioxide

d day

DCM dichloromethane

DIEA N,N-diisopropylethylamine

DMAP 4-N,N-dimethylaminopyridine

DMF dimethylformamide

DMSO dimethylsulphoxide

of th. of theoretical

EDC N′-(3-dimethylaminopropyl)-N-ethylcarbodiimide×HCl

eq. equivalent

ESI electrospray ionization (in MS)

ges. saturated

h hour

HOBt 1-hydroxy-1H-benzotriazole×H₂O

HPLC high-pressure/high-performance liquid chromatography

conc. concentrated

LC-MS liquid chromatography/mass spectrometry

min minutes

MS mass spectrometry

MW molecular weight [g/mol]

NMR nuclear magnetic resonance spectroscopy

PyBOP 1-benzotriazolyloxy-tripyrrolidinophosphonium hexafluorophosphate

R_{f} retention index (in TLC)

RP-HPLC reversed-phase HPLC

RT room temperature

Rₜ retention time (in HPLC)

TBTU (benzotriazol-1-yloxy)bisdimethylaminomethylium fluoroborate

TFA trifluoracetic acid

THF tetrahydrofuran

LC-MS Methods:

Method 1: Equipment type MS: Micromass ZQ; equipment type HPLC: HP 1100 series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; furnace: 50° C.; UV detection: 210 nm.

Method 2: Instrument: Micromass Quattro LCZ with HPLC Agilent Series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; furnace: 50° C.; UV detection: 208-400 nm.

Method 3: Equipment type MS: Micromass ZQ; equipment type HPLC: Waters Alliance 2795; column: Merck Chromolith SpeedROD RP-18e 100 mm×4.6 mm; eluent A: water+0.5 ml 50% formic acid/l; eluent B: acetonitrile+0.5 ml 50% formic acid/l; gradient: 0.0 min 10% B→7.0 min 95% B→9.0 min 95% B; flow: 0.0 min 1.0 ml/min→7.0 min 2.0 ml/min→9.0 min 2.0 ml/min; furnace: 35° C.; UV detection: 210 nm.

Method 4: Equipment type MS: Micromass ZQ; equipment type HPLC: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; furnace: 50° C.; UV detection: 210 nm.

Method 5: Equipment type MS: Micromass ZQ; equipment type HPLC: HP 1100 series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min. 2 ml/min; furnace: 50° C.; UV detection: 210 nm.

Method 6: Instrument: Micromass Platform LCZ with HPLC Agilent Series 1100; column: Thermo Hypersil GOLD 3μ 20 mm×4 mm; eluent A: 1 l water+0.5 ml 50% formic acid, eluent B: 1 l acetonitrile+0.5 ml 50% formic acid; gradient: 0.0 min 100% A→0.2 min 100% A→2.9 min 30% A→3.1 min 10% A→5.5 min 10% A; furnace: 50° C.; flow: 0.8 ml/min; UV detection: 210 nm.

Method 7: Instrument: Micromass Quattro LCZ with HPLC Agilent Series 1100; column: Phenomenex Onyx Monolithic C18, 100 mm×3 mm Eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow: 2 ml/min; furnace: 40° C.; UV detection: 208-400 nm.

Method 8: Instrument: Micromass Quattro LCZ with HPLC Agilent Series 1100; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; furnace: 50° C.; UV detection: 208-400 nm.

Enantiomer separation: Enantiomer separation of corresponding examples of application can be achieved using a Daicel Chiralpak AD-H, 5 μM 250 mm×20 mm column with a solvent system of iso-hexane and ethanol and diethylamine addition.

Starting Compounds

### Example 1A

### Methyl-1H-indazole-6-carboxylate

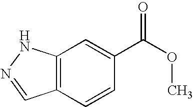

In a 500-ml three-necked flask with mechanical stirrer, 20 g (121 mmol) of 3-amino-4-methylbenzoic acid methyl ester, 17.14 g ammonium tetrafluoroborate and 217 ml water are cooled to 0° C. and then 24.6 ml concentrated hydrochloric acid is added dropwise. Then a solution of 8.35 g (121.1 mmol) of sodium nitrite in 21.7 ml water is added dropwise within 20 min and stirring is continued for 40 min at 3° C. It is filtered with suction through a frit and the filter cake is mixed with methanol (100 ml), dried and then mixed with methyl-tert.-butyl ether and dried again. After vacuum drying we obtain 26.99 g (84% of th.) of the diazonium tetrafluoroborate salt, which is used further without further purification. For the production of the corresponding indazole derivative, 26.99 g of the diazonium salt (102.2 mmol) is suspended in 500 ml dichloromethane in a 1 l round-bottom flask and 1.43 g (5.4 mmol) of 18-crown-6-ether and 22.8 g (232.1 mmol) of potassium acetate are added at RT and stirred for 3 h at RT. 100 ml water is added to the suspension, the dichloromethane phase is removed and the aqueous phase is extracted once more with dichloromethane. The combined organic phases are washed with 50 ml water and dried over sodium sulphate. Chromatography on silica gel with cyclohexane/ethyl acetate gives 17.54 g (97% of th.) of the product as a solid.

LCMS (method 1): Rₜ=1.41 min (m/z=177 (M+H)⁺)

¹H-NMR (400 MHz, DMSO-d₆): δ=13.95 (s, 1H), 8.18 (d, 2H), 7.89 (d, 1H), 7.68 (d, 1H), 3.90 (s, 3H).

### Example 2A

### N-(3-Chlorobenzyl)-1H-indazole-6-carboxamide

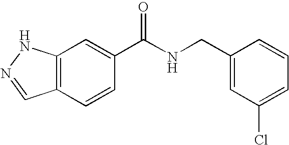

2.95 g (16.76 mmol) of methyl-1H-indazole-6-carboxylate (Example 1A) and 2.61 g (18.44 mmol) 3-chlorobenzylamine are put in a mixture of 54 ml dichloromethane and 54 ml toluene. A 10% solution of methylaluminoxane in toluene is slowly added dropwise. An exothermic reaction takes place. It is stirred for 16 h at RT and then another equivalent of 3-chlorobenzylamine is added and it is stirred at 40° C. for a further 16 h. The raw preparation is poured onto a mixture of ice/2N hydrochloric acid and is extracted at pH 4 with ethyl acetate. After removing the solvent we obtain 4.36 g (83% of th.) of the product as a solid.

LCMS (method 4): Rₜ=2.08 min (m/z=286 (M+H)⁺)

¹H-NMR (300 MHz, DMSO-d₆): δ=13.36 (s, 1H), 9.18 (t, 1H), 8.12 (d, 2H), 7.84 (d, 1H), 7.63 (d, 1H), 7.25-7.41 (m, 4H), 4.51 (d, 2H).

### Example 3A

### Methyl 4-(5,5-dimethyl-1,3-dioxan-2-yl)-3-nitrobenzoate

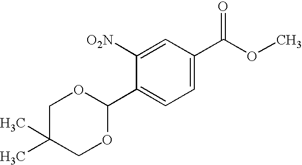

7 g (33.5 mmol) of methyl-4-formyl-3-nitrobenzoate, 69.7 g (0.67 mol) 2,2-dimethyl-1,3-propanediol and 576 mg (3.35 mmol) 4-toluenesulphonic acid are heated in 300 ml toluene for 18 h with a water trap. The solvent is reduced, saturated aqueous ammonium chloride solution is added and it is then extracted three times with ethyl acetate. The combined organic phases are dried over sodium sulphate and, after removing the solvent, the solid obtained is used without further purification.

MS (DCI, NH₃): m/z=313 (M+NH₄)⁺.

¹H-NMR (400 MHz, DMSO-d₆): δ=8.35 (d, 1H), 8.28 (dd, 1H), 7.97 (d, 1H), 5.93 (s, 1H), 3.91 (s, 3H), 3.69 (s, 4H), 1.13 (s, 3H), 0.75 (s, 3H).

### Example 4A

### 4-(5,5-Dimethyl-1,3-dioxan-2-yl)-3-nitrobenzoic acid

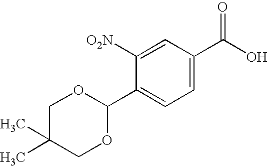

9.88 g (33.47 mmol) of the ester from Example 3A is dissolved in a mixture of 180 ml methanol and 60 ml water, 3.27 g (0.134 mol) lithium hydroxide is added and it is stirred for 16 h at RT. The pH is adjusted to pH 6 with hydrochloric acid and it is extracted three times with ethyl acetate. The combined organic phases are dried over sodium sulphate and, after removing the solvent, the solid obtained (9.4 g, 99% of th.) is reacted without further purification.

LCMS (method 4): Rₜ=2.28 min (m/z=282 (M+H)⁺)

¹H-NMR (400 MHz, DMSO-d₆): δ=8.23 (d, 1H), 8.16 (dd, 1H), 7.79 (d, 1H), 7.38 (s, 1H, broad), 5.89 (s, 1H), 3.67 (s, 4H), 1.14 (s, 3H), 0.75 (s, 3H).

### Example 5A

### N-(3-Chlorobenzyl)-4-(5,5-dimethyl-1,3-dioxan-2-yl)-3-nitrobenzamide

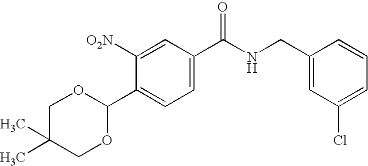

12 g (42.66 mmol) of the acid from Example 4A is put in 250 ml dichloromethane and 30 ml DMF, 7.82 g (64 mmol) DMAP, 16.54 g (128 mmol) DIEA, 16.36 g (85.33 mmol) EDC and finally 6.65 g (46.93 mmol) 3-chlorobenzylamine are added and it is stirred for 16 h at RT. Water is added and it is extracted three times with ethyl acetate. The combined organic phases are dried over sodium sulphate and, after removing the solvent, the solid obtained is chromatographed on silica gel (dichloromethane/ethanol). We obtain 9.3 g (54% of th.) of the product as a solid.

LCMS (method 4): Rₜ=2.63 min (m/z=405 (M+H)⁺)

¹H-NMR (400 MHz, DMSO-d₆): δ=9.39 (t, 1H), 8.39 (d, 1H), 8.24 (dd, 1H), 7.92 (d, 1H), 7.28-7.41 (m, 4H), 5.92 (s, 1H), 4.50 (d, 2H), 3.69 (s, 4H), 1.14 (s, 3H), 0.76 (s, 3H).

### Example 6A

### N-(3-Chlorobenzyl)-4-formyl-3-nitrobenzamide

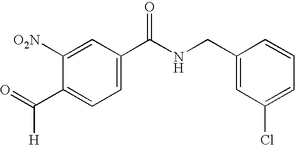

4.21 g (8.53 mmol) of the acetal from Example 5A is put in a mixture of 115 ml trifluoracetic acid and 42 ml 10% sulphuric acid and stirred for 4 h at RT. The mixture is poured into ice water and extracted three times with dichloromethane. The organic phases are washed once with saturated aqueous sodium hydrogencarbonate solution and then the solvent is removed. We obtain 3.2 g (85% of th.) of the aldehyde.

LCMS (method 4): Rₜ=2.15 min (m/z=319 (M+H)⁺)

¹H-NMR (400 MHz, DMSO-d₆): δ=10.30 (s, 1H), 9.55 (t, 1H), 8.61 (s, 1H), 8.37 (d, 1H), 8.01 (d, 1H), 7.29-7.43 (m, 4H), 4.53 (d, 2H).

### Example 7A

### 3-Amino-4-methyl-5-nitrobenzoic acid

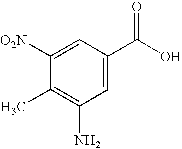

20 g (88.44 mmol) of 4-methyl-3,5-dinitrobenzoic acid is suspended in a mixture of 500 ml methanol and 125 ml water and 53.1 g (265.3 mmol) sodium dithionite is added in portions. Then it is stirred for 16 h at RT. The precipitate is filtered off with suction. The methanol is distilled from the filtrate and then the aqueous phase is extracted with ethyl acetate five times. After approx. 200 ml semiconcentrated hydrochloric acid has been added, the aqueous phase is heated under reflux for 16 h. After cooling, it is extracted four times with ethyl acetate and the combined ethyl acetate phases are dried over magnesium sulphate. We obtain 13.96 g (80% of th.) of the acid as a solid.

LCMS (method 4): Rₜ=1.32 min (m/z=197 (M+H)⁺)

¹H-NMR (400 MHz, DMSO-d₆): δ=13.13 (s, 1H, broad), 7.49 (s, 1H), 7.42 (s, 1H), 5.83 (s, 2H), 2.14 (s, 3H).

### Example 8A

### 3-Chloro-4-methyl-5-nitrobenzoic acid

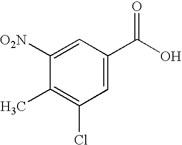

4.73 g (45.8 mmol) tert.-butylnitrite and 4.94 g (36.7 mmol) copper(II) chloride are put in 120 ml acetonitrile and 6 g (30.59 mmol) of the aniline from Example 7A is added in portions over a period of 5 min. The mixture is heated to 65° C. in 10 min and, after cooling, 500 ml of 6N hydrochloric acid is added. It is extracted with ethyl acetate several times, the combined organic phases are washed with saturated sodium chloride solution and dried over magnesium sulphate. After removing the solvent we obtain 5.9 g (79% of th.) of the chloro-aromatic as a solid, which is used without further purification.

MS (ES-): m/z=214 (M−H)⁺.

¹H-NMR (400 MHz, DMSO-d₆): δ=13.82 (s, 1H, broad), 8.30 (s, 1H), 8.20 (s, 1H), 2.54 (s, 3H).

### Example 9A

### 3-Chloro-N-(3-chlorobenzyl)-4-methyl-5-nitrobenzamide

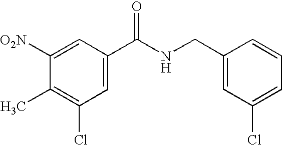

7 g (32.47 mmol) of the acid from Example 8A is suspended in 340 ml dichloromethane and 5.98 g (42.21 mmol) 3-chlorobenzylamine, 5.95 g (48.7 mmol) DMAP, 12.59 g (97.41 mmol) DIEA and 12.45 g (64.94 mmol) EDC are added. The suspension is stirred for 15 h at RT. 2M hydrochloric acid is added, and it is extracted three times with ethyl acetate. The combined organic phases are washed with saturated aqueous sodium chloride solution and then dried over magnesium sulphate. After removing the solvent we obtain 10.57 g (74% of th.) of the product as a solid, which is used without further purification.

LCMS (method 4): Rₜ=2.54 min (m/z=339 (M+H)⁺)

¹H-NMR (400 MHz, DMSO-d₆): δ=9.41 (t, 1H), 8.38 (d, 1H), 8.30 (d, 1H), 7.27-7.42 (m, 4H), 4.50 (d, 2H), 2.54 (s, 3H).

### Example 10A

### 3-Amino-5-chloro-N-(3-chlorobenzyl)-4-methylbenzamide

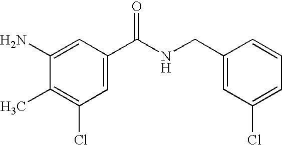

10.4 g (30.66 mmol) of the nitro compound from Example 9A is dissolved in 260 ml ethanol and 23.56 g (122.65 mmol) tin(II) chloride is added. It is heated for 16 h under reflux, cooled, ethyl acetate is added and it is made basic with 20% sodium hydroxide solution. The precipitate is filtered off with suction, washed with ethyl acetate several times and the combined organic phases are concentrated by evaporation. After chromatography with cyclohexane/ethyl acetate on silica gel we obtain 6.48 g (68% of th.) of the aniline as a solid.

LCMS (method 4): Rₜ=2.14 min (m/z=309 (M+H)⁺)

¹H-NMR (400 MHz, DMSO-d₆): δ=8.92 (t, 1H), 7.22-7.39 (m, 4H), 7.11 (d, 2H), 5.41 (s, 2H), 4.41 (d, 2H), 2.14 (s, 3H).

### Example 11A

### 4-Chloro-N-(3-chlorobenzyl)-1H-indazole-6-carboxamide

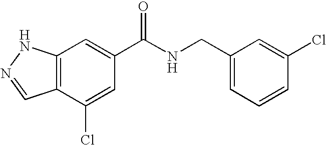

Similarly to the preparation procedure in Example 1A, 1 g (3.23 mmol) of the aniline from Example 10A is diazotized with 0.67 g (9.7 mmol) sodium nitrite in hydrochloric acid and then cyclized to the corresponding indazole derivative. We obtain, after purification by prep. HPLC, 640 mg (79% of th.) of product as a solid, which crystallizes from acetonitrile/water.

LCMS (method 4): Rₜ=2.07 min (m/z=320 (M+H)⁺)

¹H-NMR (400 MHz, DMSO-d₆): δ=13.77 (s, 1H), 9.30 (t, 1H), 8.22 (s, 1H), 8.10 (s, 1H), 7.70 (s, 1H), 7.29-7.41 (m, 4H), 4.51 (d, 2H).

### Example 12A

### Methyl-2-[2-(1H-pyrazol-1-yl)ethyl]-2H-indazole-6-carboxylate

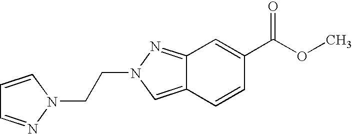

Similarly to the preparation procedure in Example 6, 1 g (4.64 mmol) of methyl-4-formyl-3-nitrobenzoate is cyclized with 515.5 mg (4.64 mmol) of 2-(1H-pyrazol-1-yl)ethanamine to the corresponding indazole derivative. We obtain, after purification by prep. HPLC, 528 mg (42% of th.) of product.

MS (DCI, NH₃): m/z=271 (M+H)⁺.

¹H-NMR (400 MHz, DMSO-d₆): δ=8.29 (s, 1H), 8.19 (s, 1H), 7.76 (d, 1H), 7.53 (d, 1H), 7.44 (s, 2H), 6.12 (t, 1H), 4.92 (t, 2H), 4.72 (t, 2H), 3.88 (s, 3H).

### Example 13A

### 2-[2-(1H-Pyrazol-1-yl)ethyl]-2H-indazole-6-carboxylic acid

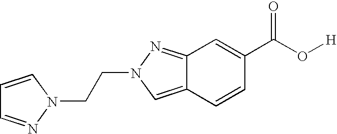

528 mg (1.95 mmol) of the ester from Example 12A is dissolved in a mixture of 60 ml methanol and 20 ml water and 140 mg (5.86 mol) lithium hydroxide is added and it is stirred for 3 h at 50° C. The pH is adjusted to pH 6 with hydrochloric acid and it is extracted three times with ethyl acetate. The combined organic phases are dried over sodium sulphate and, after removing the solvent, the solid obtained (468 mg, 92% of th.) is reacted without further purification.

LCMS (method 2): Rₜ=1.42 min (m/z=257.2 (M+H)⁺)

¹H-NMR (400 MHz, DMSO-d₆): δ=12.89 (s, broad, 1H), 8.26 (s, 1H), 8.16 (s, 1H), 7.72 (d, 1H), 7.52 (d, 1H), 7.43 (s, 2H), 6.12 (t, 1H), 4.91 (t, 2H), 4.72 (t, 2H).

### Example 14A

### Methyl-3-methoxy-4-methyl-5-nitrobenzoate

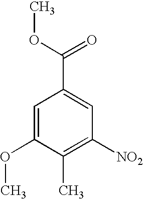

For the preparation of the ester see: M. Harris, et al., J. Am. Chem. Soc. 1979, 101, 437.

### Example 15A

### 3-Methoxy-4-methyl-5-nitrobenzoic acid

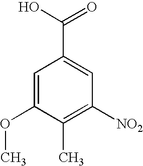

1.63 g (7.25 mmol) of methyl-3-methoxy-4-methyl-5-nitrobenzoate (Example 14A) is dissolved in a mixture of 30 ml methanol and 10 ml water and 708.8 mg (29 mmol) lithium hydroxide is added. It is stirred for 16 h at RT, then acidified with 2N hydrochloric acid and extracted three times with ethyl acetate. The combined organic phases are dried over sodium sulphate and then the solvent is removed. We obtain 1.49 g of product (86% of th.) as a solid.

LCMS (method 2): Rₜ=2.00 min (m/z=210 (M−H)⁺)

¹H-NMR (400 MHz, DMSO-d₆): δ=13.56 (s, 1H, broad), 7.95 (s, 1H), 7.72 (s, 1H), 3.96 (s, 3H), 2.02 (s, 3H).

### Example 16A

### N-(3-Chlorobenzyl)-3-methoxy-4-methyl-5-nitrobenzamide

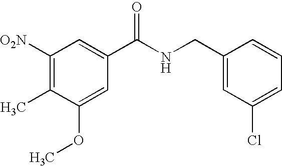

Similarly to the preparation procedure in Example 9A, 1.47 g (6.96 mmol) of the acid from Example 15A is reacted with 1.08 g (7.66 mmol) 3-chlorobenzylamine to the corresponding amide. After working up, we obtain 2.12 g (73% of th.) of product as a solid.

LCMS (method 2): Rₜ=2.61 min (m/z=333 (M−H)⁺)

### Example 17A

### 3-Amino-N-(3-chlorobenzyl)-5-methoxy-4-methylbenzamide

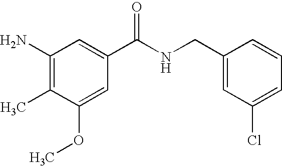

Similarly to the preparation procedure in Example 10A, 2.12 g (6.33 mmol) of the nitro compound from Example 16A is reduced with 4.8 g (25.3 mmol) tin(II) chloride to the corresponding aniline derivative. We obtain, after purification on silica gel, 1.56 g (81% of th.) of product as resin.

LCMS (method 2): Rₜ=2.07 min (m/z=305 (M+H)⁺)

¹H-NMR (400 MHz, DMSO-d₆): δ=8.87 (t, 1H), 7.20-7.40 (m, 4H), 6.93 (s, 1H), 6.82 (s, 1H), 4.44 (d, 2H), 3.78 (s, 2H), 3.52 (s, 3H), 1.96 (s, 3H).

### Example 18A

### N-(3-Chlorobenzyl)-4-methoxy-1H-indazole-6-carboxamide

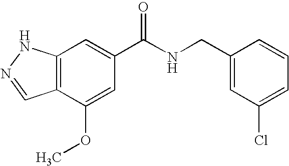

329 mg (1.1 mmol) of 3-amino-N-(3-chlorobenzyl)-5-methoxy-4-methylbenzamide (Example 17A) is put at −10° C. in 8 ml THF and, successively, 0.274 ml (2.16 mmol) bortrifluoride-etherate and 0.19 g (1.62 mmol) isoamyl nitrite, dissolved in 0.7 ml THF, are slowly added dropwise. Then it is stirred for 30 min at this temperature. Diethyl ether is added, it is stirred for 15 min and the precipitate is filtered off with suction. This is taken up in 9 ml dichloromethane and 0.015 g (0.057 mmol) 18-crown-6-ether and 0.241 g (2.45 mmol) potassium acetate are added. It is stirred for 15 h at RT and, after purification by prep. HPLC, we obtain 65 mg (18% of th.) of product as a solid.

LCMS (method 2): Rₜ=2.06 min (m/z=314 (M−H)⁺)

¹H-NMR (400 MHz, DMSO-d₆): δ=13.43 (s, 1H), 9.18 (t, 1H), 8.09 (s, 1H), 7.70 (s, 1H), 7.25-7.41 (m, 4H), 7.05 (s, 1H), 4.51 (d, 2H), 3.97 (s, 3H).

### Example 19A

### 3-Cyano-4-methyl-5-nitrobenzoic acid

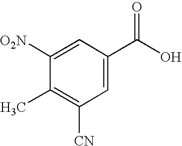

1.1 g (12.24 mmol) copper cyanide is suspended in 9 ml water and 1.7 g (34.67 mmol) sodium cyanide is added and it is stirred at 40° C. for 30 min. A solution of 0.89 g (12.95 mmol) sodium nitrite in 2.8 ml water is slowly added dropwise at 0° C., while stirring, to a suspension of 2 g (10.20 mmol) of 3-amino-4-methyl-5-nitrobenzoic acid (Example 7A) in 18.5 ml water and 3 ml conc. hydrochloric acid, keeping the temperature below 5° C. Then this solution is poured into a dropping funnel cooled with ice water and slowly added dropwise to the sodium cyanide/copper cyanide solution. It is stirred for 4 h at RT (evolution of gas). It is extracted with ethyl acetate several times, the combined organic phases are washed with saturated sodium chloride solution and dried over magnesium sulphate. We obtain, after removing the solvent, 1.75 g (69% of th.) of the title compound as a solid, which is used without further purification.

MS (ES-): m/z=205 (M−H)⁺.

¹H-NMR (400 MHz, DMSO-d₆): δ=14.01 (s, 1H, broad), 8.62 (s, 1H), 8.55 (s, 1H), 2.71 (s, 3H).

### Example 20A

### N-(3-Chlorobenzyl)-3-cyano-4-methyl-5-nitrobenzamide

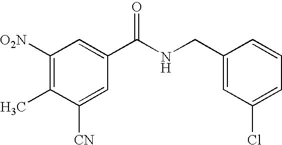

Similarly to the preparation procedure in Example 9A, 1.73 g (8.392 mmol) of the acid from Example 19A is reacted with 1.31 g (9.231 mmol) 3-chlorobenzylamine to the corresponding amide. After working up, we obtain 2.76 g of product (76% of th.) as a solid.

LCMS (method 4): Rₜ=2.31 min (m/z=330 (M+H)⁺)

¹H-NMR (400 MHz, DMSO-d₆): δ=9.47 (t, 1H), 8.71 (d, 1H), 8.63 (d, 1H), 7.27-7.43 (m, 4H), 4.51 (d, 2H), 2.71 (s, 3H).

### Example 21A

### 3-Amino-N-(3-chlorobenzyl)-5-cyano-4-methylbenzamide

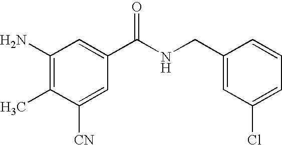

Similarly to the preparation procedure in Example 10A, 1.38 g (4.185 mmol) of the nitro compound (Example 20A) is reduced with 3.17 g (16.74 mmol) tin(II) chloride to the corresponding aniline derivative. We obtain, after purification on silica gel, 1.13 g (90% of th.) as a solid.

LCMS (method 2): Rₜ=2.15 min (m/z=300 (M+H)⁺)

¹H-NMR (400 MHz, DMSO-d₆): δ=9.03 (t, 1H), 7.43 (s, 1H), 7.39 (s, 1H), 7.23-7.38 (m, 4H), 5.65 (s, 2H), 4.43 (d, 2H), 2.26 (s, 3H).

### Example 22A

### N-(3-Chlorobenzyl)-4-cyano-1H-indazole-6-carboxamide

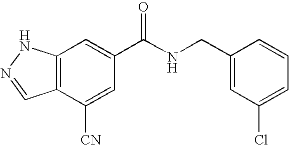

Similarly to the preparation in Example 18A, 170 mg (21% of th.) of the indazole derivative is isolated as a solid starting from 0.5 g (1.668 mmol) of 3-amino-N-(3-chlorobenzyl)-5-cyano-4-methylbenzamide (Example 21A).

LCMS (method 2): Rₜ=2.10 min (m/z=309 (M−H)⁺)

¹H-NMR (400 MHz, DMSO-d₆): δ=14.04 (s, 1H), 9.39 (t, 1H), 8.45 (s, 1H), 8.40 (s, 1H), 8.20 (s, 1H), 7.28-7.43 (m, 4H), 4.53 (d, 2H).

### Example 23A

### 2-Pyridin-2-yl-3-(tetrahydro-2H-pyran-4-yl)propanenitrile

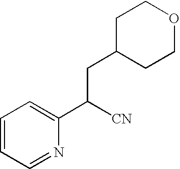

750 mg (6.35 mmol) 2-pyridylactonitrile and 69.13 mg (0.254 mmol) benzyltriethylammonium bromide are put in 10 ml 25% sodium hydroxide solution, then 1.35 g (7.56 mmol) 4-(bromomethyl)tetrahydro-2H-pyrane is added, and it is stirred for 15 h at RT. After aqueous processing and extraction with ethyl acetate, the raw product is purified by prep HPLC. We obtain 401 mg (29% of th.) of the product as oil.

LCMS (method 6): Rₜ=1.57 min (m/z=217 (M+H)⁺)

¹H-NMR (400 MHz, DMSO-d₆): δ=8.59 (d, 1H), 7.85 (dt, 1H), 7.48 (d, 1H), 7.38 (dd, 1H), 4.43 (dd, 1H), 3.82 (dd, 2H), 3.20-3.30 (m, 2H), 1.88-1.97 (m, 1H), 1.77-1.86 (m, 1H), 1.52-1.70 (m, 3H), 1.14-1.27 (m, 2H).

### Example 24A

### 2-Pyridin-2-yl-3-(tetrahydro-2H-pyran-4-yl)propan-1-amine

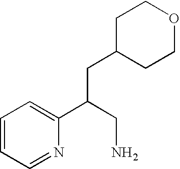

400 mg (1.85 mmol) 2-pyridin-2-yl-3-(tetrahydro-2H-pyran-4-yl)propanenitrile (Example 23A) is dissolved in 15 ml methanol and, at 0° C., 880 mg (3.70 mmol) cobalt(II) chloride hexahydrate and then 749 mg (19.79 mmol) sodium boron hydride are added. It is stirred for 30 min at 0° C. and then allowed to reach RT. After approx. 1 h, 2N hydrochloric acid is added until the precipitate has dissolved and it is then made basic with conc. ammonia solution. The precipitate is filtered off. After removing the solvent we obtain 317 mg (78% of th.) of product as a solid, which is used without further purification.

LCMS (method 6): Rₜ=2.13 min (m/z=221 (M+H)⁺)

### Example 25A

### Methyl-2-(2-ethylbutyl)-2H-indazole-6-carboxylate

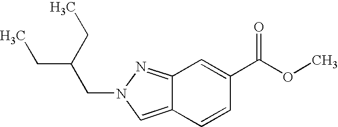

Similarly to the preparation procedure in Example 6, the corresponding indazole derivative is prepared from methyl-4-formyl-3-nitrobenzoate with 2-ethylbutan-1-amine.

LCMS (method 1): Rₜ=2.65 min (m/z=261 (M+H)⁺)

¹H-NMR (400 MHz, DMSO-d₆): δ=8.51 (s, 1H), 8.29 (s, 1H), 7.81 (d, 1H), 7.56 (dd, 1H), 4.38 (d, 2H), 3.88 (s, 3H), 1.98 (pent, 1H), 1.24 (pent, 4H), 0.86 (t, 6H).

### Example 26A

### 2-(2-Ethylbutyl)-2H-indazole-6-carboxylic acid

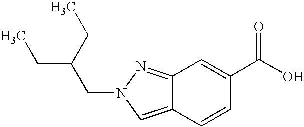

Similarly to the preparation procedure in Example 4A, the ester from Example 25A is saponified. We obtain the product as a solid at a yield of 95% of th.

LCMS (method 1): Rₜ=2.23 min (m/z=247 (M+H)⁺)

¹H-NMR (400 MHz, DMSO-d₆): δ=12.85 (s, 1H, broad), 8.49 (s, 1H), 8.26 (s, 1H), 7.77 (d, 1H), 4.55 (dd, 1H), 4.37 (d, 2H), 1.98 (pent, 1H), 1.24 (pent, 4H), 0.86 (t, 6H).

### Example 27A

### 2-[(2-Hydroxyethyl)amino]-3-phenylpropanenitrile

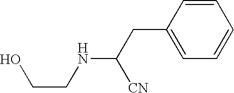

1000 mg (8.32 mmol) phenylacetaldehyde is put in 35 ml dichloromethane; 533.8 mg (8.74 mmol) 2-aminoethanol and 1 g 4A molecular sieve are added. It is stirred at RT for 1.5 h. Then 982.6 mg (9.9 mmol) trimethylsilylcyanide is added dropwise and stirred at RT for 48 h. The solvent is removed and the raw residue is used further.

LCMS (method 5): Rₜ=1.16 min (m/z=191 (M+H)⁺)

### Example 28A

### 3-(2-Amino-1-benzylethyl)-1,3-oxazolidin-2-one

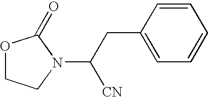

1583 mg (8.32 mmol) of 2-[(2-hydroxyethyl)amino]-3-phenylpropanenitrile and 2024 mg (12.49 mmol) 1,1′-carbonyl-bis(1H-imidazole) are dissolved together with 102 mg (0.832 mmol) DMAP in 30 ml acetonitrile and heated for 16 h at 60° C. The solvent is removed and the residue is purified by preparative HPLC. We obtain 123.8 mg (7% of th.) of product.

LCMS (method 6): Rₜ=3.08 min (m/z=217 (M+H)⁺)

¹H-NMR (400 MHz, DMSO-d₆): δ=7.25-7.37 (m, 5H), 5.13 (dd, 1H), 4.37 (sext, 1H), 4.25 (dd, 1H), 3.72 (sext, 1H), 3.51 (dd, 1H), 3.13-3.27 (m, 2H).

### Example 29A

### 3-(2-Amino-1-benzylethyl)-1,3-oxazolidin-2-one

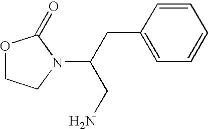

120 mg (0.56 mmol) of 3-(2-amino-1-benzylethyl)-1,3-oxazolidin-2-one (Example 28A) is dissolved in 5 ml methanol and, at 0° C., 264 mg (1.11 mmol) cobalt(II) chloride hexahydrate and then 214.1 mg (5.66 mmol) sodium boron hydride are added. It is stirred for 30 min at 0° C. and then allowed to reach RT. After approx. 1 h, 1N hydrochloric acid is added until the precipitate has dissolved and it is then made basic with conc. ammonia solution. The precipitate is filtered off. After removing the solvent we obtain 106.8 mg (84% of th.) of product.

LCMS (method 6): Rₜ=2.30 min (m/z=221 (M+H)⁺)

### Example 30A

### Morpholin-4-yl[2-(trifluoromethyl)phenyl]acetonitrile

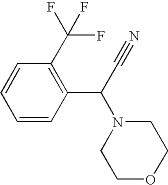

300 mg (1.72 mmol) 2-trifluorobenzaldehyde and 150.1 mg (1.72 mmol) morpholine are dissolved in 4 ml acetonitrile. Then 179.5 mg (1.81 mmol) trimethylsilyl cyanide is added and it is stirred for 16 h at RT. Saturated aqueous ammonium chloride solution is added to the preparation and it is extracted three times with ethyl acetate. After removing the solvent and purifying the residue by preparative HPLC we obtain 140.5 mg (30% of th.) of product.

LCMS (method 5): Rₜ=2.49 min (m/z=271 (M+H)⁺)

¹H-NMR (300 MHz, DMSO-d₆): δ=7.86 (d, 1H), 7.72-7.83 (m, 2H), 7.68 (t, 1H), 5.51 (s, 1H), 3.45-3.62 (m, 4H), 2.45-2.55 (m, 2H), 2.3-2.4 (m, 2H).

### Example 31A

### 2-Morpholin-4-yl-2-[2-(trifluoromethyl)phenyl]ethanamine

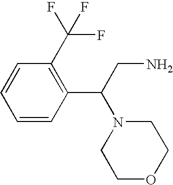

Similarly to the preparation procedure in Example 29A, we obtain from 140 mg (0.52 mmol) morpholin-4-yl[2-(trifluoromethyl)phenyl]acetonitrile after reduction with sodium boron hydride in the presence of cobalt(II) chloride hexahydrate, 76.1 mg (49% of th.) of product.

MS (ESIpos): m/z=275 (M+H)⁺.

¹H-NMR (300 MHz, DMSO-d₆): δ=7.80 (d, 1H), 7.70 (d, 1H), 7.67 (t, 1H), 7.47 (t, 1H), 3.48-3.62 (m, 4H), 2.85-2.98 (s, br, 3H), 2.40-2.55 (m, 2H), 2.21-2.31 (m, 2H).

### Example 32A

### 2,3-Dipyridin-2-ylpropanenitrile

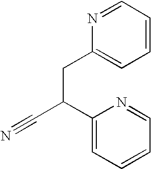

Similarly to the preparation procedure in Example 23A, we obtain from 750 mg (6.35 mmol) pyridin-2-ylacetonitrile, after reaction with 2-(bromomethyl)pyridine, 199 mg (15% of th.) of product.

LCMS (method 1): Rₜ=1.01 min (m/z=210 (M+H)⁺)

¹H-NMR (300 MHz, DMSO-d₆): δ=8.60 (d, 1H), 8.54 (d, 1H), 7.82 (dt, 1H), 7.74 (dt, 1H), 7.47 (d, 1H), 7.37 (dd, 1H), 7.32 (d, 1H), 7.27 (dd, 1H), 4.87 (dd, 1H), 3.38-3.47 (m, 2H).

### Example 33A

### 2,3-Dipyridin-2-ylpropan-1-amine

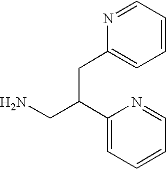

Similarly to the preparation procedure in Example 29A, we obtain from 199 mg (0.93 mmol) of 2,3-dipyridin-2-ylpropanenitrile, after reduction with sodium boron hydride in the presence of cobalt(II) chloride hexahydrate, 170 mg (86% of th.) of product.

MS (ESIpos): m/z=214 (M+H)⁺.

### Example 34A

### 4-(2-Oxopyrrolidin-1-yl)-2-pyridin-2-ylbutanenitrile

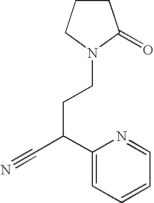

Similarly to the preparation procedure in Example 23A, we obtain from 750 mg (6.35 mmol) pyridin-2-ylacetonitrile, after reaction with 1-(2-chloroethyl)pyrrolidin-2-one, 1150 mg (40% of th.) of product.

LCMS (method 2): Rₜ=1.17 min (m/z=230 (M+H)⁺)

¹H-NMR (300 MHz, DMSO-d₆): δ=8.59 (d, 1H), 7.8-7.9 (m, 1H), 7.48 (d, 1H), 7.33-7.44 (m, 1H), 4.32 (dd, 1H), 3.72 (t, 2H), 3.50 (t, 2H), 3.39 (t, 2H), 2.23 (m, 2H), 1.82-1.97 (m, 2H).

### Example 35A

### 1-(4-Amino-3-pyridin-2-ylbutyl)pyrrolidin-2-one

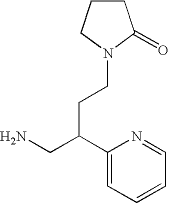

Similarly to the preparation procedure in Example 29A, we obtain from 1000 mg (2.18 mmol) of 2,3-4-(2-oxopyrrolidin-1-yl)-2-pyridin-2-ylbutanenitrile, after reduction with sodium boron hydride in the presence of cobalt(II) chloride hexahydrate, 360 mg (71% of th.) of product.

MS (ESIpos): m/z=234 (M+H)⁺.

¹H-NMR (300 MHz, DMSO-d₆): δ=8.57 (s, br, 1H), 7.77 (t, 1H), 7.3 (s, 1H), 6.53 (s, 2H), 6.31 (s, 1H), 3.72 (t, 2H), 3.50 (t, 2H), 3.39 (t, 2H), 3.0-3.25 (m, 3H), 2.28 (t, 2H), 1.7-2.0 (m, 2H).

### EXAMPLES OF APPLICATION

### Example 1

### N-(3-Chlorobenzyl)-2-[2-(4-methyl-1,3-thiazol-5-yl)ethyl]-2H-indazole-6-carboxamide

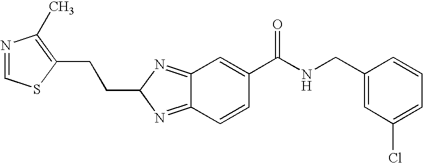

150 mg (0.48 mmol) of the indazole from Example 2A is put in 4 ml DMF and, successively, 233 mg (0.72 mmol) caesium carbonate, 116 mg (0.72 mmol) 5-(2-chloroethyl)-4-methyl-1,3-thiazole and a catalytic amount of potassium iodide are added. It is heated under argon for 16 h at 50° C. and the raw mixture, which contains two N-alkylated regioisomers in the approx. ratio 2.5:1, is purified by prep. HPLC. The desired compound is the isomer that formed in smaller amounts. We obtain 16 mg (8% of th.) of the indazole as resin.

MS (ESIpos): m/z=353 (M+H)⁺.

¹H-NMR (300 MHz, DMSO-d₆): δ=12.92 (s, 1H, broad), 10.66 (s, 1H), 8.28 (dd, 1H), 8.14 (dd, 1H), 7.98 (d, 2H), 7.73 (dd, 2H), 7.32 (m, 3H), 7.20 (t, 2H).

### Example 2

### N-(3-Chlorobenzyl)-2-(2-pyridin-2-ylethyl)-2H-indazole-6-carboxamide

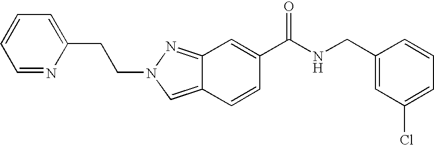

Similarly to the preparation procedure in Example 1, 150 mg (0.51 mmol) of the indazole from Example 2A is reacted with 109.3 mg (0.772 mmol) of 2-(2-chloroethyl)pyridine to the corresponding indazole derivative. We obtain, after purification by prep. HPLC, 6 mg (3% of th.) of product as resin.

MS (DCI(NH₃)): m/z=391 (M+H)⁺.

¹H-NMR (400 MHz, DMSO-d₆): δ=9.11 (t, 1H), 8.51 (d, 1H), 8.35 (s, 1H), 8.21 (s, 1H), 7.72 (d, 1H), 7.65 (dt, 1H), 7.50 (dd, 1H), 7.28-7.41 (m, 4H), 7.17-7.25 (m, 2H), 4.88 (t, 2H), 4.49 (d, 2H), 3.44 (t, 2H).

### Example 3

### N-(3-Chlorobenzyl)-2-[2-(1H-pyrazol-1-yl)ethyl]-2H-indazole-6-carboxamide

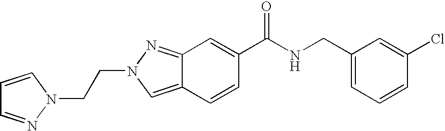

Similarly to the preparation procedure in Example 1, 500 mg (1.52 mmol) of the indazole from Example 2A is stirred with 399.7 mg (2.28 mmol) of 1-(2-bromoethyl)-1H-pyrazole at RT for 16 h and reacted to the corresponding indazole derivative. We obtain, after purification by prep. HPLC, 152.8 mg (25% of th.) of product as a solid.

LCMS (method 1): Rₜ=2.15 min (m/z=380 (M+H)⁺)

¹H-NMR (500 MHz, DMSO-d₆): δ=9.11 (t, 1H), 8.21 (s, 1H), 8.13 (s, 1H), 7.70 (d, 1H), 7.50 (d, 1H), 7.27-7.45 (m, 6H), 6.11 (s, 1H), 4.90 (t, 2H), 4.72 (t, 2H), 4.50 (d, 2H).

### Example 4

### N-(3-Chlorobenzyl)-2-[2-(2-oxo-1,3-oxazolidin-3-yl)ethyl]-2H-indazole-6-carboxamide

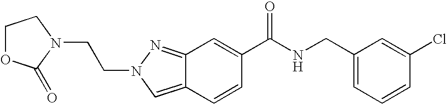

Similarly to the preparation procedure in Example 1, 250 mg (0.79 mmol) of the indazole from Example 2A is stirred with 179 mg (1.19 mmol) of 3-(2-chloroethyl)-1,3-oxazolidin-2-one at RT for 16 h and reacted to the corresponding indazole derivative. We obtain, after purification by prep. HPLC, 72.6 mg (22% of th.) of product as a solid.

MS (DCI(NH₃)): m/z=399 (M+H)⁺.

¹H-NMR (400 MHz, DMSO-d₆): δ=9.14 (t, 1H), 8.50 (s, 1H), 8.23 (s, 1H), 7.78 (d, 1H), 7.53 (dd, 1H), 7.28-7.41 (m, 4H), 4.65 (t, 2H), 4.50 (d, 2H), 4.16 (t, 2H), 3.73 (t, 2H), 3.37 (t, 2H).

### Example 5

### N-[5-Chloro-2-(1H-1,2,4-triazol-1-yl)benzyl]-2-[2-(1H-pyrazol-1-yl)ethyl]-2H-indazole-6-carboxamide

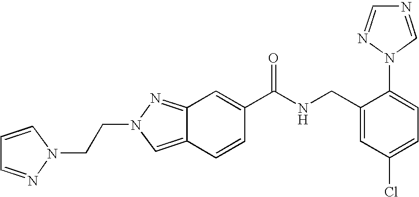

40 mg (0.156 mmol) of the carboxylic acid from Example 13A is put in DMF and 44.9 mg (0.23 mmol) EDC and 23.2 mg (0.17 mmol) HOBt are added. Then it is stirred for 2 h at RT. Then 76 mg (0.22 mmol) of the corresponding benzylamine (J. Med. Chem. 2004, 47, 2995) is added and the solution is stirred for 16 h at RT. We obtain, after purification by prep. HPLC, 37.7 mg (54% of th.) of product as a solid.

MS (DCI(NH₃)): m/z=447 (M+H)⁺.

¹H-NMR (400 MHz, DMSO-d₆): δ=9.01 (t, 1H), 8.99 (s, 1H), 8.29 (s, 1H), 8.14 (d, 2H), 7.70 (d, 1H), 7.55 (s, 3H), 7.43 (t, 3H), 6.11 (s, 1H), 4.90 (t, 2H), 4.72 (t, 2H), 4.41 (d, 2H).

### Example 6

### Methyl(2R)-2-(6-{[(3-chlorobenzyl)amino]carbonyl}-2H-indazol-2-yl)-3-pyridin-2-yl-propanoate

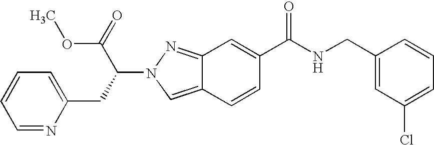

119.3 mg (0.47 mmol) of the bis-hydrochloride salt of methyl-3-pyridin-2-yl-L-alaninate is first transformed to the free base by treatment with Amberlyst A-21 in acetonitrile. This is dissolved in 2.5 ml trimethyl orthoformate and the aldehyde from Example 6A is added. It is stirred for 16 h at RT, then approx. 20 ml water is added and it is extracted three times with methyl-tert.-butyl ether. The combined ether phases are washed twice more with water and the organic phase is dried over magnesium sulphate. After removing the solvent, the imine that has formed is dissolved in 2 ml triethylphosphite and is heated for 3 h at 105° C. under argon. We obtain, after purification by prep. HPLC, 9 mg (5% of th.) of product as a solid.

LCMS (method 2): Rₜ=2.03 min (m/z=449 (M+H)⁺)

¹H-NMR (400 MHz, cDCl₃): δ=8.49 (d, 1H), 8.13 (s, 1H), 7.98 (s, 1H), 7.64 (d, 1H), 7.47 (d, 1H), 7.44 (dd, 1H), 7.35 (s, 1H), 7.28 (s, 1H), 7.08 (dd, 1H), 6.98 (d, 1H), 6.44 (t, 1H), 5.96 (dd, 1H), 4.65 (d, 2H), 3.78-3.93 (m, 2H), 3.75 (s, 3H), 1.3-1.38 (m, 2H).

### Example 7

### N-(3-Chlorobenzyl)-2-[2-(4-methoxyphenyl)-2-morpholin-4-ylethyl]-2H-indazole-6-carboxamide

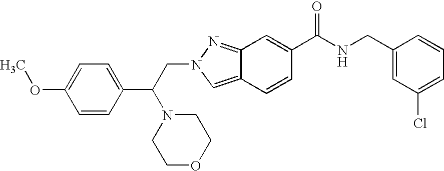

Similarly to the preparation procedure in Example 6, 70 mg (0.159 mmol) of the aldehyde from Example 6A is reacted with 48.7 mg (0.21 mmol) of 2-(4-methoxyphenyl)-2-morpholin-4-ylethanamine to the corresponding indazole derivative. We obtain, after purification by prep. HPLC, 23.6 mg (30% of th.) of product as a solid.

MS (DCI(NH₃)): m/z=505.6 (M+H)⁺.

¹H-NMR (400 MHz, DMSO-d₆): δ=9.10 (t, 1H), 8.25 (s, 1H), 8.16 (s, 1H), 7.70 (d, 1H), 7.46 (d, 1H), 7.25-7.40 (m, 4H), 7.19 (d, 2H), 6.84 (d, 2H), 5.76 (s, 1H), 5.03 (dd, 1H), 4.79 (dd, 1H), 4.48 (s, 2H), 4.19 (t, 1H), 3.70 (s, 3H), 3.49 (t, 4H), 2.45 (m, 1H), 2.21-2.35 (m, 2H).

### Example 8

### N-(3-Chlorobenzyl)-2-(2-phenylbutyl)-2H-indazole-6-carboxamide

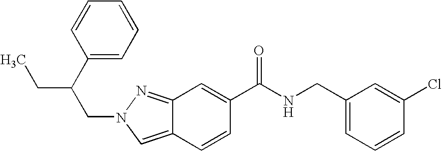

Similarly to the preparation procedure in Example 1, 150 mg (0.48 mmol) of the indazole from Example 2A is reacted with 203.6 mg (0.955 mmol) of [1-(bromo-methyl)propyl]benzene to the corresponding indazole derivative. We obtain, after purification and separation of the isomers by prep. HPLC, 11 mg (6% of th.) of product as a solid.

LCMS (method 2): Rₜ=2.69 min (m/z=418 (M+H)⁺)

¹H-NMR (400 MHz, cDCl₃): δ=8.14 (s, 1H), 7.59 (d, 1H), 7.54 (s, 1H), 7.46 (dd, 1H), 7.36 (s, 1H), 7.19-7.23 (m, 6H), 7.07 (d, 2H), 6.47 (t, 1H), 4.66 (d, 2H), 4.63 (dd, 1H), 4.49 (dd, 1H), 3.27 (pent, 1H), 1.65-1.78 (m, 2H), 0.83 (t, 3H).

### Example 9

### Ethyl-3-(6-{[(3-chlorobenzyl)amino]carbonyl}-2H-indazol-2-yl)-2-phenylpropanoate

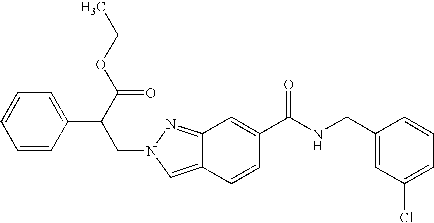

Similarly to the preparation procedure in Example 6, 527 mg (1.19 mmol) of the aldehyde from Example 6A is reacted with 300 mg (1.55 mmol) ethyl-3-amino-2-phenylpropanoate to the corresponding indazole derivative. We obtain, after purification by prep. HPLC, 125 mg (23% of th.) of product as a solid.

MS (DCI(NH₃)): m/z=462.5 (M+H)⁺.

¹H-NMR (400 MHz, DMSO-d₆): δ=9.13 (t, 1H), 8.34 (s, 1H), 8.19 (s, 1H), 7.72 (d, 1H), 7.50 (d, 1H), 7.21-7.40 (m, 9H), 5.11 (d, 1H), 4.83 (dd, 1H), 4.45-4.55 (m, 3H), 3.96-4.12 (m, 2H), 1.04 (t, 3H).

### Example 10

### N-(3-Chlorobenzyl)-2-[2-(3,5-dimethyl-1H-pyrazol-1-yl)ethyl]-2H-indazole-6-carboxamide

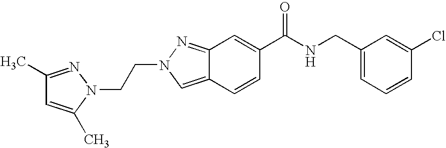

Similarly to the preparation procedure in Example 1, 150 mg (0.48 mmol) of the indazole from Example 2A is reacted with 113.7 mg (0.72 mmol) of 1-(2-chloroethyl)-3,5-dimethyl-1H-pyrazole to the corresponding indazole derivative. We obtain, after purification and separation of the isomers by prep. HPLC, 46 mg (23% of th.) of product as oil.

LCMS (method 1): Rₜ=2.24 min (m/z=408 (M+H)⁺)

¹H-NMR (400 MHz, DMSO-d₆): δ=9.12 (t, 1H), 8.22 (s, 1H), 8.10 (s, 1H), 7.71 (d, 1H), 7.51 (d, 1H), 7.28-7.41 (m, 4H), 5.66 (s, 1H), 4.81 (t, 2H), 4.49 (d, 2H), 4.47 (t, 2H), 2.08 (s, 3H), 1.71 (s, 3H).

### Example 11

### N-(3-Chlorobenzyl)-2-[2-(1H-1,2,3-triazol-1-yl)ethyl]-2H-indazole-6-carboxamide

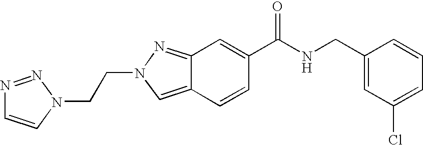

Similarly to the preparation procedure in Example 6, 40 mg (1.13 mmol) of the aldehyde from Example 6A is reacted with 14.1 mg (0.126 mmol) of 2-(1H-1,2,3-triazol-1-yl)ethanamine to the corresponding indazole derivative. We obtain, after purification by prep. HPLC, 5.2 mg (11% of th.) of product as oil.

LCMS (method 1): Rₜ=2.05 min (m/z=381 (M+H)⁺)

¹H-NMR (400 MHz, DMSO-d₆): δ=9.16 (t, 1H), 8.25 (s, 1H), 8.20 (s, 1H), 7.87 (s, 1H), 7.72 (d, 1H), 7.63 (s, 1H), 7.51 (dd, 1H), 7.27-7.41 (m, 4H), 4.96-5.06 (m, 4H), 4.49 (d, 2H).

### Example 12

### N-(3-Chlorobenzyl)-2-[2-(2-fluorophenyeethyl]-2H-indazole-6-carboxamide

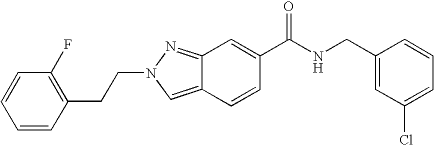

Similarly to the preparation procedure in Example 6, 50 mg (0.113 mmol) of the aldehyde from Example 6A is reacted with 20.49 mg (0.147 mmol) of 2-(2-fluorophenyl)ethanamine to the corresponding indazole derivative. We obtain, after purification by prep. HPLC, 27.1 mg (59% of th.) of product.

MS (DCI(NH₃)): m/z=408.5 (M+H)⁺.

¹H-NMR (400 MHz, DMSO-d₆): δ=9.12 (t, 1H), 8.34 (s, 1H), 8.21 (s, 1H), 7.72 (d, 1H), 7.50 (d, 1H), 7.35-7.41 (m, 3H), 7.29-7.33 (m, 1H), 7.25 (q, 1H), 7.15 (q, 2H), 7.05 (t, 1H), 4.72 (t, 2H), 4.50 (d, 2H), 3.32 (t, 2H).

### Example 13

### N-(3-Chlorobenzyl)-2-[2-(2-methyl-1,3-thiazol-4-yl)ethyl]-2H-indazole-6-carboxamide

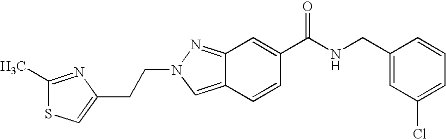

Similarly to the preparation procedure in Example 6, 50 mg (0.157 mmol) of the aldehyde from Example 6A is reacted with 28.03 mg (0.157 mmol) of the hydrochloride salt of 2-(2-methyl-1,3-thiazol-4-yl)ethanamine to the corresponding indazole derivative. We obtain, after purification by prep. HPLC, 3.95 mg (6% of th.) of product.

LCMS (method 1): Rₜ=2.35 min (m/z=411.7 (M+H)⁺)

¹H-NMR (400 MHz, DMSO-d₆): δ=9.11 (t, 1H), 8.38 (s, 1H), 8.22 (s, 1H), 7.73 (d, 1H), 7.51 (d, 1H), 7.28-7.42 (m, 4H), 7.09 (s, 1H), 4.79 (t, 2H), 4.50 (d, 2H), 3.35 (t, 2H), 2.63 (s, 3H).

### Example 14

### 2-{2-[5-(Aminocarbonyl)-1H-1,2,4-triazol-1-yl]ethyl}-N-(3-chlorobenzyl)-2H-indazole-6-carboxamide

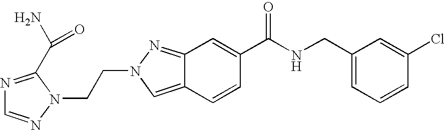

Similarly to the preparation procedure in Example 1, 150 mg (0.48 mmol) of the indazole from Example 2A is reacted with 166.8 mg (0.955 mmol) of 1-(2-chloroethyl)-1H-1,2,4-triazole-5-carboxamide to the corresponding indazole derivative. We obtain, after purification and separation of the isomers by prep. HPLC, 2.29 mg (1% of th.) of product as a solid.

LCMS (method 4): Rₜ=1.66 min (m/z=424.1 (M+H)⁺)

¹H-NMR (400 MHz, cDCl₃): δ=8.1 (s, 1H), 7.82 (s, 1H), 7.76 (s, 1H), 7.64 (d, 1H), 7.50 (dd, 1H), 7.36 (s, 1H), 7.21-7.31 (m, 3H), 7.06 (s, 1H, broad), 6.61 (t, 1H), 5.65 (s, 1H, broad), 5.27 (t, 2H), 4.94 (t, 2H), 4.65 (d, 2H).

### Example 15

### N-(3-Chlorobenzyl)-2-(2-phenylethyl)-2H-indazole-6-carboxamide

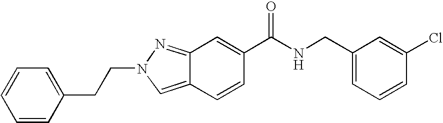

Similarly to the preparation procedure in Example 1, 250 mg (0.76 mmol) of the indazole from Example 2A is reacted with 211.31 mg (1.14 mmol) of (2-bromoethyl)benzene to the corresponding indazole derivative. We obtain, after purification and separation of the isomers by prep. HPLC, 89 mg (28% of th.) of product as a solid.

MS (DCI(NH₃)): m/z=390.4 (M+H)⁺.

¹H-NMR (400 MHz, DMSO-d₆): δ=9.12 (t, 1H), 8.32 (s, 1H), 8.22 (s, 1H), 7.72 (d, 1H), 7.50 (dd, 1H), 7.14-7.41 (m, 9H), 4.72 (t, 2H), 4.50 (d, 2H), 3.28 (t, 2H).

### Example 16

### N-(3-Chlorobenzyl)-2-(2-phenylpropyl)-2H-indazole-6-carboxamide

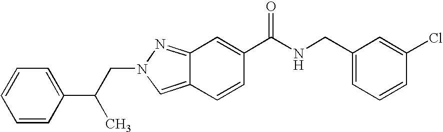

Similarly to the preparation procedure in Example 1, 150 mg (0.48 mmol) of the indazole from Example 2A is reacted with 142.7 mg (0.72 mmol) of (2-bromo-1-methylethyl)benzene to the corresponding indazole derivative. We obtain, after purification and separation of the isomers by prep. HPLC, 5.7 mg (3% of th.) of product as oil.

LCMS (method 1): Rₜ=2.66 min (m/z=404.2 (M+H)⁺)

¹H-NMR (400 MHz, cDCl₃): δ=8.15 (s, 1H), 7.62 (d, 1H), 7.61 (s, 1H), 7.48 (dd, 1H), 7.37 (s, 1H), 7.20-7.31 (m, 7H), 7.13 (d, 1H), 6.48 (t, 1H), 4.67 (d, 2H), 4.53 (d, 2H), 3.56 (tq, 1H), 1.31 (d, 3H).

### Example 17

### N-(3-Chlorobenzyl)-2-[2-(2,6-dichlorophenyl)ethyl]-2H-indazole-6-carboxamide

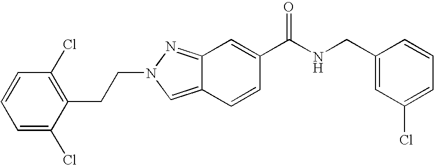

Similarly to the preparation procedure in Example 6, 70 mg (0.159 mmol) of the aldehyde from Example 6A is reacted with 39.18 mg (0.21 mmol) of 2-(2,6-dichlorophenyl)ethanamine to the corresponding indazole derivative. We obtain, after purification by prep. HPLC, 26 mg of product (33% of th.).

MS (DCI(NH₃)): m/z=458.4 (M+H)⁺.

¹H-NMR (400 MHz, DMSO-d₆): δ=9.12 (t, 1H), 8.38 (s, 1H), 8.22 (s, 1H), 7.74 (d, 1H), 7.51 (dd, 1H), 7.46 (d, 2H), 7.28-7.41 (m, 5H), 4.69 (t, 2H), 4.49 (d, 2H), 3.53 (t, 2H).

### Example 18

### N-(3-Chlorobenzyl)-2-[2-(4-chloro-1H-pyrazol-1-yl)ethyl]-2H-indazole-6-carboxamide

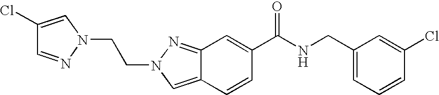

Similarly to the preparation procedure in Example 1, 150 mg (0.46 mmol) of the indazole from Example 2A is reacted with 113.1 mg (0.69 mmol) 4-chloro-1-(2-chloroethyl)-1H-pyrazole to the corresponding indazole derivative. We obtain, after purification and separation of the isomers by prep. HPLC, 24 mg (13% of th.) of product as oil.

LCMS (method 1): Rₜ=2.33 min (m/z=414 (M+H)⁺)

¹H-NMR (400 MHz, DMSO-d₆): δ=9.12 (t, 1H), 8.22 (s, 2H), 7.73 (d, 2H), 7.51 (t, 2H), 7.28-7.41 (m, 4H), 4.91 (t, 2H), 4.69 (t, 2H), 4.50 (d, 2H).

### Example 19

### 4-Chloro-N-(3-chlorobenzyl)-2-[2-(1H-pyrazol-1-yl)ethyl]-2H-indazole-6-carboxamide

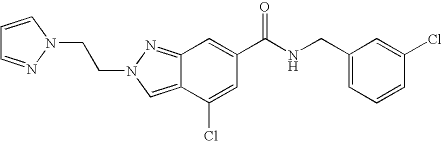

Similarly to the preparation procedure in Example 1, 80 mg (0.245 mmol) of the indazole from Example 11A is reacted with 63.9 mg (0.49 mmol) of 1-(2-chloroethyl)-1H-pyrazole to the corresponding 4-chloro-indazole derivative. We obtain, after purification and separation of the isomers by prep. HPLC, 25.8 mg (25% of th.) of product as crystals.

LCMS (method 2): Rₜ=2.28 min (m/z=414.2 (M+H)⁺)

¹H-NMR (400 MHz, DMSO-d₆): δ=9.22 (t, 1H), 8.30 (s, 1H), 8.21 (s, 1H), 7.58 (s, 1H), 7.43 (dd, 1H), 7.28-7.41 (m, 5H), 6.14 (t, 1H), 4.93 (t, 2H), 4.74 (t, 2H), 4.50 (d, 2H).

### Example 20

### 4-Chloro-N-(3-chlorobenzyl)-2-[2-(4-chloro-3,5-dimethyl-1H-pyrazol-1-yl)ethyl]-2H-indazole-6-carboxamide

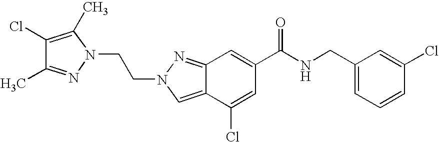

Similarly to the preparation procedure in Example 1, 80 mg (0.245 mmol) of the indazole from Example 11A is reacted with 94.55 mg (0.49 mmol) 4-chloro-1-(2-chloroethyl)-3,5-dimethyl-1H-pyrazole to the corresponding 4-chloro-indazole derivative. We obtain, after purification and separation of the isomers by prep. HPLC, 19.2 mg (16% of th.) of product as crystals.

LCMS (method 2): Rₜ=2.63 min (m/z=478.2 (M+H)⁺)

¹H-NMR (400 MHz, DMSO-d₆): δ=9.23 (t, 1H), 8.39 (s, 1H), 8.22 (s, 1H), 7.60 (s, 1H), 7.28-7.42 (m, 4H), 4.86 (t, 2H), 4.57 (t, 2H), 4.50 (d, 2H), 2.07 (s, 3H), 1.85 (s, 3H).

### Example 21

### 4-Chloro-N-(3-chlorobenzyl)-2-[2-(2-oxo-1,3-oxazolidin-3-yl)ethyl]-2H-indazole-6-carboxamide

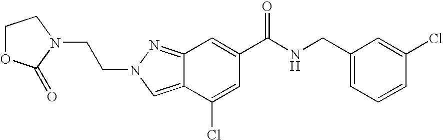

Similarly to the preparation procedure in Example 1, 80 mg (0.245 mmol) of the indazole from Example 11A is reacted with 149.58 mg (0.49 mmol) of 3-(2-chloroethyl)-1,3-oxazolidin-2-one to the corresponding 4-chloro-indazole derivative. We obtain, after purification and separation of the isomers by prep. HPLC, 23.3 mg (22% of th.) of product as crystals.

LCMS (method 2): Rₜ=2.11 min (m/z=433.2 (M+H)⁺)

¹H-NMR (400 MHz, DMSO-d₆): δ=9.23 (t, 1H), 8.68 (s, 1H), 8.23 (s, 1H), 7.60 (s, 1H), 7.28-7.41 (m, 4H), 4.68 (t, 2H), 4.50 (d, 2H), 4.18 (t, 2H), 3.75 (t, 2H), 3.43 (t, 2H).

### Example 22

### 4-Chloro-N-(3-chlorobenzyl)-2-(2-pyridin-2-ylethyl)-2H-indazole-6-carboxamide

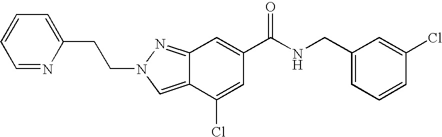

Similarly to the preparation procedure in Example 1, 80 mg (0.245 mmol) of the indazole from Example 11A is reacted with 141.6 mg (0.49 mmol) of 2-(2-chloroethyl)pyridine to the corresponding 4-chloro-indazole derivative. We obtain, after purification and separation of the isomers by prep. HPLC, 14.1 mg (13% of th.) of product as oil.

LCMS (method 2): Rₜ=2.05 min (m/z=425.2 (M+H)⁺)

¹H-NMR (400 MHz, DMSO-d₆): δ=9.21 (t, 1H), 8.54 (s, 1H), 8.51 (d, 1H), 8.21 (s, 1H), 7.67 (dt, 1H), 7.58 (s, 1H), 7.28-7.41 (m, 4H), 7.22 (d, 2H), 4.91 (t, 2H), 4.50 (d, 2H), 3.46 (t, 2H).

### Example 23

### N-(3-Chlorobenzyl)-4-methoxy-2-[2-(1H-pyrazol-1-yl)ethyl]-2H-indazole-6-carboxamide

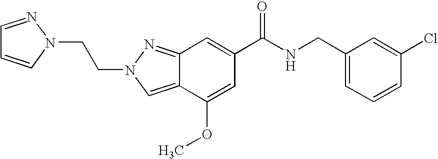

Similarly to the preparation procedure in Example 1, 30 mg (0.092 mmol) of the indazole from Example 18A is reacted with 32.3 mg (0.184 mmol) of 1-(2-bromoethyl)-1H-pyrazole to the corresponding 4-methoxy-indazole derivative. We obtain, after purification and separation of the isomers by prep. HPLC, 6 mg (14% of th.) of product as a solid.

LCMS (method 4): Rₜ=1.94 min (m/z=410 (M+H)⁺)

¹H-NMR (400 MHz, DMSO-d₆): δ=9.10 (t, 1H), 8.11 (s, 1H), 7.81 (s, 1H), 7.25-7.45 (m, 6H), 6.85 (s, 1H), 6.12 (t, 1H), 4.86 (t, 2H), 4.70 (t, 2H), 4.49 (d, 2H), 3.90 (s, 3H).

### Example 24

### N-(3-Chlorobenzyl)-2-[2-(4-chloro-3,5-dimethyl-1H-pyrazol-1-yl)ethyl]-4-methoxy-2H-indazole-6-carboxamide

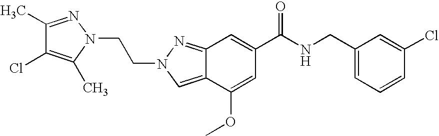

Similarly to the preparation procedure in Example 1, 30 mg (0.092 mmol) of the indazole from Example 18A is reacted with 35.6 mg (0.184 mmol) of 4-chloro-1-(2-chloroethyl)-3,5-dimethyl-1H-pyrazole to the corresponding 4-methoxy-indazole derivative. We obtain, after purification and separation of the isomers by prep. HPLC, 5 mg (11% of th.) of product as a solid.

LCMS (method 2): Rₜ=2.46 min (m/z=472 (M+H)+)

¹H-NMR (400 MHz, DMSO-d₆): δ=9.10 (t, 1H), 8.22 (s, 1H), 7.82 (s, 1H), 7.24-7.41 (m, 4H), 6.87 (s, 1H), 4.78 (t, 2H), 4.53 (t, 2H), 4.89 (d, 2H), 3.91 (s, 3H), 2.08 (s, 3H), 1.78 (s, 3H).

### Example 25

### N-(3-Chlorobenzyl)-4-cyano-2-[2-(2-oxo-1,3-oxazolidin-3-yl)ethyl]-2H-indazole-6-carboxamide

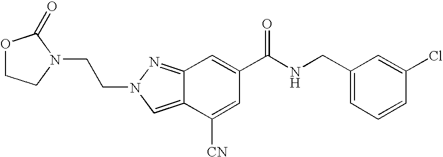

Similarly to the preparation procedure in Example 1, 80 mg (0.257 mmol) of the indazole from Example 22A is reacted with 77 mg (0.515 mmol) of 3-(2-chloroethyl)-1,3-oxazolidin-2-one to the corresponding indazole derivative. We obtain, after purification and separation of the isomers by prep. HPLC, 31 mg (27% of th.) of product as a solid.

MS (ESIpos): m/z=442 (M+H)⁺.

¹H-NMR (400 MHz, DMSO-d₆): δ=9.14 (t, 1H), 8.20 (d, 1H), 8.14 (s, 1H), 7.73 (d, 1H), 7.52 (d, 1H), 7.28-7.42 (m, 4H), 4.83 (t, 2H), 4.55 (t, 2H), 4.50 (d, 2H), 2.08 (s, 3H), 1.74 (s, 3H).

### Example 26

### N-(3-Chlorobenzyl)-2-[2-(4-chloro-3,5-dimethyl-1H-pyrazol-1-yl)ethyl]-2H-indazole-6-carboxamide

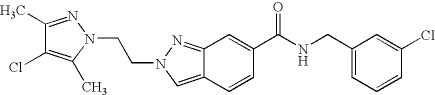

Similarly to the preparation procedure in Example 1, 150 mg (0.514 mmol) of the indazole from Example 2A is reacted with 149 mg (0.772 mmol) of 4-chloro-1-(2-chloroethyl)-3,5-dimethyl-1H-pyrazole to the corresponding indazole derivative. We obtain, after purification and separation of the isomers by prep. HPLC, 27 mg (12% of th.) of product as a solid.

MS (ESIpos): m/z=442 (M+H)⁺.

¹H-NMR (400 MHz, DMSO-d₆): δ=9.14 (t, 1H), 8.20 (d, 1H), 8.14 (s, 1H), 7.73 (d, 1H), 7.52 (d, 1H), 7.28-7.42 (m, 4H), 4.83 (t, 2H), 4.55 (t, 2H), 4.50 (d, 2H), 2.08 (s, 3H), 1.74 (s, 3H).

### Example 27

### 3-{6-[(3-Chlorobenzyl)carbamoyl]-2H-indazol-2-yl}-2-phenylpropionic acid

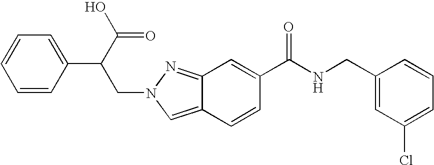

Similarly to the preparation procedure in Example 15A, 112 mg (0.242 mmol) of the ester from Example 9 is saponified to the corresponding acid. After purification by extraction, we obtain 84 mg (72% of th.) of product as a solid.

MS (ESIpos): m/z=434.1 (M+H)⁺.

¹H-NMR (400 MHz, DMSO-d₆): δ=12.74 (s, 1H), 9.12 (t, 1H), 8.33 (s, 1H), 8.19 (s, 1H), 7.71 (d, 1H), 7.49 (d, 1H), 7.22-7.41 (m, 9H), 5.08 (dd, 1H), 4.78 (dd, 1H), 4.49 (d, 2H), 4.44 (t, 1H).

### Example 28

### N-(3-Chlorobenzyl)-2-(3-morpholin-4-yl-3-oxo-2-phenylpropyl)-2H-indazole-6-carboxamide

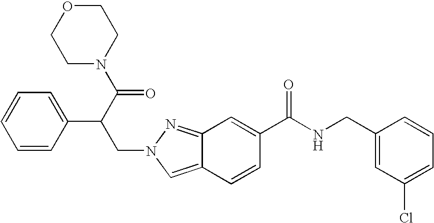

35 mg (0.081 mmol) of the acid from Example 27 is put in 2 ml dichloromethane and 1 ml DMF, 23.2 mg (0.121 mmol) EDC, 12 mg (0.09 mmol) HOBt, 20.9 mg (0.161 mmol) DIEA and then 9.8 mg (0.11 mmol) morpholine are added. The solution is stirred for 16 h at RT. 2M hydrochloric acid is added, and then separated by prep. HPLC. We obtain 15 mg (37% of th.) of the product as a solid.

MS (CI): m/z=503.3 (M)⁺.

¹H-NMR (400 MHz, DMSO-d₆): δ=9.12 (t, 1H), 8.25 (s, 1H), 8.19 (s, 1H), 7.71 (d, 1H), 7.50 (dd, 1H), 7.21-7.41 (m, 9H), 5.06 (dd, 1H), 4.88 (t, 1H), 4.67 (dd, 1H), 4.50 (d, 2H), 3.47-3.56 (m, 1H), 3.33-3.44 (m, 5H), 3.20-3.27 (m, 1H), 2.96-3.05 (m, 1H).

### Example 29

### N-(3-Chlorobenzyl)-2-(2,3-dipyridin-2-ylpropyl)-2H-indazole-6-carboxamide

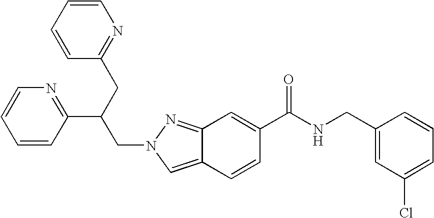

Similarly to the preparation procedure in Example 6, 70 mg (0.187 mmol) of the aldehyde from Example 6A is reacted with 159 mg (0.747 mmol) 2,3-dipyridin-2-ylpropan-1-amine (prepared as in Example 24A) to the corresponding indazole derivative. We obtain, after purification by prep. HPLC, 4 mg (4% of th.) of the product.

LCMS (method 5): Rₜ=1.83 min (m/z=482 (M+H)⁺)

¹H-NMR (400 MHz, DMSO-d₆): δ=9.09 (t, 1H), 8.53 (d, 1H), 8.44 (d, 1H), 8.18 (d, 1H), 7.66 (d, 1H), 7.55 (dt, 1H), 7.47 (t, 2H), 7.25-7.40 (m, 5H), 7.09-7.15 (m, 2H), 7.01 (d, 1H), 6.90 (d, 1H), 4.90 (dd, 1H), 4.78 (dd, 1H), 4.48 (d, 2H), 4.12-4.21 (m, 1H), 3.24 (dd, 1H), 3.05 (dd, 1H).

### Example 30

### N-(3-Chlorobenzyl)-2-(3-methyl-2-pyridin-2-ylpentyl)-2H-indazole-6-carboxamide

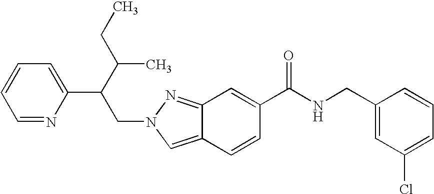

Similarly to the preparation procedure in Example 1, 137 mg (0.481 mmol) of the indazole from Example 2A is reacted with 190.2 mg (0.962 mmol) of 2-[-1-(chloromethyl)-2-methylbutyl]pyridine, which is obtained from the corresponding ethyl ester by reduction and subsequent transformation of the alcohol to the chloride under standard conditions, to the corresponding indazole derivative. We obtain, after purification and separation of the isomers by prep. HPLC, 11 mg (5% of th.) of product as oil.

MS (ESIpos): m/z=447 (M+H)⁺.

¹H-NMR (400 MHz, DMSO-d₆): δ=9.08 (t, 1H), 8.61 (s, 1H), 8.20 (d, 1H), 8.11 (d, 1H), 7.80-8.2 (m, broad, 1H), 7.65 (dd, 1H), 7.40-7.50 (m, 3H), 7.25-7.39 (m, 4H), 4.89-5.08 (m, 2H), 4.47 (d, 2H), 1.80-1.96 (m, 1H), 1.48-1.63 (m, 1H), 1.12-1.40 (m, 1H), 1.03 (m, 2H), 0.91 (t, 2H), 0.83 (t, 2H), 0.77 (d, 1H).

### Example 31

### N-(3-Chlorobenzyl)-2-[2-(2-oxopyridin-1(2H)-yl)ethyl]-2H-indazole-6-carboxamide

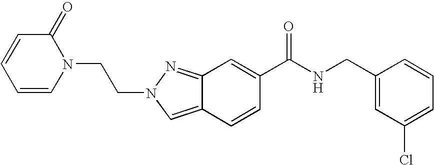

Similarly to the preparation procedure in Example 1, 150 mg (0.53 mmol) of the indazole from Example 2A is reacted with 157.6 mg (0.79 mmol) of 1-(2-chloroethyl)pyridin-2(1H)-one to the corresponding indazole derivative. We obtain, after purification and separation of the isomers by prep. HPLC, 37 mg (17% of th.) of product as a solid.

MS (ESIpos): m/z=447 (M+H)⁺.

¹H-NMR (400 MHz, DMSO-d₆): δ=9.13 (t, 1H), 8.34 (s, 1H), 8.21 (s, 1H), 7.73 (d, 1H), 7.52 (d, 1H), 7.27-7.41 (m, 5H), 7.03 (d, 1H), 6.38 (d, 1H), 5.95 (t, 1H), 4.80 (t, 2H), 4.50 (d, 2H), 4.45 (t, 2H).

### Example 32

### N-(3-Chlorobenzyl)-2-[2-pyridin-2-yl-3-(tetrahydro-2H-pyran-4-yl)propyl]-2H-indazole-6-carboxamide

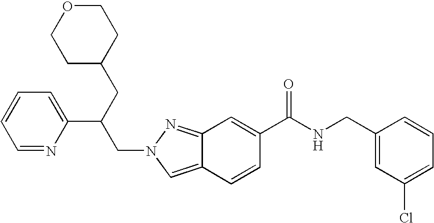

Similarly to the preparation procedure in Example 6, 70 mg (0.187 mmol) of the aldehyde from Example 6A is reacted with 53.47 mg (0.24 mmol) 2-pyridin-2-yl-3-(tetrahydro-2H-pyran-4-yl)propan-1-amine (Example 24A) to the corresponding indazole derivative. We obtain, after purification by prep. HPLC, 14 mg (12% of th.) of product as oil.

LCMS (method 2): Rₜ=2.11 min (m/z=489 (M+H)⁺)

¹H-NMR (400 MHz, DMSO-d₆): δ=9.09 (t, 1H), 8.54 (d, 1H), 8.15 (d, 2H), 7.66 (d, 1H), 7.59 (dt, 1H), 7.46 (d, 1H), 7.27-7.40 (m, 4H), 7.19 (dd, 1H), 7.14 (d, 1H), 4.71 (m, 2H), 4.48 (d, 2H), 3.63-3.79 (m, 3H), 3.40-3.48 (m, 1H), 3.08 (dd, 2H), 1.82 (m, 1H), 1.50-1.61 (m, 1H), 1.28-1.47 (m, 2H), 0.98-1.22 (m, 2H).

### Example 33

### N-(3-Chlorobenzyl)-2-[2-(4-chloro-3,5-dimethyl-1H-pyrazol-1-yl)ethyl]-4-cyano-2H-indazole-6-carboxamide

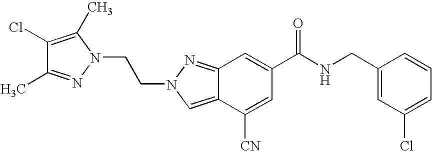

Similarly to the preparation procedure in Example 1, 80 mg (0.26 mmol) of the indazole from Example 22A is reacted with 99.4 mg (0.52 mmol) 4-chloro-1-(2-chloroethyl)-3,5-dimethyl-1H-pyrazole to the corresponding indazole derivative. We obtain, after purification and separation of the isomers by prep. HPLC, 23 mg (19% of th.) of product as a solid.

MS (DCI, NH₃): m/z=467 (M+H)⁺.

¹H-NMR (400 MHz, DMSO-d₆): δ=9.31 (t, 1H), 8.63 (s, 1H), 8.58 (s, 1H), 8.15 (s, 1H), 7.29-7.44 (m, 4H), 4.90 (t, 2H), 4.59 (t, 2H), 4.52 (d, 2H), 2.05 (s, 3H), 1.88 (s, 3H).

### Example 34

### N-(3-Chlorobenzyl)-2-[2-(3,5-dimethyl-1H-pyrazol-1-yl)propyl]-2H-indazole-6-carboxamide

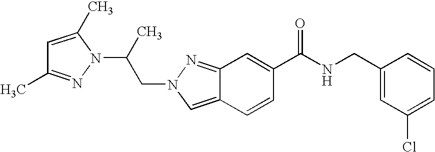

Similarly to the preparation procedure in Example 6, 50 mg (0.157 mmol) of the aldehyde from Example 6A is reacted with 35.5 mg (0.157 mmol) of 2-(3,5-dimethyl-1H-pyrazol-1-yl)propan-1-amine dihydrochloride to the corresponding indazole derivative. We obtain, after purification by prep. HPLC, 11 mg (16% of th.) of product.

MS (DCI, NH₃): m/z=422.5 (M+H)⁺.

¹H-NMR (400 MHz, DMSO-d₆): δ=9.13 (t, 1H), 8.20 (s, 1H), 7.92 (s, 1H), 7.68 (d, 1H), 7.49 (dd, 1H), 7.28-7.41 (m, 5H), 4.83 (m, 1H), 4.75 (d, 2H), 4.49 (d, 2H), 2.12 (s, 3H), 1.72 (s, 3H), 1.43 (d, 3H).

### Example 35

### N-(3-Chlorobenzyl)-2-[2-pyridin-2-yl-3-(tetrahydro-2H-pyran-2-yl)propyl]-2H-indazole-6-carboxamide

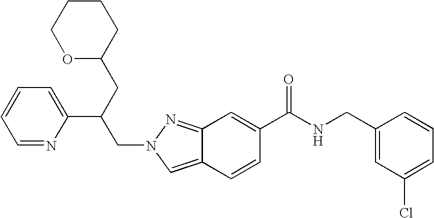

Similarly to the preparation procedure in Example 6, 70 mg (0.158 mmol) of the aldehyde from Example 6A is reacted with 139.4 mg (0.633 mmol) 2-pyridin-2-yl-3-(tetrahydro-2H-pyran-2-yl)propan-1-amine (prepared as in Example 24A) to the corresponding indazole derivative. We obtain, after purification by prep. HPLC, 14 mg (12% of th.) of diastereomerically pure product as oil.

LCMS (method 2): Rₜ=2.11 min (m/z=489 (M+H)⁺)

¹H-NMR (400 MHz, DMSO-d₆): δ=9.09 (t, 1H), 8.54 (d, 1H), 8.15 (d, 2H), 7.66 (d, 1H), 7.59 (dt, 1H), 7.46 (d, 1H), 7.27-7.40 (m, 4H), 7.19 (dd, 1H), 7.14 (d, 1H), 4.71 (m, 2H), 4.48 (d, 2H), 3.63-3.79 (m, 3H), 3.40-3.48 (m, 1H), 3.08 (dd, 2H), 1.82 (m, 1H), 1.50-1.61 (m, 1H), 1.28-1.47 (m, 2H), 0.98-1.22 (m, 2H).

### Example 36

### N-[5-Chloro-2-(1H-1,2,4-triazol-1-yl)benzyl]-2-(2-ethylbutyl)-2H-indazole-6-carboxamide

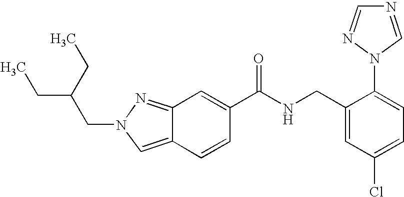

13.6 mg (0.055 mmol) of the acid from Example 26A is put in 3 ml dichloromethane and 14.9 mg (0.072 mmol) 1-[5-chloro-2-(1H-1,2,4-triazol-1-yl)phenyl]methanamine (J. Med. Chem. 2004, 47, 2995-3008), 10.08 mg (0.083 mmol) DMAP and 21.09 mg (0.11 mmol) EDC are added. The suspension is stirred for 24 h at RT. Citric acid solution is added and it is extracted three times with ethyl acetate. The combined organic phases are washed with saturated aqueous sodium chloride solution and then dried over magnesium sulphate. After removal of the solvent and separation by prep. HPLC we obtain 3 mg (12% of th.) of the product as oil.

LCMS (method 1): Rₜ=2.20 min (m/z=437 (M+H)⁺)

¹H-NMR (400 MHz, cDCl₃): δ=8.49 (s, 1H), 8.30 (s, 1H), 8.18 (s, 1H), 7.93 (s, 1H), 7.80 (d, 1H), 7.71 (d, 1H), 7.59 (m, 2H), 7.41 (dd, 1H), 7.28 (d, 1H), 4.51 (d, 2H), 4.34 (d, 2H), 2.66 (s, broad, 1H), 2.05 (pent, 1H), 1.23-1.40 (m, 4H), 0.91 (t, 6H).

### Example 37

### N-(3-Chlorobenzyl)-2-[2-(2-oxo-1,3-oxazolidin-3-yl)pentyl]-2H-indazole-6-carboxamide

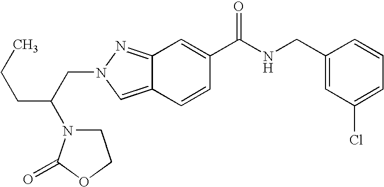

Similarly to the preparation procedure in Example 6, 70 mg (0.159 mmol) of the aldehyde from Example 6A is reacted with 45.22 mg (0.21 mmol) 3-[1-(aminomethyl)butyl]-1,3-oxazolidin-2-one to the corresponding indazole derivative. We obtain, after purification by prep. HPLC, 35 mg (50% of th.) of product.

MS (DCI, NH₃): m/z=457.8 (M+NH₄)⁺.

¹H-NMR (400 MHz, DMSO-d₆): δ=9.14 (t, 1H), 8.46 (s, 1H), 8.21 (s, 1H), 7.77 (d, 1H), 7.52 (d, 1H), 7.28-7.41 (m, 4H), 4.55-4.65 (m, 1H), 4.50 (d, 2H), 4.12-4.28 (m, 3H), 3.71 (dd, 1H), 3.37 (q, 1H), 1.56-1.68 (m, 1H), 1.44-1.55 (m, 1H), 1.18-1.40 (m, 3H), 0.90 (t, 3H).

### Example 38

### N-(3-Chlorobenzyl)-2-[2-(2-chlorophenyl)ethyl]-2H-indazole-6-carboxamide

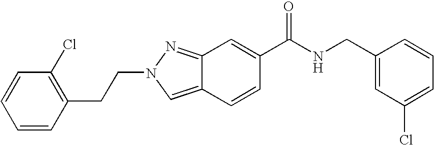

Similarly to the preparation procedure in Example 6, 50 mg (0.157 mmol) of the aldehyde from Example 6A is reacted with 24.4 mg (0.157 mmol) of 2-(2-chlorophenyl)ethylamine to the corresponding indazole derivative. We obtain, after purification by prep. HPLC, 13 mg (20% of th.) of product.

MS (DCI, NH₃): m/z=424.4 (M+H)⁺.

¹H-NMR (400 MHz, DMSO-d₆): δ=9.12 (t, 1H), 8.35 (s, 1H), 8.22 (s, 1H), 7.23 (d, 1H), 7.51 (dd, 1H), 7.44 (d, 1H), 7.15-7.41 (m, 7H), 4.73 (t, 2H), 4.50 (d, 2H), 3.40 (t, 2H).

### Example 39

### N-(3-Chlorobenzyl)-2-[2-(4-methoxyphenyl)ethyl]-2H-indazole-6-carboxamide

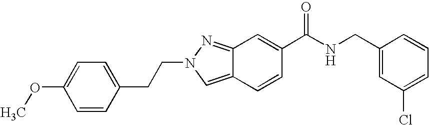

Similarly to the preparation procedure in Example 6, 50 mg (0.157 mmol) of the aldehyde from Example 6A is reacted with 24.4 mg (0.157 mmol) of 2-(4-methoxyphenyl)ethanamine to the corresponding indazole derivative. We obtain, after purification by prep. HPLC, 11 mg (17% of th.) of product.

MS (DCI, NH₃): m/z=420.4 (M+H)⁺.

¹H-NMR (400 MHz, DMSO-d₆): δ=9.12 (t, 1H), 8.31 (s, 1H), 8.22 (s, 1H), 7.72 (d, 1H), 7.50 (d, 1H), 7.28-7.41 (m, 4H), 7.06 (d, 2H), 6.79 (d, 2H), 4.67 (t, 2H), 4.50 (d, 2H), 3.39 (s, 3H), 3.21 (t, 2H).

### Example 40

### N-(3-Chlorobenzyl)-2-[2-(2-oxo-1,3-oxazolidin-3-yl)-3-phenylpropyl]-2H-indazole-6-carboxamide

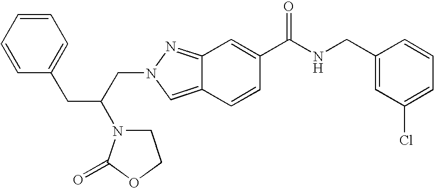

Similarly to the preparation procedure in Example 6, 70 mg (0.159 mmol) of the aldehyde from Example 6A is reacted with 57.84 mg (0.206 mmol) of 3-(2-amino-1-benzylethyl)-1,3-oxazolidin-2-one to the corresponding indazole derivative. We obtain, after purification by prep. HPLC, 41 mg (50% of th.) of product.

MS (DCI, NH₃): m/z=505.8 (M+NH₄)⁺.

¹H-NMR (400 MHz, DMSO-d₆): δ=9.13 (t, 1H), 8.49 (s, 1H), 8.21 (s, 1H), 7.77 (d, 1H), 7.52 (dd, 1H), 7.19-7.41 (m, 8H), 4.65-4.75 (m, 2H), 4.46-4.56 (m, 4H), 4.02-4.15 (m, 2H), 3.62-3.70 (m, 1H), 3.36-3.46 (m, 1H), 2.89-3.03 (m, 2H).

### Example 41

### N-[(5-Chloro-2-thienyemethyl]-2-[2-(1H-pyrazol-1-yl)ethyl]-2H-indazole-6-carboxamide

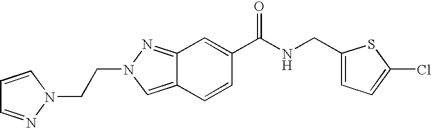

Similarly to the preparation procedure in Example 5, 50 mg (0.195 mmol) of the carboxylic acid from Example 13A is reacted with 40.3 mg (0.273 mmol) of 1-(5-chloro-2-thienyl)methanamine to the corresponding amide. We obtain, after purification by prep. HPLC, 46 mg (60% of th.) of product.

MS (DCI, NH₃): m/z=386 (M+H)⁺.

¹H-NMR (400 MHz, DMSO-d₆): δ=9.22 (t, 1H), 8.14 (d, 2H), 7.70 (d, 1H), 7.47 (d, 1H), 7.40 (d, 2H), 6.96 (d, 1H), 6.90 (d, 1H), 6.11 (s, 1H), 4.90 (t, 2H), 4.71 (t, 2H), 4.55 (d, 2H).

### Example 42

### N-(3-Chlorobenzyl)-2-[4-(2-oxopyrrolidin-1-yl)-2-pyridin-2-ylbutyl]-2H-indazole-6-carboxamide

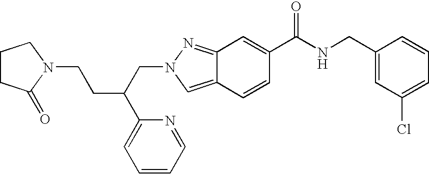

Similarly to the preparation procedure in Example 6, 70 mg (0.187 mmol) of the aldehyde from Example 6A is reacted with 174.2 mg (0.75 mmol) of 1-(4-amino-3-pyridin-2-ylbutyl)pyrrolidin-2-one (synthesis as in Example 24A) to the corresponding indazole derivative. We obtain, after purification by prep. HPLC, 4 mg (4% of th.) of product.

LCMS (method 2): Rₜ=1.88 min (m/z=502 (M+H)⁺)

### Example 43

### N-(3-Chlorobenzyl)-2-[4-methyl-2-(2-oxo-1,3-oxazolidin-3-yl)pentyl]-2H-indazole-6-carboxamide

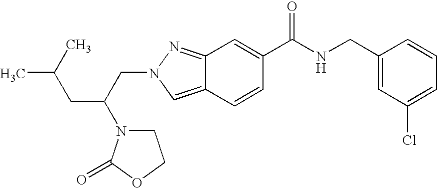

Similarly to the preparation procedure in Example 6, 70 mg (0.159 mmol) of the aldehyde from Example 6A is reacted with 48.9 mg (0.206 mmol) 3-[1-(aminomethyl)-3-methylbutyl]-1,3-oxazolidin-2-one to the corresponding indazole derivative. We obtain, after purification by prep. HPLC, 15 mg (20% of th.) of product.

LCMS (method 4): Rₜ=2.13 min (m/z=455 (M+H)⁺)

¹H-NMR (400 MHz, DMSO-d₆): δ=9.14 (t, 1H), 8.47 (s, 1H), 8.21 (s, 1H), 7.76 (d, 1H), 7.52 (dd, 1H), 7.27-7.42 (m, 4H), 4.54-4.63 (m, 1H), 4.46-4.54 (m, 2H), 4.14-4.35 (m, 3H), 3.67-3.78 (m, 1H), 3.78 (q, 1H), 1.45-1.68 (m, 2H), 1.15-1.34 (m, 2H), 0.92 (d, 3H), 0.88 (d, 3H).

### Example 44

### N-(3-Chlorobenzyl)-2-(3-cyclohexyl-2-pyridin-2-ylpropyl)-2H-indazole-6-carboxamide

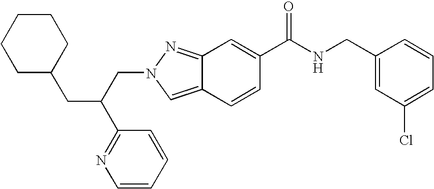

Similarly to the preparation procedure in Example 6, 70 mg (0.159 mmol) of the aldehyde from Example 6A is reacted with 57.33 mg (0.206 mmol) 3-cyclohexyl-2-pyridin-2-ylpropan-1-amine to the corresponding indazole derivative. We obtain, after purification by prep. HPLC, 20 mg (21% of th.) of product.

LCMS (method 4): Rₜ=2.58 min (m/z=487 (M+H)⁺)

¹H-NMR (400 MHz, DMSO-d₆): δ=9.10 (t, 1H), 8.56 (d, 1H), 8.17 (d, 2H), 7.66 (d, 2H), 7.46 (dd, 1H), 7.20-7.40 (m, 6H), 4.64-4.78 (m, 2H), 4.48 (d, 2H), 3.65-3.75 (m, 1H), 1.70-1.82 (m, 2H), 1.33-1.62 (m, 5H), 0.70-1.30 (m, 6H).

### Example 45

### N-(3-Chlorobenzyl)-2-[2-(2-chlorophenyl)-2-(diethylamino)ethyl]-2H-indazole-6-carboxamide

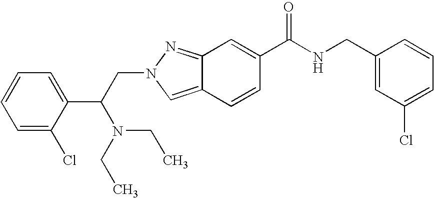

Similarly to the preparation procedure in Example 6, 60.79 mg (0.138 mmol) of the aldehyde from Example 6A is reacted with 40.59 mg (0.179 mmol) of 1-(2-chlorophenyl)-N¹,N¹-diethylethane-1,2-diamine to the corresponding indazole derivative. We obtain, after purification by prep. HPLC, 27 mg (40% of th.) of product.

LCMS (method 4): Rₜ=1.66 min (m/z=495 (M+H)⁺)

¹H-NMR (400 MHz, DMSO-d₆): δ=9.08 (t, 1H), 8.25 (s, 1H), 8.19 (s, 1H), 7.67-7.74 (m, 2H), 7.46 (dd, 1H), 7.22-7.40 (m, 7H), 4.97-5.09 (m, 2H), 4.77-4.85 (dd, 1H), 4.48 (d, 2H), 2.60-2.75 (m, 2H), 2.40-2.50 (m, 2H), 0.87 (t, 6H).

### Example 46

### N-(3-Chlorobenzyl)-2-[2-(4-methoxyphenyl)-2-piperidin-1-ylethyl]-2H-indazole-6-carboxamide

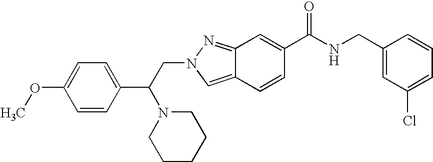

Similarly to the preparation procedure in Example 6, 60.79 mg (0.138 mmol) of the aldehyde from Example 6A is reacted with 41.95 mg (0.179 mmol) of 2-(4-methoxyphenyl)-2-piperidin-1-ylethanamine to the corresponding indazole derivative. We obtain, after purification by prep. HPLC, 32 mg (45% of th.) of product.

LCMS (method 4): Rₜ=1.59 min (m/z=503 (M+H)⁺)

¹H-NMR (400 MHz, DMSO-d₆): δ=9.08 (t, 1H), 8.27 (s, 1H), 8.16 (s, 1H), 7.70 (d, 1H), 7.46 (dd, 1H), 7.26-7.40 (m, 4H), 4.20 (d, 2H), 6.84 (d, 2H), 4.94-5.04 (m, 1H), 4.76-4.86 (m, 1H), 4.89 (d, 2H), 4.22 (t, 1H), 3.71 (s, 3H), 2.42-2.48 (m, 2H), 2.14-2.26 (m, 2H), 1.41 (m, 4H), 1.20-1.30 (m, 2H).

### Example 47

### N-(3-Chlorobenzyl)-2-[2-(4-methylpiperazin-1-yl)-2-phenylethyl]-2H-indazole-6-carboxamide

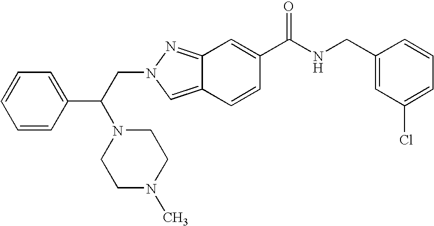

Similarly to the preparation procedure in Example 6, 60.79 mg (0.138 mmol) of the aldehyde from Example 6A is reacted with 39.21 mg (0.179 mmol) of 2-(4-methylpiperazin-1-yl)-2-phenylethanamine to the corresponding indazole derivative. We obtain, after purification by prep. HPLC, 18 mg (26% of th.) of product.

LCMS (method 4): Rₜ=1.53 min (m/z=488 (M+H)⁺)

¹H-NMR (400 MHz, DMSO-d₆): δ=9.08 (t, 1H), 8.26 (s, 1H), 8.17 (s, 1H), 7.70 (d, 1H), 7.47 (dd, 1H), 7.21-7.41 (m, 10H), 5.06 (dd, 1H), 4.82 (dd, 1H), 4.49 (d, 2H), 4.31 (m, 1H), 2.51-2.53 (m, 2H), 2.49 (s, 3H), 2.0-2.50 (m, 5H).

### Example 48

### N-(3-Chlorobenzyl)-2-(2-morpholin-4-yl-2-phenylethyl)-2H-indazole-6-carboxamide

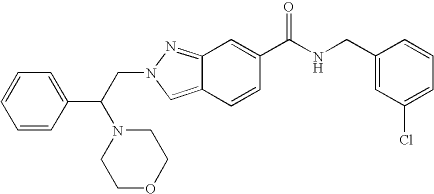

Similarly to the preparation procedure in Example 6, 60.79 mg (0.138 mmol) of the aldehyde from Example 6A is reacted with 39.26 mg (0.179 mmol) 2-morpholin-4-yl-2-phenylethanamine to the corresponding indazole derivative. We obtain, after purification by prep. HPLC, 18 mg (26% of th.) of product.

LCMS (method 4): Rₜ=1.83 min (m/z=475 (M+H)⁺)

¹H-NMR (400 MHz, DMSO-d₆): δ=9.08 (t, 1H), 8.25 (s, 1H), 8.16 (s, 1H), 7.69 (d, 1H), 7.46 (d, 1H), 7.21-7.40 (m, 9H), 5.07 (dd, 1H), 4.82 (dd, 1H), 4.48 (d, 2H), 4.24 (t, 1H), 2.47 (m, 2H), 2.27-2.37 (m, 2H), 2.17 (t, 2H), 1.90 (m, 2H).

### Example 49

### N-(3-Chlorobenzyl)-2-[2-(2-chlorophenyl)-2-(dimethylamino)ethyl]-2H-indazole-6-carboxamide

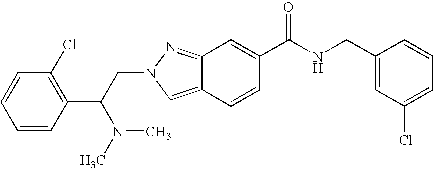

228.5 mg (1.15 mmol) of 1-(2-chlorophenyl)-N¹,N¹-dimethylethane-1,2-diamine is dissolved in 6.5 ml methanol, some molecular sieve (4A) is added and then 300 mg (0.885 mmol) of the aldehyde from Example 6A is added. It is stirred for 15 h at RT and then, after filtering off the molecular sieve, all volatile constituents are removed under vacuum. The imine formed is dissolved in 1.5 ml triethylphosphite and heated for 3 h at 105° C. under argon. We obtain, after purification by preparative HPLC, 158 mg (37% of th.) of product as a solid.

LCMS (method 7): Rₜ=2.39 min (m/z=467 (M+H)⁺)

¹H-NMR (400 MHz, DMSO-d₆): δ=9.09 (t, 1H), 8.25 (s, 1H), 8.18 (s, 1H), 7.69 (d, 1H), 7.59 (d, 1H), 7.21-7.41 (m, 8H), 5.05 (dd, 1H), 4.83 (m, 2H), 4.48 (d, 2H), 2.20 (s, 6H).

### Example 50

### N-(3-Chlorobenzyl)-2-[2-(2-chlorophenyl)-2-pyrrolidin-1-ylethyl]-2H-indazole-6-carboxamide

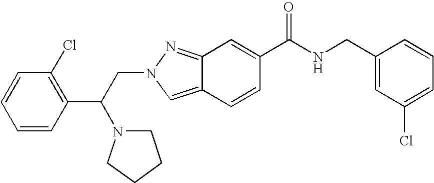

Similarly to the preparation procedure in Example 6, 60.79 mg (0.138 mmol) of the aldehyde from Example 6A is reacted with 40.23 mg (0.179 mmol) of 2-(2-chlorophenyl)-2-pyrrolidin-1-ylethanamine to the corresponding indazole derivative. We obtain, after purification by preparative HPLC, 29.7 mg (44% of th.) of product.

LCMS (method 1): Rₜ=1.59 min (m/z=493 (M+H)⁺)

¹H-NMR (400 MHz, DMSO-d₆): δ=9.09 (t, 1H), 8.15 (s, 1H), 8.09 (s, 1H), 7.66 (d, 1H), 7.54 (d, 1H), 7.45 (d, 1H), 7.34-7.40 (m, 2H), 7.25-7.33 (m, 4H), 7.19-7.24 (m, 1H), 5.0 (dd, 1H), 4.80 (dd, 1H), 4.65 (t, 1H), 4.48 (d, 2H), 2.4-2.62 (m, 4H), 1.61-1.70 (m, 4H).

### Example 51

### N-(3-Chlorobenzyl)-2-[2-(4-fluorophenyl)-2-morpholin-4-ylethyl]-2H-indazole-6-carboxamide

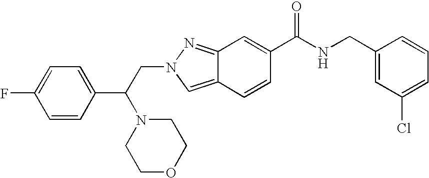

Similarly to the preparation procedure in Example 6, 60.79 mg (0.138 mmol) of the aldehyde from Example 6A is reacted with 40.15 mg (0.179 mmol) of 2-(4-fluorophenyl)-2-morpholin-4-ylethanamine to the corresponding indazole derivative. We obtain, after purification by preparative HPLC, 24.3 mg (35% of th.) of product.

LCMS (method 1): Rₜ=1.96 min (m/z=493 (M+H)⁺)

¹H-NMR (400 MHz, DMSO-d₆): δ=9.09 (t, 1H), 8.25 (s, 1H), 8.16 (s, 1H), 7.70 (d, 1H), 7.47 (d, 1H), 7.27-7.40 (m, 6H), 7.11 (t, 2H), 5.06 (dd, 1H), 4.82 (dd, 1H), 4.49 (d, 2H), 4.26 (t, 1H), 3.51 (t, 4H), 2.40-2.50 (m, 2H), 2.27-2.37 (m, 2H).

### Example 52

### N-(3-Chlorobenzyl)-2-[2-(2-fluorophenyl)-2-pyrrolidin-1-ylethyl]-2H-indazole-6-carboxamide

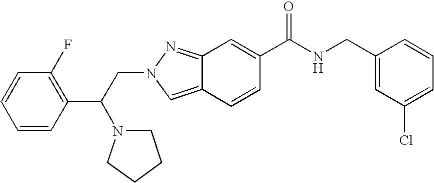

Similarly to the preparation procedure in Example 6, 60.79 mg (0.138 mmol) of the aldehyde from Example 6A is reacted with 37.3 mg (0.179 mmol) of 2-(2-fluorophenyl)-2-pyrrolidin-1-ylethanamine to the corresponding indazole derivative. We obtain, after purification by preparative HPLC, 29.7 mg (44% of th.) of product.

LCMS (method 8): Rₜ=1.56 min (m/z=477 (M+H)⁺)

¹H-NMR (400 MHz, DMSO-d₆): δ=9.09 (t, 1H), 8.19 (s, 1H), 8.15 (s, 1H), 7.66 (d, 1H), 7.42-7.51 (m, 2H), 7.21-7.39 (m, 5H), 7.14 (t, 1H), 7.04 (t, 1H), 5.03 (dd, 1H), 4.83 (dd, 1H), 4.54 (t, 1H), 4.48 (d, 2H), 2.4-2.55 (m, 4H), 1.60-1.67 (m, 4H).

### Example 53

### N-(3-Chlorobenzyl)-2-{2-morpholin-4-yl-2-[2-(trifluoromethyl)phenyl]ethyl}-2H-indazole-6-carboxamide

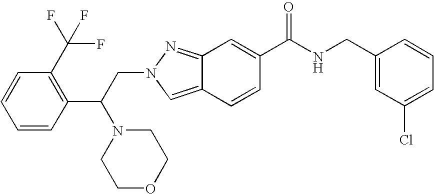

Similarly to the preparation procedure in Example 6, 58.48 mg (0.172 mmol) of the aldehyde from Example 6A is reacted with 75 mg (0.224 mmol) of 2-morpholin-4-yl-2-[2-(trifluoromethyl)-phenyl]ethanamine to the corresponding indazole derivative. We obtain, after purification by preparative HPLC, 30 mg (29% of th.) of product as a solid.

LCMS (method 5): Rₜ=2.52 min (m/z=543 (M+H)⁺)

¹H-NMR (400 MHz, DMSO-d₆): δ=9.07 (t, 1H), 8.16 (s, 1H), 8.09 (s, 1H), 7.75 (d, 1H), 7.67 (d, 2H), 7.64 (d, 1H), 7.48 (t, 2H), 7.27-7.40 (m, 4H), 5.08 (dd, 1H), 4.83 (dd, 1H), 4.41-4-52 (m, 3H), 3.51 (t, 4H), 2.45-2.60 (m, 2H), 2.31-2.41 (m, 2H).

### Example 54

### N-(3-Chlorobenzyl)-2-[2-(diethylamino)-2-phenylethyl]-2H-indazole-6-carboxamide

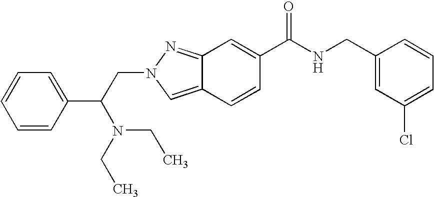

Similarly to the preparation procedure in Example 6, 60.79 mg (0.138 mmol) of the aldehyde from Example 6A is reacted with 34.4 mg (0.179 mmol) N¹,N¹-diethyl-1-phenylethane-1,2-diamine to the corresponding indazole derivative. We obtain, after purification by preparative HPLC, 26 mg (41% of th.) of product.

LCMS (method 8): Rₜ=1.57 min (m/z=461 (M+H)⁺)

¹H-NMR (400 MHz, DMSO-d₆): δ=9.08 (t, 1H), 8.29 (s, 1H), 8.18 (s, 1H), 7.70 (d, 1H), 7.47 (d, 1H), 7.22-7.40 (m, 9H), 5.0-5.1 (m, 1H), 4.79-4.91 (m, 1H), 4.53-4.65 (m, 1H), 4.48 (d, 2H), 3.99-4.09 (m, 2H), 3.82-3.92 (m, 2H), 1.25 (t, 3H), 1.20 (t, 3H).

### Example 55

### N-(3-Chlorobenzyl)-2-[2-(4-methoxyphenyl)-2-pyrrolidin-1-ylethyl]-2H-indazole-6-carboxamide

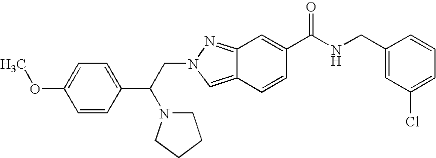

Similarly to the preparation procedure in Example 6, 60.79 mg (0.138 mmol) of the aldehyde from Example 6A is reacted with 39.44 mg (0.179 mmol) of 2-(4-methoxyphenyl)-2-pyrrolidin-1-ylethanamine to the corresponding indazole derivative. We obtain, after purification by preparative HPLC, 19 mg (27% of th.) of product.

LCMS (method 5): Rₜ=1.61 min (m/z=489 (M+H)⁺)

¹H-NMR (400 MHz, DMSO-d₆): δ=9.11 (t, 1H), 8.16 (s, 1H), 8.09 (s, 1H), 7.66 (d, 1H), 7.45 (d, 1H), 7.28-7.40 (m, 4H), 7.09-7.19 (m, 2H), 6.77 (d, 2H), 4.92-5.07 (m, 1H), 4.68-4.82 (m, 1H), 4.49 (d, 2H), 4.0-4.08 (m, 1H), 3.67 (s, 3H), 2.3-2.6 (m, 4H), 1.6-1.75 (m, 4H).

### Example 56

### N-(3-Chlorobenzyl)-2-[2-(4-fluorophenyl)-2-(4-methylpiperazin-1-yl)ethyl]-2H-indazole-6-carboxamide

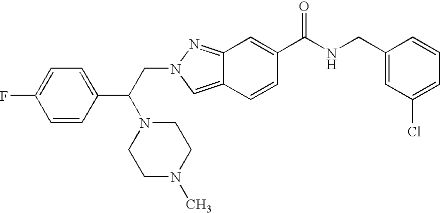

Similarly to the preparation procedure in Example 6, 60.79 mg (0.138 mmol) of the aldehyde from Example 6A is reacted with 42.48 mg (0.179 mmol) of 2-(4-fluorophenyl)-2-(4-methylpiperazin-1-yl)ethanamine to the corresponding indazole derivative. We obtain, after purification by preparative HPLC, 10 mg (11% of th.) of product.

LCMS (method 8): Rₜ=1.61 min (m/z=506 (M+H)⁺)

¹H-NMR (400 MHz, DMSO-d₆): δ=9.13 (t, 1H), 8.34 (s, 1H), 8.17 (s, 1H), 7.73 (d, 1H), 7.50 (d, 2H), 7.25-7.42 (m, 5H), 7.19 (t, 2H), 5.09 (dd, 1H), 4.85 (dd, 1H), 4.43-4.53 (m, 3H), 3.99-4.08 (m, 4H), 3.82-3.9 (m, 3H), 3.14-3.23 (m, 1H), 2.67 (s, 3H).

### Example 57

### N-(3-Chlorobenzyl)-2-(2,3-dipyridin-2-ylpropyl)-2H-indazole-6-carboxamide

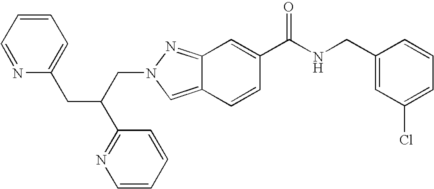

Similarly to the preparation procedure in Example 6, 70 mg (0.187 mmol) of the aldehyde from Example 6A is reacted with 159.3 mg (0.75 mmol) 2,3-dipyridin-2-ylpropan-1-amine to the corresponding indazole derivative. We obtain, after purification by preparative HPLC, 4 mg (4% of th.) of product.

LCMS (method 5): Rₜ=1.82 min (m/z=482 (M+H)⁺)

¹H-NMR (400 MHz, DMSO-d₆): δ=9.09 (t, 1H), 8.53 (d, 1H), 8.44 (d, 1H), 8.19 (d, 2H), 7.66 (d, 1H), 7.55 (dd, 1H), 7.43-7.5 (m, 2H), 7.25-7.4 (m, 4H), 7.09-7.16 (m, 2H), 7.01 (d, 1H), 6.90 (d, 1H), 4.91 (dd, 1H), 4.78 (dd, 1H), 4.48 (d, 2H), 4.12-4.21 (m, 1H), 3.25 (dd, 1H), 3.05 (dd, 1H).

### Example 58

### N-(3-Chlorobenzyl)-2-[2-(4-fluorophenyl)-2-pyrrolidin-1-ylethyl]-2H-indazole-6-carboxamide

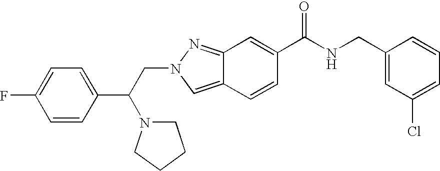

Similarly to the preparation procedure in Example 6, 60.79 mg (0.138 mmol) of the aldehyde from Example 6A is reacted with 208.3 mg (0.179 mmol) of 2-(4-fluorophenyl)-2-pyrrolidin-1-ylethanamine to the corresponding indazole derivative. We obtain, after purification by preparative HPLC, 29 mg (44% of th.) of product.

LCMS (method 7): Rₜ=2.49 min (m/z=477 (M+H)⁺)

¹H-NMR (400 MHz, DMSO-d₆): δ=9.10 (t, 1H), 8.15 (s, 1H), 8.06 (s, 1H), 7.65 (d, 1H), 7.45 (dd, 1H), 7.36-7.40 (m, 2H), 7.27-7.33 (m, 2H), 7.16-7.24 (m, 2H), 7.02 (t, 2H), 4.99 (dd, 1H), 4.71 (dd, 1H), 4.48 (d, 2H), 3.95-4.13 (m, 1H), 2.53-2.62 (m, 4H), 2.37-2.45 (m, 4H).

### Example 59

### N-(3-Chlorobenzyl)-2-[2-(dimethylamino)-2-(4-methylphenyl)ethyl]-2H-indazole-6-carboxamide

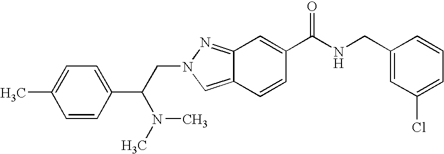

Similarly to the preparation procedure in Example 6, 145.73 mg (0.33 mmol) of the aldehyde from Example 6A is reacted with 178.3 mg (0.43 mmol) of N¹,N¹-dimethyl-1-(4-methylphenyl)ethane-1,2-diamine to the corresponding indazole derivative. We obtain, after purification by preparative HPLC, 29.5 mg (17% of th.) of product.

LCMS (method 5): Rₜ=1.63 min (m/z=447 (M+H)⁺)

¹H-NMR (400 MHz, DMSO-d₆): δ=9.09 (t, 1H), 8.27 (s, 1H), 8.17 (s, 1H), 7.70 (d, 1H), 7.47 (dd, 1H), 7.34-7.41 (m, 2H), 7.27-7.33 (m, 2H), 7.16 (d, 2H), 7.10 (d, 2H), 5.01 (dd, 1H), 4.76 (dd, 1H), 4.48 (m, 3H), 2.25 (s, 3H), 2.10 (s, 6H).

### Example 60

### N-{5-Chloro-2-[2-(cyclopropylamino)-2-oxothoxy]benzyl}-2-[2-(1H-pyrazol-1-yl)ethyl]-2H-indazole-6-carboxamide

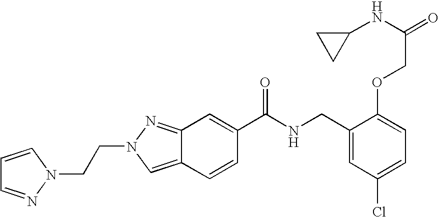

23 mg (0.09 mmol) of the carboxylic acid from Example 13A is put in DMF and 25.8 mg (0.134 mmol) EDC and 13.3 mg (0.1 mmol) HOBt are added. Then it is stirred for 2 h at RT. Then 43 mg (0.12 mmol) of 2-[2-(aminomethyl)-4-chlorphenoxy]-N-cyclopropyl-acetamide, which can be prepared following the instructions described in WO 98/31670, is added and the solution is stirred for 16 h at RT. We obtain, after purification by preparative HPLC, 21.5 mg (49% of th.) of product.

MS (ESIpos): m/z=493 (M+H)⁺.

¹H-NMR (400 MHz, DMSO-d₆): δ=9.0 (t, 1H), 8.21 (s, 2H), 8.14 (s, 1H), 7.71 (d, 2H), 7.49 (dd, 1H), 7.41 (dd, 1H), 7.27-7.32 (m, 2H), 6.92-6.98 (m, 1H), 6.11 (t, 1H), 4.90 (t, 2H), 4.71 (t, 2H), 4.55 (d, 2H), 4.52 (s, 2H), 2.7-2.8 (m, 1H), 0.56-0.71 (m, 4H).

### Example 61

### Ethyl(4-chloro-2-{[({2-[2-(1H-pyrazol-1-yl)ethyl]-2H-indazol-6-ylγcarbonyl)amino]methyl}-phenoxy)acetate

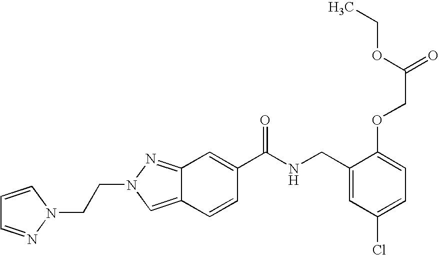

Similarly to the preparation procedure in Example 60, 23 mg (0.09 mmol) of the acid from Example 13A is reacted with 41.7 mg (0.12 mmol) of ethyl[2-(aminomethyl)-4-chlorphenoxy]acetate, which can be prepared following the instructions described in WO 98/31670, to the corresponding amide. We obtain, after purification by preparative HPLC, 8 mg (18% of th.) of product.

LCMS (method 1): Rₜ=2.03 min (m/z=482 (M+H)⁺)

¹H-NMR (400 MHz, cDCl₃): δ=8.21 (s, 1H), 7.49-7.58 (m, 3H), 7.46 (s, 1H), 7.4-7.45 (m, 2H), 7.21 (dd, 1H), 6.82 (d, 1H), 6.73 (d, 1H), 6.04 (t, 1H), 4.87 (t, 2H), 4.66-4.75 (m, 5H), 4.27 (q, 2H), 1.82-1.88 (m, 1H), 1.28 (t, 3H).

### Example 62

### N-(3-Chlorobenzyl)-2-[2-(2-methoxyphenyl)-2-morpholin-4-ylethyl]-2H-indazole-6-carboxamide trifluoracetate

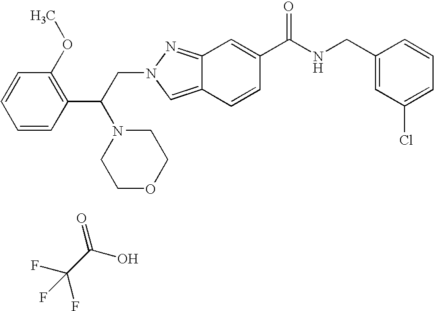

Similarly to the preparation procedure in Example 6, 19.6 mg (0.058 mmol) of the aldehyde from Example 6A is reacted with 31 mg (0.075 mmol) of 2-(2-methoxyphenyl)-2-morpholin-4-ylethanamine to the corresponding indazole derivative. We obtain, after purification by preparative HPLC, 3 mg (8% of th.) of product as a solid.

LCMS (method 7): Rₜ=2.56 min (m/z=505 (M+H)⁺)

### Example 63

### N-[(5-Chloro-2-thienyemethyl]-2-(2-ethylbutyl)-2H-indazole-6-carboxamide

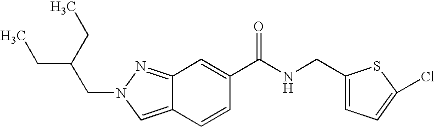

Similarly to the preparation procedure in Example 36, 20 mg (0.081 mmol) of the acid from Example 26A is reacted with 15.6 mg (0.11 mmol) of 1-(5-chloro-2-thienyl)methanamine to the corresponding amide. We obtain, after purification by preparative HPLC, 21.3 mg (70% of th.) of product.

MS (ESIpos): m/z=376 (M+H)⁺.

¹H-NMR (400 MHz, DMSO-d₆): δ=9.21 (t, 1H), 8.45 (s, 1H), 8.17 (s, 1H), 7.76 (d, 1H), 7.49 (d, 1H), 6.96 (d, 1H), 6.90 (d, 1H), 4.55 (d, 2H), 4.36 (d, 2H), 1.9-2.01 (m, 1H), 1.18-1.29 (m, 4H), 0.86 (t, 6H).

### Example 64

### 2-(2-Azepan-1-yl-2-phenylethyl)-N-(3-chlorobenzyl)-2H-indazole-6-carboxamide

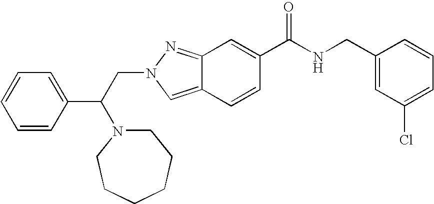

Similarly to the preparation procedure in Example 6, 60.79 mg (0.138 mmol) of the aldehyde from Example 6A is reacted with 39.1 mg (0.179 mmol) of 2-azepan-1-yl-2-phenylethanamine to the corresponding indazole derivative. We obtain, after purification by preparative HPLC, 13 mg (19% of th.) of product.

LCMS (method 7): Rₜ=2.54 min (m/z=487 (M+H)⁺)

¹H-NMR (400 MHz, DMSO-d₆): δ=9.10 (t, 1H), 8.35 (s, 1H), 8.19 (s, 1H), 7.72 (d, 1H), 7.48 (dd, 1H), 7.2-7.4 (m, 9H), 5.01 (dd, 1H), 4.82 (dd, 1H), 4.46-4.53 (m, 3H), 2.67-2.78 (m, 2H), 2.4-2.5 (m, 2H), 1.3-1.5 (m, 8H).

### Example 65

### N-[(5-Chloro-2-thienyl)methyl]-2-[2-(1H-pyrazol-1-yl)ethyl]-2H-indazole-6-carboxamide

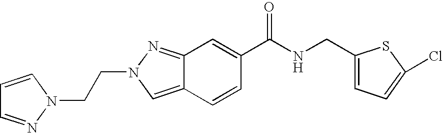

Similarly to the preparation procedure in Example 36, 50 mg (0.20 mmol) of the acid from Example 13A is reacted with 40.3 mg (0.27 mmol) of 1-(5-chloro-2-thienyl)methanamine to the corresponding amide. We obtain, after purification by preparative HPLC, 45.5 mg (60% of th.) of product.

MS (ESIpos): m/z=386 (M+H)⁺.

¹H-NMR (400 MHz, DMSO-d₆): δ=9.22 (t, 1H), 8.14 (d, 2H), 7.70 (d, 1H), 7.47 (d, 1H), 7.41 (d, 2H), 6.96 (d, 1H), 6.90 (d, 1H), 6.11 (s, 1H), 4.90 (t, 2H), 4.71 (t, 2H), 4.55 (d, 2H).

### Example 66

### N-(3-Chlorobenzyl)-2-[4-(2-oxopyrrolidin-1-yl)-2-pyridin-2-ylbutyl]-2H-indazole-6-carboxamide

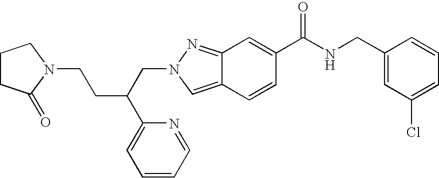

Similarly to the preparation procedure in Example 6, 70 mg (0.187 mmol) of the aldehyde from Example 6A is reacted with 174.2 mg (0.75 mmol) of 1-(4-amino-3-pyridin-2-ylbutyl)pyrrolidin-2-one to the corresponding indazole derivative. We obtain, after purification by preparative HPLC, 4 mg (4% of th.) of product.

LCMS (method 1): Rₜ=1.75 min (m/z=502 (M+H)⁺)

### Example 67

### N-(3-Chlorobenzyl)-2-[3-(4-methylpiperazin-1-yl)-3-oxo-2-phenylpropyl]-2H-indazole-6-carboxamide

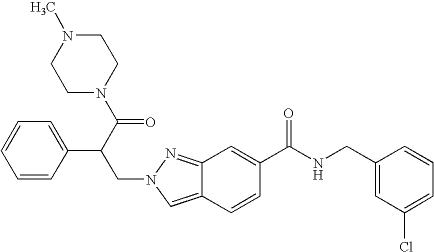

Similarly to the preparation procedure in Example 28, 35 mg (0.08 mmol) of the acid from Example 27 is reacted with 11.3 mg (0.11 mmol) 1-methylpiperazine to the corresponding amide. We obtain, after purification by preparative HPLC, 10 mg (23% of th.) of product.

LCMS (method 5): Rₜ=1.53 min (m/z=516 (M+H)⁺)

¹H-NMR (400 MHz, cDCl₃): δ=8.17 (s, 1H), 7.82 (s, 1H), 7.63 (d, 1H), 7.44 (d, 1H), 7.37 (s, 1H), 7.17-7.33 (m, 8H), 6.48 (t, 1H), 5.16 (dd, 1H), 4.67 (d, 2H), 4.62 (d, 1H), 4.58 (t, 1H), 3.63 (m, 1H), 3.43-3.53 (m, 1H), 3.3-3.4 (m, 1H), 3.2-3.29 (m, 1H), 2.15-2.3 (m, 2H), 2.12 (s, 3H), 1.55-1.8 (m, 2H).

### Example 68

### N-(3-Chlorobenzyl)-2-[3-(4-hydroxypiperidin-1-yl)-3-oxo-2-phenylpropyl]-2H-indazole-6-carboxamide

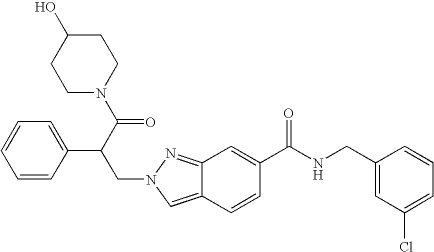

Similarly to the preparation procedure in Example 28, 35 mg (0.08 mmol) of the acid from Example 27 is reacted with 12.2 mg (0.12 mmol) piperidin-4-ol to the corresponding amide. We obtain, after purification by preparative HPLC, 15 mg (36% of th.) of product.

LCMS (method 5): Rₜ=2.24 min (m/z=517 (M+H)⁺)

¹H-NMR (400 MHz, DMSO-d₆): δ=9.12 (t, 1H), 8.24 (s, 1H), 8.18 (s, 1H), 7.70 (d, 1H), 7.48 (d, 1H), 7.24-7.41 (m, 9H), 5.03 (dd, 1H), 4.84 (dt, 1H), 4.56-4.67 (m, 2H), 4.50 (d, 2H), 3.92-4.02 (m, 1H), 3.59-3.76 (m, 1H), 3.44-3.59 (m, 1H), 2.92-3.18 (m, 2H), 1.32-1.6 (m, 2H), 1.08-1.2 (m, 2H).

### Example 69

### Ethyl-1-(3-{6-[(3-chlorobenzyl)carbamoyl]-2H-indazol-2-yl}-2-phenylpropanoyl)piperidine-4-carboxylate

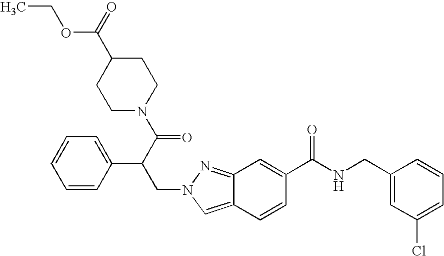

Similarly to the preparation procedure in Example 28, 35 mg (0.08 mmol) of the acid from Example 27 is reacted with 19 mg (0.12 mmol) ethyl-piperidine-4-carboxylate to the corresponding amide. We obtain, after purification by preparative HPLC, 30 mg (65% of th.) of product.

LCMS (method 5): Rₜ=2.67 min (m/z=573 (M+H)⁺)

¹H-NMR (400 MHz, DMSO-d₆): δ=9.1 (dt, 1H), 8.24 (d, 1H), 8.18 (s, 1H), 7.70 (d, 1H), 7.48 (d, 1H), 7.21-7.41 (m, 9H), 5.06 (dt, 1H), 4.86 (dt, 1H), 4.63 (dt, 1H), 4.50 (d, 2H), 4.15 (dd, 1H), 3.96 (dq, 2H), 3.85 (dd, 1H), 2.9 (dt, 1H), 2.6-2.72 (m, 1H), 2.40-2.48 (m, 1H), 1.4-1.75 (m, 2H), 1.12-1.38 (m, 2H), 1.09 (t, 3H).

### Example 70

### 1-(3-{6-[(3-Chlorobenzyl)carbamoyl]-2H-indazol-2-yl}-2-phenylpropanoyl)piperidine-4-carboxylic acid

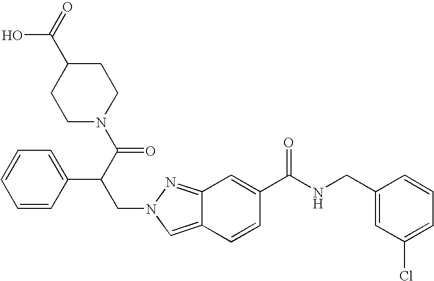

Similarly to the preparation procedure in Example 13A, 28 mg (0.049 mmol) of the ester from Example 69 is saponified with lithium hydroxide to the corresponding acid. We obtain, after purification by preparative HPLC, 26 mg (99% of th.) of product.

LCMS (method 7): Rₜ=3.39 min (m/z=545 (M+H)⁺)

¹H-NMR (400 MHz, DMSO-d₆): δ=12.1 (s, br, 1H), 9.11 (dt, 1H), 8.24 (d, 1H), 8.18 (s, 1H), 7.70 (dd, 1H), 7.48 (d, 1H), 7.21-7.41 (m, 9H), 5.04 (dd, 1H), 4.84 (dt, 1H), 4.57-4.69 (m, 1H), 4.50 (d, 2H), 4.18 (dd, 1H), 2.59-3.02 (m, 2H), 2.28-2.41 (m, 1H), 1.63-1.75 (m, 1H), 1.04-1.58 (m, 4H).

### B) ASSESSMENT OF PHYSIOLOGICAL EFFICACY

The suitability of the compounds according to the invention for the treatment of thromboembolic diseases can be shown in following assay systems:

In vitro Enzyme Assay

Measurement of Thrombin Inhibition

For determination of the thrombin inhibition of the aforementioned substances, a biochemical test system is used, in which the conversion of a thrombin substrate is used for determining the enzymatic activity of human thrombin. In this, thrombin cleaves aminomethylcoumarin from the peptic substrate, and this is measured by fluorescence. The determinations are carried out in microtitre plates.

The test substances are dissolved at different concentrations in dimethylsulphoxide and incubated for 15 min with human thrombin (0.06 nmol/l dissolved in 50 mmol/l Tris-buffer [C,C,C-Tris(hydroxymethyl)-aminomethane], 100 mmol/l NaCl, 0.1% BSA [bovine serum albumin], pH 7.4) at 22° C. Then the substrate (5 μmol/l Boc-Asp(OBzl)-Pro-Arg-AMC from the company Bachem) is added. After incubation for 30 min the sample is excited at a wavelength of 360 nm and the emission at 460 nm is measured. The measured emissions of the test preparations with test substance are compared with the control preparations without test substance (dimethylsulphoxide only, instead of test substance in dimethylsulphoxide) and IC₅₀ values are calculated from the concentration-effect relations.

TABLE A

| Example No. | IC₅₀ [nM] |
|---|---|
| 4 | 216 |
| 26 | 110 |
| 49 | 26 |

Determination of Selectivity

To demonstrate the selectivity of the substances with respect to thrombin inhibition, the test substances are investigated for their inhibition of other human serine proteases such as factor Xa, factor XIa, trypsin and plasmin. For determination of the enzymatic activity of factor Xa (1.3 nmol/l from Kordia), factor XIa (0.4 nmol/l from Kordia), trypsin (83 mU/ml from Sigma) and plasmin (0.1 μg/ml from Kordia) these enzymes are dissolved (50 mmol/l Tris-buffer [C,C,C-Tris(hydroxymethyl)-aminomethane], 100 mmol/l NaCl, 0.1% BSA [bovine serum albumin], 5 mmol/l calcium chloride, pH 7.4) and incubated for 15 min with the test substance at various concentrations in dimethylsulphoxide and with dimethylsulphoxide without the test substance. Then the enzymatic reaction is started by adding the corresponding substrates (5 μmol/l Boc-Ile-Glu-Gly-Arg-AMC from Bachem for factor Xa and trypsin, 5 μmol/l Boc-Glu(OBzl)-Ala-Arg-AMC from Bachem for factor XIa, 50 μmol/l MeOSuc-Ala-Phe-Lys-AMC from Bachem for plasmin). After an incubation time of 30 min at 22° C., the fluorescence is measured (excitation: 360 nm, emission: 460 nm). The measured emissions of the test preparations with the test substance are compared with the control preparations without test substance (dimethylsulphoxide only, instead of test substance in dimethylsulphoxide) and IC₅₀ values are calculated from the concentration-effect relations.

Thrombin Plasma Assay

In a 96-well flat-bottom plate, 20 μl substance dilution (in water) is mixed with 20 μl Ecarin (Ecarin Reagent, from Sigma E-0504, final conc. 20 mU/ml, 20 mU final concentration in the well) in Ca-buffer (200 mM Hepes+560 mM NaCl+10 mM CaCl₂+0.4% PEG). In the first upper 3 wells A1-A3 only Ca-buffer is added, these samples serve as unstimulated controls. In addition, 20 μl fluorogenic thrombin substrate (from Bachem I-1120, 50 μM final conc. in the well) and 20 μl citrate plasma (from Octapharma) are added to each well and homogenized well. The plate is measured in the Spectra fluor plus Reader with an excitation filter 360 nm and emission filter 465 nm each minute over a period of 20 min. The IC₅₀ value is determined after approx. 12 minutes, when 70% of the maximum value has been reached.

Thrombin Generation Assay (Thrombogram)

The effect of the test substances on the thrombogram (Thrombin Generation Assay according to Hemker) is determined in vitro in human plasma (Octaplas® from the company Octapharma).

In the Thrombin Generation Assay according to Hemker, the activity of thrombin in coagulating plasma is determined by measurement of the fluorescent cleavage products of the substrate I-1140 (Z-Gly-Gly-Arg-AMC, Bachem). The reactions are carried out in the presence of varying concentrations of test substance or the corresponding solvent. The reaction is started using reagents from the company Thrombinoscope (PPP reagent: 30 μM recombinant tissue factor, 24 μM phospholipids in HEPES). In addition, a Thrombin Calibrator from the company Thrombinoscope is used, whose amidolytic activity is required for calculation of the thrombin activity in a sample with unknown amount of thrombin. The test is carried out according to the manufacturer's instructions (Thrombinoscope BV): 4 μl of the test substance or of the solvent, 76 μl plasma and 20 μl PPP reagent or Thrombin Calibrator are incubated for 5 min at 37° C. After adding 20 μl 2.5 mM thrombin substrate in 20 mM Hepes, 60 mg/ml BSA, and 102 mM CaCl₂, thrombin generation is measured every 20 s for 120 min. The measurement is carried out with a fluorometer (Fluoroskan Ascent) from the company Thermo Electron, which is equipped with a 390/460 nM filter pair and a dispenser.

Using the “thrombinoscope software”, the thrombogram is calculated and presented graphically. The following parameters are calculated: lag time, time to peak, peak, ETP (endogenous thrombin potential) and start tail.

Determination of Anticoagulant Action

The anticoagulant action of the test substances is determined in vitro in human, rabbit and rat plasma. For this, blood is taken as an initial sample in a mixture ratio sodium citrate/blood of 1/9, using a 0.11 molar sodium citrate solution Immediately after it is obtained, the blood is mixed well and centrifuged for 15 minutes at approx. 4000 g. The supernatant is pipetted off.

The prothrombin time (PT, synonyms: thromboplastin time, Quick-Test) is determined in the presence of varying concentrations of test substance or the corresponding solvent with a commercially available test kit (Neoplastin® from the company Boehringer Mannheim or Hemoliance® RecombiPlastin from the company Instrumentation Laboratory). The test compounds are incubated with the plasma for 3 minutes at 37° C. Then coagulation is initiated by adding thromboplastin and the time of onset of coagulation is determined The concentration of test substance that gives rise to a doubling of the prothrombin time is determined.

The thrombin time (TT) is determined in the presence of varying concentrations of test substance or the corresponding solvent with a commercially available test kit (Thrombin Reagent from the company Roche). The test compounds are incubated with the plasma for 3 minutes at 37° C. Then coagulation is initiated by adding the Thrombin Reagent and the time of onset of coagulation is determined The concentration of test substance that gives rise to a doubling of the thrombin time is determined.

The activated partial thromboplastin time (APTT) is determined in the presence of varying concentrations of test substance or the corresponding solvent with a commercially available test kit (PTT reagent from the company Roche). The test compounds are incubated with the plasma and the PTT reagent (cephalin, kaolin) for 3 minutes at 37° C. Then coagulation is initiated by adding 25 mM CaCl₂ and the time of onset of coagulation is determined The concentration of test substance that gives rise to a doubling of APTT is determined.

Venous Stasis Model (Rat)

Fasting male rats (strain: HSD CPB:WU) with a weight of 200-250 g are anaesthetized with Rompun/Ketavet solution (12 mg/kg/50 mg/kg) or with inactin (150-180 mg/kg). Thrombus formation is induced in a clamped segment of the vena cava by the method described by S. Wessler et al. in J. Appl. Physiol (1959), 14, 943-946. For this, thromboplastin (Neoplastin Plus, Diagnostica Stago, 0.5 mg/kg) is injected through a catheter into the vena femoralis immediately before the induction of stasis. 10-15 seconds after thromboplastin injection, the vena cava is tied off with ligatures 0.8-1 cm apart. 15 minutes after thromboplastin injection, the thrombi are removed and weighed. Before setting up the extracorporeal circulation, the test substances are administered to the conscious animals either intravenously via the caudal or penile vein or orally by stomach tube.

Arteriovenous Shunt Model (Rat)

Fasting male rats (strain: HSD CPB:WU) with a weight of 200-250 g are anaesthetized with Rompun/Ketavet solution (12 mg/kg/50 mg/kg) or with inactin (150-180 mg/kg). Thrombus formation is induced in an arteriovenous shunt by the method described by Christopher N. Berry et al., Br. J. Pharmacol. (1994), 113, 1209-1214. For this, the left vena jugularis and the right arteria carotis are exposed. An extracorporeal shunt is applied between the two vessels with a 10 cm long polyethylene tube (PE 60). This polyethylene tube was joined in the middle to another 3 cm long polyethylene tube (PE 160), which contained a roughened nylon thread arranged in a loop, for the production of a thrombogenic surface. Extracorporeal circulation is maintained for 15 minutes. Then the shunt is removed and the nylon thread with the thrombus is weighed immediately. The empty weight of the nylon thread was determined before the start of the test. Before setting up the extracorporeal circulation, the test substances are administered to the conscious animals either intravenously via the caudal or penile vein or orally by stomach tube.

### C) EXAMPLES OF APPLICATION FOR PHARMACEUTICAL COMPOSITIONS

The substances according to the invention can be converted into pharmaceutical preparations as follows:

Tablet:

Composition:

100 mg of the compound from Example 1, 50 mg lactose (monohydrate), 50 mg maize starch, 10 mg polyvinylpyrrolidone (PVP 25) (from BASF, Germany) and 2 mg magnesium stearate.

Tablet weight 212 mg. Diameter 8 mm, convexity radius 12 mm.

Production:

The mixture of the compound from Example 1, lactose and starch is granulated with a 5% solution (w/w) of PVP in water. After drying, the granules are mixed with the magnesium stearate for 5 min. This mixture is compacted in an ordinary tablet press (for tablet format, see above).

Oral Suspension:

Composition:

1000 mg of the compound from Example 1, 1000 mg ethanol (96%), 400 mg Rhodigel (xanthan gum) (from FMC, USA) and 99 g water.

A single dose of 100 mg of the compound according to the invention corresponds to 10 ml oral suspension.

Production:

The Rhodigel is suspended in ethanol, and the compound from Example 1 is added to the suspension. Water is added, while stirring. It is stirred for approx. 6 h, until swelling of the Rhodigel ceases.

Solution for Intravenous Application:

Composition:

1 mg of the compound from Example 1, 15 g polyethylene glycol 400 and 250 g water for injection.

Production:

The compound from Example 1 together with polyethylene glycol 400 is dissolved in the water, while stirring. The solution is sterile-filtered (pore diameter 0.22 μm) and heat-sterilized infusion vials are filled under aseptic conditions. The vials are sealed with infusion stoppers and crimp caps.

We claim:

1. A compound of formula (I): 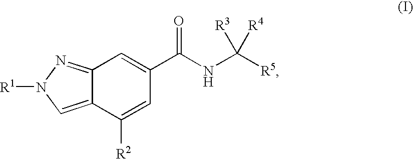
and
in which
R¹ stands for a compound of formula 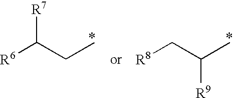
where * is the site of linkage to the indazole,
R⁶ stands for C₁-C₆-alkyl, C₃-C₈-cycloalkyl, phenyl, 5- to 7-membered heterocyclyl or 5- or 6-membered heteroaryl,
in which cycloalkyl and heterocyclyl can be substituted with 1 to 3 substituents, the substituents being selected independently of one another from the group comprising halogen, oxo, C₁-C₄-alkyl, C₁-C₄-alkoxy, C₁-C₄-alkylthio, C₁-C₄-alkyl-amino, C₁-C₄-alkylcarbonyl and C₁-C₄-alkoxycarbonyl,
and
in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
R⁷ stands for hydrogen, C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
in which cycloalkyl and heterocyclyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
and
in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
and
in which heterocyclyl and heterocyclylcarbonyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
and
in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
R⁸ stands for C₁-C₆-alkyl, C₃-C₈-cycloalkyl, phenyl, 5- to 7-membered heterocyclyl or 5- or 6-membered heteroaryl,
in which cycloalkyl and heterocyclyl can be substituted with 1 to 3 substituents, the substituents being selected independently of one another from the group comprising halogen, oxo, C₁-C₄-alkoxy, C₁-C₄-alkylthio, C₁-C₄-alkylamino, C₁-C₄-alkylcarbonyl and C₁-C₄-alkoxycarbonyl,
and
in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
R⁹ stands for C₁-C₆-alkyl, C₁-C₄-alkylamino, C₁-C₄-alkoxycarbonyl, 5- to 7-membered heterocyclyl, 5- to 7-membered heterocyclylcarbonyl or 5- or 6-membered heteroaryl,
in which alkyl can be substituted with a substituent, the substituent being selected from the group comprising hydroxy, C₁-C₄-alkoxy, C₁-C₄-alkylamino, C₁-C₄-alkylthio, C₁-C₄-alkylcarbonyl, C₁-C₄-alkoxycarbonyl, C₁-C₄-alkylaminocarbonyl, C₁-C₄-alkylcarbonylamino, C₃-C₈-cycloalkyl, phenyl, 5- to 7-membered heterocyclyl and 5- to 6-membered heteroaryl,
in which cycloalkyl and heterocyclyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
and
in which phenyl and heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxycarbonyl,
and
in which heterocyclyl and heterocyclylcarbonyl can be substituted with 1 to 3 substituents, the substituents being selected independently of one another from the group comprising halogen, hydroxy, amino, hydroxycarbonyl, aminocarbonyl, oxo, C₁-C₄-alkyl, C₁-C₄-alkoxy, C₁-C₄-alkylthio, C₁-C₄-alkylamino, C₁-C₄-alkylcarbonyl, C₁-C₄-alkoxycarbonyl and C₁-C₄-alkylaminocarbonyl,
and
in which heteroaryl can be substituted with 1 to 3 substituents, the substituents being selected independently of one another from the group comprising halogen, cyano, trifluoromethyl, trifluoromethoxy, trifluoromethylthio, C₁-C₄-alkyl, C₁-C₄-alkoxy, C₁-C₄-alkylthio, C₁-C₄-alkylamino, C₁-C₄-alkylcarbonyl and C₁-C₄-alkoxy-carbonyl,
R² stands for hydrogen, halogen, cyano, trifluoromethyl, trifluoromethoxy, trifluoromethylthio, C₁-C₃-alkyl, C₁-C₃-alkoxy, C₁-C₃-alkylthio or cyclopropyl,
in which alkyl, alkoxy, alkylthio and cyclopropyl can be substituted with 1 to 3 substituents, the substituents being selected independently of one another from the group comprising halogen,
R³ stands for hydrogen or C₁-C₄-alkyl,
R⁴ stands for hydrogen or C₁-C₄-alkyl,
or
R³ and R⁴ form, together with the carbon atom to which they are bound, a cyclopropyl ring or a cyclobutyl ring,
R⁵ stands for phenyl, 2-thienyl or 3-thienyl,
in which phenyl can be substituted with 1 to 3 substituents, the substituents being selected independently of one another from the group comprising halogen, methyl, ethinyl, methoxy and 1,2,4-triazol-1-yl,
in which methoxy can be substituted with a substituent, the substituent being selected from the group comprising C₁-C₄-alkoxycarbonyl, C₁-C₄-alkylaminocarbonyl and C₃-C₆-cycloalkylaminocarbonyl,
and
in which 2-thienyl and 3-thienyl can be substituted with 1 to 3 substituents, the substituents being selected independently of one another from the group comprising halogen, methyl, ethinyl and methoxy,
or one of their salts, their solvates or the solvates of their salts.

2. The compound according to claim 1, characterized in that
R¹ stands for a compound of formula 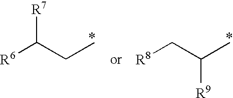
where * is the site of linkage to the indazole,
R⁶ stands for C₁-C₆-alkyl, phenyl, 5- to 7-membered heterocyclyl or 5- or 6-membered heteroaryl,
in which heterocyclyl can be substituted with 1 to 3 substituents, the substituents being selected independently of one another from the group comprising halogen, oxo, C₁-C₄-alkyl, C₁-C₄-alkoxy and C₁-C₄-alkylamino,
and
in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylamino and C₁-C₄-alkylaminocarbonyl,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with 1 to 3 substituents, the substituents being selected independently of one another from the group comprising halogen, oxo, C₁-C₄-alkyl, C₁-C₄-alkoxy, C₁-C₄ alkylamino, C₁-C₄-alkylcarbonyl and C₁-C₄-alkoxycarbonyl,
and
in which phenyl and heteroaryl can be substituted with 1 to 3 substituents, the substituents being selected independently of one another from the group comprising halogen, cyano, trifluoromethyl, trifluoromethoxy, C₁-C₄-alkyl, C₁-C₄-alkoxy, C₁-C₄-alkylamino, C₁-C₄-alkylcarbonyl and C₁-C₄-alkoxycarbonyl,
and
in which heterocyclyl and heterocyclylcarbonyl can be substituted with 1 to 3 substituents, the substituents being selected independently of one another from the group comprising halogen, oxo, C₁-C₄-alkyl, C₁-C₄-alkoxy, C₁-C₄ alkylamino, C₁-C₄-alkylcarbonyl and C₁-C₄-alkoxycarbonyl,
R⁸ stands for C₁-C₆-alkyl, phenyl, 5- to 7-membered heterocyclyl or 5- or 6-membered heteroaryl,
in which heterocyclyl can be substituted with 1 to 3 substituents, the substituents being selected independently of one another from the group comprising halogen, oxo, C₁-C₄-alkoxy and C₁-C₄-alkylamino,
and
in which phenyl and heteroaryl can be substituted with 1 to 3 substituents, the substituents being selected independently of one another from the group comprising halogen, cyano, trifluoromethyl, trifluoromethoxy, aminocarbonyl, C₁-C₄-alkyl, C₁-C₄-alkoxy, C₁-C₄-alkylamino and C₁-C₄-alkylaminocarbonyl,
R⁹ stands for C₁-C₆-alkyl, C₁-C₄ alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with 1 to 3 substituents, the substituents being selected independently of one another from the group comprising halogen, oxo, C₁-C₄-alkyl, C₁-C₄-alkoxy, C₁-C₄ alkylamino, C₁-C₄-alkylcarbonyl and C₁-C₄-alkoxycarbonyl,
and
in which phenyl and heteroaryl can be substituted with 1 to 3 substituents, the substituents being selected independently of one another from the group comprising halogen, cyano, trifluoromethyl, trifluoromethoxy, C₁-C₄-alkyl, C₁-C₄-alkoxy, C₁-C₄-alkylamino, C₁-C₄-alkylcarbonyl and C₁-C₄-alkoxycarbonyl,
and
in which heterocyclyl and heterocyclylcarbonyl can be substituted with 1 to 3 substituents, the substituents being selected independently of one another from the group comprising halogen, oxo, C₁-C₄-alkyl, C₁-C₄-alkoxy, C₁-C₄ alkylamino, C₁-C₄-alkylcarbonyl and C₁-C₄-alkoxycarbonyl,
R² stands for hydrogen, halogen, cyano, trifluoromethyl, trifluoromethoxy, trifluoromethylthio, C₁-C₃-alkyl, C₁-C₃-alkoxy, C₁-C₃-alkylthio or cyclopropyl,
R³ stands for hydrogen or methyl,
R⁴ stands for hydrogen or methyl,
or
R³ and R⁴ form, together with the carbon atom to which they are bound, a cyclopropyl ring,
R⁵ stands for phenyl, 2-thienyl or 3-thienyl,
in which phenyl, 2-thienyl and 3-thienyl can be substituted with 1 to 3 substituents, the substituents being selected independently of one another from the group comprising halogen, methyl, ethinyl and methoxy,
or one of their salts, their solvates or the solvates of their salts.

3. The compound according to claim 1, characterized in that
R¹ stands for a compound of formula 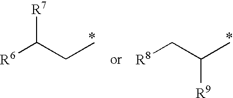
where * is the site of linkage to the indazole,
R⁶ stands for C₁-C₆-alkyl, phenyl, 5- to 7-membered heterocyclyl or 5- or 6-membered heteroaryl,
in which heterocyclyl can be substituted with 1 to 3 substituents, the substituents being selected independently of one another from the group comprising oxo and C₁-C₄-alkyl,
and
in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with 1 to 3 substituents, the substituents being selected independently of one another from the group comprising oxo and C₁-C₄-alkyl,
and
in which phenyl and heteroaryl can be substituted with 1 to 3 substituents, the substituents being selected independently of one another from the group comprising halogen, C₁-C₄-alkyl and C₁-C₄-alkoxy,
and
in which heterocyclyl and heterocyclylcarbonyl can be substituted with 1 to 3 substituents, the substituents being selected independently of one another from the group comprising oxo and C₁-C₄-alkyl,
R⁸ stands for C₁-C₆-alkyl, phenyl, 5- to 7-membered heterocyclyl or 5- or 6-membered heteroaryl,
in which heterocyclyl can be substituted with 1 to 3 substituents, the substituents being selected independently of one another from the group comprising oxo and C₁-C₄-alkyl,
and
in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁹ stands for C₁-C₆-alkyl, C₁-C₄ alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with 1 to 3 substituents, the substituents being selected independently of one another from the group comprising oxo and C₁-C₄-alkyl,
and
in which phenyl and heteroaryl can be substituted with 1 to 3 substituents, the substituents being selected independently of one another from the group comprising halogen, C₁-C₄-alkyl and C₁-C₄-alkoxy,
and
in which heterocyclyl and heterocyclylcarbonyl can be substituted with 1 to 3 substituents, the substituents being selected independently of one another from the group comprising oxo and C₁-C₄-alkyl,
R² stands for hydrogen, chlorine, trifluoromethyl, methyl, ethyl or methoxy,
R³ stands for hydrogen or methyl,
R⁴ stands for hydrogen or methyl,
R⁵ stands for phenyl or 2-thienyl,
in which phenyl and 2-thienyl are substituted with a substituent, the substituent being selected from the group comprising chlorine, fluorine, methyl, ethinyl and methoxy,
or one of their salts, their solvates or the solvates of their salts.

4. The compound according to claim 1, characterized in that
R¹ stands for a compound of formula 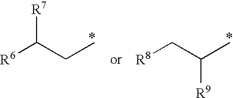
where * is the site of linkage to the indazole,
R⁶ stands for phenyl, 5- to 7-membered heterocyclyl or 5- or 6-membered heteroaryl,
in which heterocyclyl can be substituted with an oxo substituent,
and
in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁷ stands for hydrogen, C₁-C₆-alkyl, C₁-C₄-alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with an oxo substituent,
and
in which phenyl and heteroaryl can be substituted with 1 to 3 substituents, the substituents being selected independently of one another from the group comprising halogen, C₁-C₄-alkyl and C₁-C₄-alkoxy,
and
in which heterocyclyl and heterocyclylcarbonyl can be substituted with an oxo substituent,
R⁸ stands for phenyl, 5- to 7-membered heterocyclyl or 5- or 6-membered heteroaryl,
in which heterocyclyl can be substituted with an oxo substituent,
and
in which phenyl and heteroaryl can be substituted with 1 to 3 substituents, the substituents being selected independently of one another from the group comprising halogen, aminocarbonyl, C₁-C₄-alkyl and C₁-C₄-alkoxy,
R⁹ stands for C₁-C₆-alkyl, C₁-C₄-alkylamino, C₁-C₄-alkoxycarbonyl, 5- to 7-membered heterocyclyl or 5- to 7-membered heterocyclylcarbonyl,
in which alkyl can be substituted with a substituent, the substituent being selected from the group comprising phenyl, 5- to 7-membered heterocyclyl and 5- to 6-membered heteroaryl,
in which heterocyclyl can be substituted with an oxo substituent,
and
in which phenyl and heteroaryl can be substituted with 1 to 3 substituents, the substituents being selected independently of one another from the group comprising halogen, C₁-C₄-alkyl and C₁-C₄-alkoxy,
and
in which heterocyclyl and heterocyclylcarbonyl can be substituted with an oxo substituent,
R² stands for hydrogen or methoxy,
R³ stands for hydrogen,
R⁴ stands for hydrogen,
R⁵ stands for phenyl or 2-thienyl,
in which phenyl and 2-thienyl are substituted with a substituent, the substituent being selected from the group comprising chlorine, fluorine and methyl,
or one of their salts, their solvates or the solvates of their salts.

5. A method of making a compound of formula (I) according to claim 1, characterized in that according to method
[A] a compound of formula 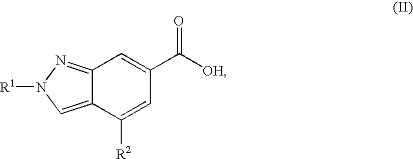
in which
R¹ and R² have the meaning stated in claim 1,
is reacted with dehydrating reagents with a compound of formula 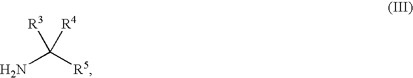
in which
R³, R⁴ and R⁵ have the meaning stated in claim 1,
or
[B] a compound of formula 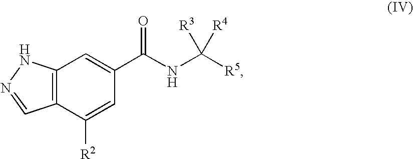
in which
R², R³, R⁴ and R⁵ have the meaning stated in claim 1,
is reacted in the presence of a base with a compound of formula
R¹—X (V),
in which
R¹ the meaning stated in claim 1, and
X stands for halogen, preferably bromine or chlorine,
and then the regioisomers are separated chromatographically,
or
[C] a compound of formula 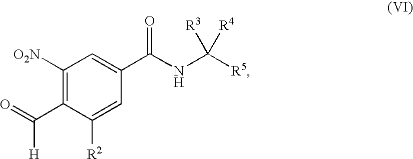
in which
R², R³, R⁴ and R⁵ have the meaning stated in claim 1,
is reacted in a two-stage reaction with a compound of formula
R¹—NH₂ (VII),
in which
R¹ has the meaning stated in claim 1,
first with dehydrating reagents with formation of the imine and then cyclized under reducing conditions.

6. A compound according to claim 1 for the treatment and/or prophylaxis of diseases.

7. (canceled)

8. (canceled)

9. A pharmaceutical composition for the treatment or prophylaxis of a disease comprising a compound according to claim 1 in combination with an inert, nontoxic, pharmaceutically suitable excipient.

10. The pharmaceutical composition of claim 9, wherein the disease is a cardiovascular or thromboembolic disease.

11. A method for treating a cardiovascular disease in a subject by administering a therapeutically effective amount of at least one compound according to claim 1, or a therapeutically effective amount of the pharmaceutical composition of claim 9.

12. A pharmaceutical composition for the treatment or prophylaxis of a disease comprising:
A) at least one compound of formula (I); and
B) at least one other pharmaceutically active substance.

13. The pharmaceutical composition according to claim 12, wherein the at least one other pharmaceutically active substance is a platelet inhibitor, anticoagulant, fibrinolytic, antilipaemic, coronary remedy or vasodilator.

14. The pharmaceutical composition according to claim 12, wherein the at least one other pharmaceutically active substance is rivaroxaban.
